(19) United States
(12) Patent Application Publication
(10) Pub. No.: US 2023/0392124 A1
(43) Pub. Date: Dec. 7, 2023

(54) METHODS AND COMPOSITIONS FOR IMPROVING SC-BETA CELLS OR ENHANCING THEIR UTILITY

(71) Applicants: Jeffrey Millman, St. Louis, MO (US); Kristina Maxwell, St. Louis, MO (US); Nathaniel Hogrebe, St. Louis, MO (US); Leonardo Velazco-Cruz, St. Louis, MO (US); Daniel Veronese Paniagua, St. Louis, MO (US)

(72) Inventors: Jeffrey Millman, St. Louis, MO (US); Kristina Maxwell, St. Louis, MO (US); Nathaniel Hogrebe, St. Louis, MO (US); Leonardo Velazco-Cruz, St. Louis, MO (US); Daniel Veronese Paniagua, St. Louis, MO (US)

(73) Assignee: Washington University, St. Louis, MO (US)

(21) Appl. No.: 17/918,933

(22) PCT Filed: Apr. 16, 2021
(86) PCT No.: PCT/US2021/027786

§ 371 Date: Oct. 14, 2022

Related U.S. Application Data
(60) Provisional application No. 63/010,813, filed on Apr. 16, 2020.

(51) Int. Cl. C12N 5/071; A61K 39/00; A61P 3/10

(52) U.S. Cl. CPC C12N 5/0676; A61K 39/4612; A61K 39/4637; A61P 3/10; C12N 2506/45; C12N 2533/90; C12N 2510/00; C12N 2501/115; C12N 2501/727; C12N 2501/415; C12N 2501/11; C12N 2503/02; C12N 2501/16

(57) ABSTRACT

Among the various aspects of the present disclosure is the provision of methods and compositions for the generation of cells of endodermal lineage and beta cells and uses thereof.

### CROSS-REFERENCE TO RELATED APPLICATIONS

This application is a national stage entry of PCT International Application No. PCT/US2021/027786 filed on 16 Apr. 2021, which claims the benefit of priority to U.S. Provisional Application Ser. No. 63/010,813 filed on 16 Apr. 2020, which are incorporated herein by reference in their entireties.

### STATEMENT REGARDING FEDERALLY SPONSORED RESEARCH OR DEVELOPMENT

This invention was made with government support under DK114233 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “019409-WO-US_Sequence_Listing_ST25.txt” created on 11 Oct. 2022; 20,828 bytes.). The subject matter of the Sequence Listing is incorporated herein by reference in its entirety.

### FIELD

The present disclosure generally relates to methods to enhance SC-beta cell differentiation, maturation, function, and utility of beta cells made from human pluripotent stem cells (SC-beta cells).

### SUMMARY

Among the various aspects of the present disclosure is the provision of methods to enhance SC-beta cell production, functionality, and utility. Applications for the compositions and methods described herein can include diabetes cell replacement therapy; availability of beta cells for study and compound screens. Briefly, therefore, the present disclosure is directed to enhance the availability of beta cells for compound screens and/or diabetes cell replacement therapy. The present teachings include methods for SC-beta cell differentiation and/or maturation that can facilitate large scale up of these processes. An aspect of the present disclosure provides for a method of generating insulin-producing beta cells (e.g., a stem-cell derived beta cell (SC-beta cell)) in a suspension comprising: (Stage 1) providing a stem cell; providing a serum-free media; and/or contacting the stem cell with a TGFβ/Activin agonist or a glycogen synthase kinase 3 (GSK) inhibitor or WNT agonist for an amount of time sufficient to form a definitive endoderm cell; (Stage 2) contacting the definitive endoderm cell with a FGFR2b agonist for an amount of time sufficient to form a primitive gut tube cell; (Stage 3) contacting the primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell; (Stage 4) incubating the early pancreas progenitor cell for at least about 3 days and optionally contacting the early pancreas progenitor cell with a rho kinase inhibitor, a TGF-3/Activin agonist, a smoothened antagonist, an FGFR2b agonist, or a RAR agonist for an amount of time sufficient to form a pancreatic progenitor cell; or (Stage 5) contacting the pancreatic progenitor cell with an AIk5 inhibitor, a gamma secretase inhibitor, SANT1, Erbb1 (EGFR) or Erbb4 agonist, or a RAR agonist for an amount of time sufficient to form an endoderm cell; and/or (Stage 6) allowing the endoderm cell to mature in a second serum-free media for an amount of time sufficient to form a beta cell (e.g., SC-beta cell). In some embodiments, the beta cell is a plurality of beta cells and are re-aggregated into clusters after single cell dispersing and/or seeding into spinner flasks. In some embodiments, the beta cells are single cell dispersed, cryopreserved, and/or thawed, and/or retain function and/or marker expression. In some embodiments, the environments of stage 1-stage 6 cells are modulated via controlling the physical microenvironment of cells through micropatterning, topography (e.g., electrospun fibers, suspension microcarriers), substrate stiffness, modifying the cytoskeleton with soluble small molecules. In some embodiments, the beta cells are planar dispersed on about day 7 and/or replated on microcontact printed patterns (e.g., collagen I). In some embodiments, the stem cells were plated onto micron-sized dots (e.g., 250 μm) or differentiated through stage 1. In some embodiments, the stem cell is a plurality of stem cells or are plated onto electrospun nanofibers or planar differentiated (see e.g., Hogrebe). In some embodiments, the stem cell is a plurality of stem cells and are plated onto soft PDMS plates (about or between about 0.2 kPa or about 2 kPa) or differentiated through stage 1. In some embodiments, the method comprises or further comprises adding cytoskeletal modulating compounds (e.g., s1p) during stage 1, 2, or 3. In some embodiments, hPSCs or SC-beta cells are attached or cultured on bead microcarriers in a bioreactor. In some embodiments, the method comprises or further comprises adding an auxiliary component to ESFM base media (in stage 6), wherein the auxiliary component is capable of modulating GSIS stimulation selected from one or more of: Trace A (e.g., about 1:1000); Trace B (e.g., about 1:1000); Trace C (e.g., about 1:1000); Heparin (e.g., about 10 μg/mL); NEAA (e.g., about 1:100); Vitamin C (e.g., about 250 μM); NaHCO₃ (e.g., about 20 mM); Defined Lipid Mixture (e.g., about 1:1000); Defined Lipid Mixture (e.g., about 1:100); Base; or T3 (e.g., about 1 μM). In some embodiments, an amount of time sufficient to form a primitive gut tube cell (in stage 2) is about 6 days and results in an increased number of PDX1⁺, NKX6.1⁺, or PDX1⁺/NKX6.1+PP2 cells; a decreased number of CHGA⁺ or PDX1⁺/CHGA⁺ PP2 cells; or an increased number of CHGA⁺/NKX6.1⁺ PP2 cells. In some embodiments, an amount of time sufficient to form a primitive gut tube cell (in stage 2) is about 4 days and results in increases CHGA, NKX6.1, or INS gene expression in EN cells. In some embodiments, (optionally in stage 6) the second serum-free media comprises 10% FBS, which results in increased expression of INS, MAF A, SIX2, NKX6-1, SIX3, G6PC2, or MAF B, or decreased GCK expression. In some embodiments, (optionally in stage 6) the second serum-free media comprises Lefty A (optionally, about 1 μg/mL), which results in increased expression of IAPP, SIX2, CHGA, SIX3, G6PC2, or MAF B or decreased NKX6-1 expression. In some embodiments, (optionally in stage 6) the second serum-free media comprises 0.1 μM Alk5i III increased expression of IAPP, MAF A, CHGA, G6PC2, or MAF B or decreased INS or NKX6-1 expression. In some embodiments, the method comprises or further comprises plating a plurality of SC-beta cells. In some embodiments, the method comprises or further comprises plating a plurality of SC-beta cells on a stiff substrate or a soft substrate. In some embodiments, the method comprises or further comprises modulating extracellular matrix (ECM) protein concentration or stiffness to improve SC-beta cells, optionally selected from: plating down SC beta cells; varying matrigel concentration (improved effects on insulin release or genes) on plate down SC beta cells; changing ECM molecules for SC beta cell plate down; varying stiffnesses (increases in stiffness results in gsis performance or SC beta cell maturation); increasing ECM molecules on softer substrate (increasing ECM concentration matures SC beta cells on softer substrate) for SC beta cell plate-down; or different ECM for planar differentiation; or combinations thereof. In some embodiments, the method comprises or further comprises Y (Y27632) or Blebbistatin treatment during stage 4 of differentiation. In some embodiments, the method comprises or further comprises reducing volume of media (e.g., at stage 5, day 1). In some embodiments, the method comprises or further comprises Wnt treatment modification, such as IWP2 treatment during stage 1, days 2-4. In some embodiments, the method comprises or further comprises bFGF treatment during stage 1, results in improved differentiation. In some embodiments, the method comprises or further comprises Betacellulin removal during stage 5. In some embodiments, the method comprises or further comprises IWP2 treatment during stage 2 day 4, resulting in an increase of PDX1 yield at S3. In some embodiments, the second serum-free media in stage 6 does not comprise BC. In some embodiments, the method comprises or further comprises CytoD treatment or high glucose treatment during stage 6, days 1-7 or results in an increase of insulin secretion. Another aspect of the present disclosure provides for a method of evaluating genetic stress of a cell comprising: providing a cell from a subject, wherein if the cell forms a non-pancreatic cell type using the 6 stage differentiation protocol (e.g., Hogrebe, Velezco-cruz, or the aspects or embodiments described herein), or an optimization thereof, the cell is genetically or chemically stressed. Yet another aspect of the present disclosure provides for a method of evaluating chemical stress of a cell comprising: providing an islet cell, exposed to chemical stress, single cell dispersed, or tagged with hashing antibodies to enable single cell RNA sequencing of multiple conditions simultaneously on a single sequencing lane. Yet another aspect of the present disclosure provides for a method of hashing stressed islet cells comprising: providing a human islet cell (optionally treated with one or more of thapsigargin, BFA, cytokine mix, or individual cytokines) or incubated for a time sufficient to form cells sufficient for tagging (e.g., about 48 hours), tagging each condition with a hashing antibody, or detecting the hashing antibodies. Yet another aspect of the present disclosure provides for a method of high-throughput drug screening or measurement of beta cell health comprising: providing a stage 6 INS+/−mcherry SC-islet, single cell dispersing the SC islet, sorting for INS+ SC-β cells, wherein if a reduction of mCherry/INS expression correlates with SC-3 cell health. Yet another aspect of the present disclosure provides for a method of high-throughput drug screening comprising: providing a stage 6 INS+/−mcherry SC-islet, single cell dispersing the SC islet, sorting for INS+ SC-3 cells; and/or optionally treating with a SERCA pump inhibitor (e.g., thapsigargin), which results in a reduction in insulin secretion for high throughput drug screening. Yet another aspect of the present disclosure provides for a method of treating diabetes in a subject comprising transplanting stem cell-derived β cells CRISPR/Cas9-corrected for a diabetes-causing gene variant in WFS1 to restore glucose homeostasis.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. CRISPR/Cas9 correction of WFS1 generates functional WS SC-β cells in vitro. (A) Schematic summary of iPSC generation from patients with WS. (B) Information on the 3 patients, including the genetic location of autosomal recessive pathogenic variants in WFS1 and age of symptom onset. N=none, M=male, F=female, DM=diabetes mellitus, OA=optic atrophy, DI=diabetes insipidus. (C) Gene variants in WFS1 in WS4^{unedit} and WS13^{unedit} iPSCs targeted for CRISPR/Cas9 correction. (D) Schematic of differentiation protocol mimicking embryonic development of pancreatic β cells. (E) Bright field images of stage 6 clusters produced from all cell lines in this study. Scale bar, 500 μm. (F) Static GSIS functional assessment of WS4^{unedit} (n=7), WS9^{unedit} (n=6), and WS13^{unedit} (n=5), WS4^{corr} (n=15) and WS4^{corr-B} (n=4), and WS13^{Corr} (n=4) stage 6 cells in an in vitro static GSIS assay. Data with WS4^{unedit} and WS13^{unedit} cells are regraphed to help with comparisons. Y axes differ between panels. *p<0.05, ****p<0.0001 by one-way paired t-test. †††tp<0.001, ††††p<0.0001 by two-way unpaired t-test comparing to high glucose in unedited cells. iPSC, induced pluripotent stem cell; DE, definitive endoderm; PGT, primitive gut tube; PP, pancreatic progenitor; EP, endocrine progenitor. Act A, activin A; CHIR, CHIR99021; KGF, keratinocyte growth factor; RA, retinoic acid; LDN, LDN193189; T3, triiodothyronine; Alk5i, AIk5 inhibitor type II; ESFM, enriched serum-free medium.

FIG. 2. In vitro characterization of unedited and corrected β cells from iPSCs derived from an individual with WS. (A) Representative flow cytometry dot plots and (B) quantified fraction of cells expressing or co-expressing pancreatic p cell or islet markers for WS4^{unedit} (n=4-7) and WS4^{corr} (n=4-8) stage 6 cells. **p<0.01, ****p<0.0001 by two-way unpaired t-test. (C) Immunostaining of sectioned WS4^{corr} and WS4^{unedit} stage 6 clusters stained for β cell or islet markers. Scale bar, 100 μm. (D) Western blot (left) and quantified intensity (right) of WFS1 protein in stage 0 and stage 6 WS4^{corr} (n=3) and WS4^{unedit} (n=3) cells. **p<0.01, ***p<0.001 by two-way unpaired t-test. (E) Dynamic human insulin secretion of WS4^{corr} (n=5) and WS4^{unedit} (n=4) stage 6 cells and primary human islets (17). Clusters were perfused with 2 mM glucose except where indicated by high glucose (20 mM). CP, C-peptide; GCG, glucagon; SST, somatostatin.

FIG. 3. Transplantation of gene edited patient-derived β cells into mice reverses preexisting diabetes. (A) Schematic of diabetes induction with streptozotocin (STZ), transplantation of stage 6 cells containing WS SC-3 cells, and nephrectomy of the transplanted mice. (B) Blood glucose measurements before and after STZ treatment, and after transplantation with SC-3 cells or human islets. Five groups were studied: Diabetic mice without a transplant (STZ, No Txp; n=7; black), diabetic mice transplanted with WS4^{unedit} stage 6 cells (STZ, WS4^{unedit} Txp; 5×10⁶ cells; n=6; blue), diabetic mice transplanted with human islets (STZ, HI Txp; 4000 IEQ; n=4; dark grey), diabetic mice transplanted with WS4^{corr} stage 6 cells (STZ, WS4^{corr} Txp; 5×10⁶ cells; n=10; red), and non-diabetic mice with a sham transplant (No STZ, No Txp; n=5; light grey). (C) Immunostaining of sectioned kidney explanted from a mouse that had received WS4^{corr} stage 6 cells 2 wk prior. Scale bar, 100 μm. (D) Glucose tolerance test (GTT) 9 d and 10 wk after Txp. Five groups were studied: STZ, No Txp (n=7 at 9 d and 10 wk; black); STZ, WS4^{unedit} Txp (n=6 at 9 d; n=5 at 10 wk; blue); STZ, HI Txp (n=4 at 9 d; dark grey); STZ, WS4^{corr} Txp (n=10 at 9 d; n=9 at 10 wk; red); and No STZ, No Txp (n=5 at 9 d and 10 wk; light grey). (E) Area under the curve (AUC) quantification of GTT data. **p<0.01, ***p<0.001 by Mann-Whitney two-tailed nonparametric test. (F) In vivo GSIS secretion 2 and 10 wk after transplantation for STZ, WS4^{unedit} Txp (n=6 and 5 at 2 and 10 wk; blue); STZ, WS4^{corr} Txp (n=10 and 9 at 2 and 10 wk; red); and STZ, HI Txp (n=4; dark grey) mice at 0 and 60 min after 2 g/kg glucose injection. ns=not significant, **p<0.01 by one-way paired t-test. tp<0.05, tttp<0.001 by two-way unpaired t-test compared to WS4^{unedit} 60 min measurements. (G) Molar ratio of serum human proinsulin to insulin for STZ, WS4^{unedit} Txp (n=5; blue) and STZ, WS4^{corr} Txp (n=9; red) 60 min after 2 g/kg glucose injection 10 wk after transplantation. ***p<0.001 by two-way unpaired t-test. (H) Blood glucose measurements before and after nephrectomy of two STZ, WS4^{corr} Txp (orange) mice compared to remaining non-nephrectomized STZ, WS4^{corr} Txp (n=6; red). CP, C-peptide; GCG, glucagon; SST, somatostatin.

FIG. 4. Single cell transcriptional analysis reveals WS4^{corr} and WS4^{unedit} SC-3 cell populations and off-targets. (A) tSNE projection from unsupervised clustering of transcriptional data from scRNA-seq of WS4^{unedit} and WS4^{corr} stage 6 cells. (B) Calculated percentages of defined cluster populations for WS4^{unedit} and WS4^{corr} stage 6 cells. (C) Heat map of key β cell population gene markers 30 (insulin [INS], chromogranin A [CHGA], SPINK1, ID3) with low/none (grey), medium (yellow), and high (red) expression. NP1, neural progenitor 1; NP2, neural progenitor 2; NP3, neural progenitor 3; PH, polyhormonal; EC, enterochromaffin.

FIG. 5. CRISPR/Cas9 correction of WFS1 improves β cell gene expression in differentiated cells. (A) Violin plots detailing log-normalized gene expression of β cell and islet markers in the WS4^{unedit} (blue) and WS4^{corr} (red) SC-β cell populations defined in FIG. 4. Log fold change and p-values for violin plots are available in TABLE 3A. (B) Real-time PCR analysis of the total stage 6 population measuring expression of β cell and islet genes for WS4^{unedit} (n=6-13; blue) and WS4^{corr} (n=7-14; red). **p<0.01, ***p<0.001, ****p<0.0001 by Man-Whitney two-tailed nonparametric test. (C), Immunostaining of single-cell dispersed WS4^{corr} and WS4^{unedit} stage 6 cells stained for indicated pancreatic and β cell markers. Scale bar, 50 μm.

FIG. 6. CRISPR/Cas9 correction of WFS1 reduces WS SC-β cell stress. (A) Violin plots detailing log-normalized expression of stress genes in the WS4^{unedit} (blue) and WS4^{corr} (red)SC-β cell populations defined in FIG. 4. Log fold-change and adjusted p-values for violin plots available in TABLE 3B. (B) Representative transmission electron microscopy images of ER (top) and mitochondria (bottom) for WS4^{unedit}, WS4^{corr} SC-β cells, and human islets. White dotted lines outline the ER and mitochondria in the cell cytoplasm. Scale bar, 500 nm. (C) Human insulin content (left) and proinsulin/insulin content ratio (right) of WS4^{unedit} (n=7; blue) and WS4^{corr} (n=9; red) stage 6 cells. **p<0.01, ****p<0.0001 by two-way unpaired t-test. (D) Mitochondrial respiration of WS4^{unedit} stage 6 (n=11; blue), WS4^{corr} stage 6 (n=9; red), and human islets (n=6; dark grey) represented as percentage of baseline oxygen consumption rate measurements. Respiration was interrogated by measuring changes in relative OCR after injection with oligomycin (OM), FCCP, and antimycin A (AA)/rotenone (R). (E) Static GSIS functional assessment of stage 6 cells treated with DMSO or thapsigargin (Tg). n=3. *** p<0.001 by two-way unpaired t-test. ns, not significant. (F) Real-time PCR analysis of bulk stage 6 population measuring expression of stress genes after treatment with cytokine mixture (CM), high glucose (Glu), or Tg. n=4. **p<0.01, ***p<0.001 by two-way unpaired t-test compared to ctrl. ctrl=control.

FIG. 7. Additional patient and cell line information. This figure is associated with FIG. 1. (A). Patient number (Pt #) and code previously used to describe 3 WS patients (25) with coordinates of pathogenic variants on WFS1 gene mapped to hg19 and repeated allele information from FIG. 1. (B) Normal 46XX (WS4, WS13) and 46XY (WS9) karyotype of derived iPS cell line (WS4^{unedit}, WS9^{unedit}, WS13^{unedit}) and corrected iPS cell line (WS4^{corr}, WS4^{corr-B} WS13^{corr}). (C) Representative flow cytometry dot plots of dispersed WS4^{unedit} 5 WS9^{unedit}, WS13^{unedit}, WS4^{corr}, WS4^{corr-B}, and WS13^{corr} iPSCs stained for OCT3/4 and NANOG protein. (D) NGS off-target analysis of top 5 off-target sites targeted by WS4 gRNA for CRISPR correction of WFS1 pathogenic variant on Allele 2.

FIG. 8. Additional WS4 stage 6 and human islet analysis. This figure is associated with FIG. 2. (A) Representative flow cytometry dot plots of dispersed WS4^{unedit}, WS4^{corr}, and WS4^{corrB} stage 6 cells for C-peptide, glucagon (GCG), and somatostatin (SST) protein. (B) Representative flow cytometry dot plot of dispersed WS4^{corr-B} stage 6 cells for immunostained C-peptide and NKX6-1, representing percentage of SC-β cells derived from the six-stage differentiation protocol. (C) Quantified fraction of cells expressing or co-expressing β cell or islet markers for WS4^{unedit} (n=4-7) and WS4^{corr-B} (n=4) stage 6 cells. WS4^{unedit} data points are replotted from FIG. 2 to compare with additional WS4^{corr} clone. CP, C-peptide. (D) Immunostaining of sectioned WS4^{corr-B} stage 6 and human islet clusters stained for β cell or islet markers. Scale bar, 100 μm.

FIG. 9. Differentiation progression and efficiency for WS4^{corr} and WS4^{unedit} lines. This figure is associated with FIG. 2. (A) Quantified fraction of cells expressing or co-expressing cell protein markers for WS4^{corr} (n=4) and WS4^{unedit} (n=4) stages 0, 1, 3, 4, and 5 of differentiation. (B) Relative gene expression of key cell markers for WS4^{corr} (n=4) and WS4^{unedit} (n=4) expressed during stages 3, 4, and 5 of differentiation measured with real-time PCR. St, stage; INS, insulin; CHGA, chromogranin A; SST, somatostatin; GCG, glucagon; ISL1, islet1; GCK, glucokinase.

FIG. 10. WFS1 expression during SC-β cell differentiation in WS4^{corr} and WS4^{unedit} lines. This figure is associated with FIG. 2. (A) Relative gene expression of WFS1 for WS4^{corr} (n=4) and WS4^{unedit} (n=4) at various stages of differentiation, normalized to WS4^{corr} stage 0 cells. (B) Immunostaining of single cell dispersed and sectioned clusters of WS4^{corr} and WS4^{unedit} iPSC (stage 0) and stage 6 cells, respectively. Stage 0 stem cells co-stained with stem cell marker, Nanog (green), and stage 6 cells co-stained with SC-β cell marker, c-peptide (CP, green). Scale bar, 50 μm.

FIG. 11. Glucose-stimulated insulin secretion normalized to β cell population. This figure is associated with FIG. 2. (A-B) Static and dynamic GSIS assay of WS4^{unedit} (n=7) and WS4^{corr} (n=15) stage 6 cells normalized to fraction of C-Peptide+/NKX6.1+ cells.

FIG. 12. Additional analysis of WS4^{corr} and WS4^{unedit}SC-β cell transplantations in diabetic mice. This figure is associated with FIG. 3. (A) Serum mouse c-peptide 3 wk after transplantation of WS4^{unedit} (STZ, WS4^{unedit} Txp; n=6, blue) or WS4^{corr} (STZ, WS4^{corr} Txp; n=10; red) stage 6 cells, diabetic mice without a transplant (STZ, No Txp; n=7; white), and nondiabetic mice without a transplant (No STZ, No Txp; n=5; light grey) demonstrating loss of mouse β cell function after STZ injection. n.s. not significant, **** p<0.0001 by one-way ANOVA Tukey multiple comparisons test comparing all groups. (B) Glucose tolerance test (4 g/kg) of mice with STZ treatment to induce diabetes (STZ; n=25; open square) and without STZ treatment (No STZ; n=5; closed circle) before transplantation demonstrating robust induction of pre-existing diabetes. (C) Serum human insulin detected in mice 3 wk after transplantation of WS4^{unedit} (STZ, WS4^{unedit} Txp; n=6; blue) or WS4^{corr} (STZ, WS4^{corr} Txp; n=10; red) stage 6 cells, diabetic mice without a transplant (STZ, No Txp; n=7; white), and nondiabetic mice without a transplant (No STZ, No Txp; n=5; light grey) with random bleeds demonstrating the absence of human insulin in mice without a graft. (D) Body weight of mice 12 wk after transplantation with either WS4^{unedit} (STZ, WS4^{unedit} Txp; n=5; blue) or WS4^{corr} (STZ, WS4^{corr} Txp; n=7; red) stage 6 cells, diabetic mice without a transplant (STZ, No Txp; n=7; white), and nondiabetic mice without a transplant (No STZ, No Txp; n=5; light grey). *p<0.05, n.s., not significant (if not defined) by one-way ANOVA Tukey multiple comparisons test. (E) Body weight of mice after STZ treatment to induce diabetes, before transplantation with WS stage 6 cells. The mice were grouped based on transplantation method as WS4^{unedit} (STZ, WS4^{unedit} Txp; n=6; blue) or WS4^{corr} (STZ, WS4^{corr} Txp; n=10; red) stage 6 cells, diabetic mice without a transplant (STZ, No Txp; n=7; white), and nondiabetic mice without a transplant (No STZ, No Txp; n=5; light grey). n.s., not significant by one-way ANOVA Tukey multiple comparisons test. (F) Weekly body weight measurement before STZ treatment, after STZ treatment to induce diabetes, and after TXP of WS4^{unedit} (STZ, WS4^{unedit} Txp; n=4-6) and WS4^{Corr} (STZ, WS4^{corr} Txp; n=6-10) stage 6 cells in diabetic mice, diabetic mice without a transplant (STZ, No Txp; n=5-8), and nondiabetic (ND) mice without a transplant (No STZ, No Txp; n=5). (G) Immunostaining of sectioned kidneys explanted from STZ, WS4^{corr} TXP mice 2 weeks after transplantation immunostained for C-peptide (CP), somatostatin (SST), glucagon (GCG), or nuclear marker DAPI. White dashed line borders the graft in the mouse kidney capsule. This is a lower magnification image of what is shown in FIG. 2. Blood cells are noted to have autofluoresce. Scale bar=100 μm. (H) Images of explanted kidneys from TXP of WS4^{unedit} (STZ, WS4^{unedit} Txp) and WS4^{corr} (STZ, WS4^{corr} Txp) stage 6 cells into diabetic mice. We sacrificed one WS4^{unedit} and one WS4^{corr} mouse at 2 wk, and one WS4^{unedit and} two WS4^{corr} SC-β cell mice at 10-12 wk, and the rest at the end of the experiment.

FIG. 13. Additional analysis of WS4^{corr} and WS4^{unedit}SC-β cell scRNA-seq. This figure is associated with FIG. 4. (A) Mitochondrial count per cell distribution represented as violin plot for both WS4^{corr} and WS4^{unedit} stage 6 cells. Cells above the red line threshold were filtered out for analysis. (B) Gene count per cell distribution represented as violin plot for both WS4^{corr} and WS4^{unedit} stage 6 cells. Cells above the red line threshold are considered apoptotic and were filtered out for analysis. (C) Gene expression of defined population-specific markers presented as low (grey), medium (yellow), and high (red) values in the cell population clusters for WS4^{unedit} and WS4^{corr} stage 6 cells. (D) tSNE projection from unsupervised clustering of WS4^{unedit} and WS4^{corr} stage 6 cells combined with canonical correlation analysis. Clusters defined based on genes differentially expressed between clustered population.

FIG. 14. Additional analysis of WS4^{corr} and WS4^{unedit}SC-β cell scRNA-seq for off-targets. This figure is associated with FIG. 4. (A) Violin plots detailing log-normalized gene marker expression of genes defining off-target populations comparing all sequenced cells from WS4^{unedit} (blue) and WS4^{corr} (red) stage 6 cells. (B) (top) Real-time PCR measuring population level gene expression of off-target genes WS4^{unedit} (n=3-4; blue) and WS4^{corr} (n=3-4; red) stage 6 cells. (bottom) Real-time PCR measuring population level gene expression of off-target genes in WS4^{unedit} (n=4; blue) and WS4^{corr} (n=4; red) in definitive endoderm cells (stage 1). *p<0.05, **p<0.01, ****p<0.0001 by two-way unpaired t-test.

FIG. 15. Additional analysis of differences in SC-β cell beta and islet markers for WS4^{corr} clones and WS4^{unedit} lines. This figure is associated with FIG. 5. (A) Real-time PCR analysis of the total stage 6 population measuring expression of β cell and islet genes for WS4^{unedit} (n=7-13; blue) and WS4^{corr-B} (n=4; striped red). WS4^{unedit} values are replotted from FIG. 5B. *p<0.05, **p<0.01, ***p<0.001 by Mann-Whitney two-tailed nonparametric test. (B) Immunostaining of single-cell dispersed WS4^{corr-B} stained for indicated pancreatic and R cell markers. Scale bar, 50 μm.

FIG. 16. Additional analysis of ER stress gene expression. This figure is associated with FIG. 6. (A) Real-time PCR analysis of the total stage 6 population measuring expression of stress markers. n=3-13.

FIG. 17. Additional analysis of TEM and mitochondrial respiration. This figure is associated with FIG. 6. (A) Representative TEM images for WS4^{corr} and WS4^{unedit} stage 6 cells and human islets without dashed line marking key organelles to improve visibility. Scale bar, 500 nm. (B) Human insulin content (left) and proinsulin/insulin content ratio (right) of WS4^{corr-B} (n=4). (C) Mitochondrial respiration of WS4^{corr} (n=6) and WS4^{unedit} (n=5) iPSC represented as percentage of baseline oxygen consumption rate measurements using the Seahorse XFe24.

FIG. 18. Treatment of SC-β cells with chemical stressors. This figure is associated with FIG. 6. (A) Real-time PCR analysis of bulk stage 6 population treated with cytokine mixture (CM), high glucose (Glu), or thapsigargin (Tg) measuring stress markers. n=3. (B) Western blot analysis of bulk stage 6 population treated with Tg measuring stress or insulin processing protein markers. Quantitation is normalized by α-tubulin. n=3. PDI, protein disulfide isomerase.

FIG. 19. Stress marker measurements of WS4^{corr-B} and human islets. This figure is associated with FIG. 6. Real-time PCR analysis of cells treated with high glucose (Glu) or thapsigargin (Tg) measuring stress markers.

FIG. 20. SC-β cells are capable of re-aggregating into clusters within spinner flasks after dispersion from clusters. Cells seeded: 5 million. Cells retrieved after re-aggregation: 4.1 M.

FIG. 21. SC-β cells re-aggregate in spinner flasks without loss of function. Error bars=SD; n=4.

FIG. 22. Thawed cryopreserved SC-β cells re-aggregate after thawing when cultured in suspension. 83% retrieval after re-aggregation relative to cryopreserved cells.

FIG. 23. Thawed cryopreserved SC-β cells adhere when cultured on Matrigel® coated plastic. 93% retrieval after plate down relative to cryopreserved cells.

FIG. 24. Thawed cryopreserved SC-β cells maintain marker expression.

FIG. 25. Thawed cryopreserved SC-β cells remain function showing glucose stimulated insulin secretion.

FIG. 26. S6d14: planar stage 6 cells dispersed on s6d7 and replated on microcontact printed patterns (collagen I).

FIG. 27. S6d14, 250 μm quantum dots. Planar stage 6 cells dispersed on s6d7 and replated on microcontact printed patterns (collagen I).

FIG. 28. S6d14, 250 μm quantum dots. Planar stage 6 cells dispersed on s6d7 and replated on microcontact printed patterns (collagen I).

FIG. 29. S6d14. Planar stage 6 cells dispersed on s6d7 and replated on microcontact printed patterns (collagen I).

FIG. 30. In stage 6, cell shape and cytoskeletal arrangement don't necessarily increase traditional maturation genes but instead are important for the proper insulin secretion machinery.

FIG. 31. S1d1; Microcontact printing 250 μm dots.

FIG. 32. S1d2; Microcontact printing 250 μm dots

FIG. 33. S2d1; Microcontact printing 250 μm dots.

FIG. 34. Patterning stem cells can strongly influence expression of genes associated with various germ layers.

FIG. 35. s1d1; Stem cells were plated onto electrospun nanofibers and differentiated with the planar SC-β cell protocol.

FIG. 36. S2d1; Stem cells were plated onto electrospun nanofibers and differentiated with the planar SC-β cell protocol.

FIG. 37. S2d1; Changing substrate topography experienced by stem cells with electrospun fibers can strongly influence expression of genes associated with various germ layers.

FIG. 38. S5d1; Later in the protocol, the fibers also influence genes associated with beta cells as well as other endodermal lineages.

FIG. 39. S6d1; Later in the protocol, the fibers also influence genes associated with beta cells as well as other endocrine cell types.

FIG. 40. S1d4; Stem cells were plated onto soft PDMS plates (0.2 and 2 kPa) and differentiated through stage 1. Changing substrate stiffness experienced by stem cells can strongly influence expression of genes associated with various germ layers.

FIG. 41. S1d1; Small molecules that influence the state of the cytoskeleton were added for first 24 hours of various stages of the SC-β cell protocol.

FIG. 42. S2d1; Small molecules that influence the state of the cytoskeleton were added for first 24 hours of various stages of the SC-β cell protocol.

FIG. 43. S2d1; Adding these cytoskeletal modulating compounds during stage 1 can strongly influence expression of genes associated with various germ layers.

FIG. 44. S2d1; In particular, s1p (which induces actin polymerization) greatly increases the expression of the mesoderm marker Brachyury T.

FIG. 45. S5d1; Adding these cytoskeletal modulating compounds during stage 2 or 3 can strongly influence expression of genes associated with pancreatic progenitors as well as other endodermal lineages. *compounds added during first 24 hours of either s2 or s3.

FIG. 46. S6d32; Stage 6 cells were single cell dispersed clusters at s6d20 and seeded onto matrigel-coated suspension beads.

FIG. 47. Stage 6 cells are able to attach to the surface of microcarriers.

FIG. 48. S6d32; Attaching stage 6 cells to microcarriers may influence SC-β cell gene expression and GSIS.

FIG. 49. Undifferentiated stem cells can also be successfully attached and cultured on bead microcarriers in a bioreactor.

FIG. 50. SC-β cells respond to chemical stress. (A) Increased ER stress gene expression; (B) Increased ER stress proteins; (C) Reduced glucose stimulated insulin secretion. ns not specific, *p<0.05, **p<0.01, ***p<0.001, ****p<0.0001

FIG. 51. SC-β cells with diabetes-causing mutations respond to genetic stress in vitro. (A) Reduced glucose stimulated insulin secretion; (B) Reduced Insulin Content; (C) Increased Proinsulin/Insulin Ratio. Scale bar=500 nm, **p<0.01, ++++p<0.0001, **** p<0.0001.

FIG. 52. SC-β cells with diabetes-causing mutations respond to genetic stress in vitro. (A) Reduced maximal respiratory capacity. (B) Swollen ER and fragmented mitochondria.

FIG. 53. SC-β cells with diabetes-causing mutations respond to genetic stress in vivo. Thus figure shows unable to regulate glucose; reduced glucose stimulated insulin secretion; and increased proinsulin/insulin ratio. Scale bar=50 μm, ns not significant, +p<0.05, **p<0.01, *** and +++p<0.001.

FIG. 54. Genetically stressed SC-islets produce non-pancreatic cell types from 6 stage differentiation protocol. Reduction in SC-β yields from SC-islet differentiation. Scale bar=50 μm, *p<0.05, **p<0.01, ****p<0.0001.

FIG. 55. Genetically stressed SC-islets produce non-pancreatic cell types from 6 stage differentiation protocol. Many non-pancreatic cell types are identified.

FIG. 56. Cadaveric Human Islets with Stress. Increased ER stress gene expression. Islets survive for further transcriptome analysis. Scale bar=100 μm.

FIG. 57. Hashing of Stressed Cadaveric Human Islets.

FIG. 58. mCherry fluorescence increases at late stages of SC-islet differentiation. Scale bar=100 μm.

FIG. 59. mCherry fluorescence increases at late stages of SC-islet differentiation.

FIG. 60. INS-mCherry+ cells with thapsigargin treatment reduces mCherry fluorescence.

FIG. 61. De-differentiation signatures occur in INS-mCherry+ cells with thapsigargin treatment.

FIG. 62. NAHCO₃ in ESFM reduces stimulation index by increasing insulin secretion at low glucose. Adding individual components on top of base media increases stimulation GSIS.

FIG. 63. Defined Lipid Mixture at 1:100 reduced stimulation index by increasing insulin secretion at low glucose.

FIG. 64. Scheme modified from Velazco-cruz 2019 (see FIG. 64).

FIG. 65. 6 days of Stage 2 increases the number of PDX1+, NKX6.1+, and PDX1+/NKX6.1+PP2's.

FIG. 66. 6 days of Stage 2 slightly decreases the number of CHGA+ and PDX1+/CHGA+PP2's.

FIG. 67. 6 days of Stage 2 slightly increases the number of CHGA+/NKX6.1+PP2's.

FIG. 68. Figure from Hogrebe et al. (2020) FIG. 69. 4 days of Stage 2 is optimal for CHGA but not NKX6.1 and PDX1 gene expression in PP2 cells.

FIG. 70. 4 days of Stage 2 increases CHGA, NKX6.1, and INS gene expression in EN cells.

FIG. 71. Relative expression of INS.

FIG. 72. Relative expression of IAPP.

FIG. 73. Relative expression of MAF A.

FIG. 74. Relative expression of SIX2.

FIG. 75. Relative expression of GCK.

FIG. 76. Relative expression of CHGA.

FIG. 77. Relative expression of NKX6-1.

FIG. 78. Relative expression of SIX3.

FIG. 79. Relative expression of G6PC2.

FIG. 80. Relative expression of MAF B.

FIG. 81. Relative expression of INS, IAPP, MAF A, SIX2, GCK, CHGA, NKX6-1, SIX3, G6PC2, and MAF B.

FIG. 82. Relative expression of INS, IAPP, MAF A, SIX2, GCK, CHGA, NKX6-1, SIX3, G6PC2, and MAF B.

FIG. 83. Improving GSIS by plating down SC beta cells.

FIG. 84. Plating Down with Different Matrigel coating condition.

FIG. 85. Improvements do not depend on adhesion molecules.

FIG. 86. Stiffness shows trends in GSIS improvement.

FIG. 87. Increasing ECM concentration matures SC beta cells on a softer substrate (25 kPa).

FIG. 88. Different ECMs used for planar differentiation; Flow cytometry s6d7—pdx1 nkx61.

FIG. 89. Different ECMs used for planar differentiation; Flow cytometry s6d7—pdx1 cpeptide.

FIG. 90. Y and Blebbistatin treatment during S4 improves PDX1/NKX61 co-expression.

FIG. 91. Reduced volume maintains good SC beta cell differentiation.

FIG. 92. IWP2 during Sd2-d4 promotes PDX1 yield at S3.

FIG. 93. bFGF treatment during Stage 1 improves differentiation.

FIG. 94. BC not required for beta cell induction.

FIG. 95. CytoD or high glucose treatment during s6d1-7 increases insulin secretion.

### DETAILED DESCRIPTION

The present disclosure is based, at least in part, on the discovery that the following modifications to the protocol and for making and studying stem cell-derived beta cells (SC-β cells) enhanced the directed differentiation:
Re-aggregating stage 6 cells in spinner flasks
Cryopreservation of SC-Beta cells
Microenvironmental cues to enhance SC-β cell differentiation and maturation
How SC-β cells respond to chemical and genetic stress
Cell hashing stressed β cells
Using mCherry/INS reporter cell line to study β cell health
Refining stage 6 enriched serum-free media (ESFM) for SC-β cells
Stage 2 duration affects pancreatic differentiation
Compounds to improve SC-beta cells
ECM proteins and stiffness influence SC-beta cell function and maturation
Other modifications to the protocol

A 6-step differentiation protocol was modified from Pagliuca et al. Cell 2014, by multiple approaches for enhancing differentiation, maturation, and function of beta cells made from human pluripotent stem cells (SC-beta cells). In addition, we describe other new technologies that help with the utility of SC-beta cells.

One aspect of the present disclosure provides methods to enhance differentiation, maturation, and function of beta cells made from human pluripotent stem cells. These methods facilitate large scale up of SC-beta cell production. They ensure greater quality control and assurance of SC-beta cell product from large batches. These cells could improve diabetes cell replacement therapy and increase the availability of beta cells for study and compound screens.

Methods, unless otherwise stated, can be as described in U.S. application Ser. No. 15/788,989 (WU Ref No. 016681-ORD1/1); PCT/US2019/032643 (WU Ref No. 018619/WO); or 62/937,825 (WU Ref No. 019270/US), incorporated herein by reference in their entireties.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell. “Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, WFS1 signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate correction to a diabetes-causing pathogenic variant in Wolfram syndrome 1 (WFS1) in iPSCs derived from a patient with Wolfram syndrome (WS) by genome editing can result in protection from diabetes.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for diabetes to target cells by the correction, removal, or addition of signals such as WFS1 (e.g., activate (e.g., CRISPRa), upregulate, downregulate genes).

For example, the methods as described herein can comprise a method for altering a target polynucleotide sequence in a cell comprising contacting the polynucleotide sequence with a clustered regularly interspaced short palindromic repeats-associated (Cas) protein.

Gene Therapy and Genome Editing

Gene therapies can include inserting a functional gene with a viral vector. Gene therapies for diabetes are rapidly advancing.

There has recently been an improved landscape for gene therapies. For example, in the first quarter of 2019, there were 372 ongoing gene therapy clinical trials (Alliance for Regenerative Medicine, 5/9/19).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | |
|---|---|
| | Associated experimental |
| Strategy | models |

Viral Vectors

| | | |
|---|---|---|
| Retroviruses | Retroviruses are RNA viruses | Murine model of MPS VII |
| | transcribing their single-stranded | Canine model of MPS VII |
| | genome into a double-stranded | |
| | DNA copy, which can integrate | |
| | into host chromosome | |
| Adenoviruses (Ad) | Ad can transfect a variety of | Murine model of Pompe, Fabry, |
| | quiescent and proliferating | Walman diseases, |
| | cell types from various species | aspartylglucosaminuria |
| | and can mediate | and MPS VII |
| | robust gene expression | |
| Adeno-associated | Recombinant AAV vectors | Murine models of Pompe, Fabry |
| Viruses (AAV) | contain no viral DNA and can | diseases, |
| | carry ~4.7 kb of foreign | Aspartylglucosaminuria, Krabbe |
| | transgenic material. They | disease, Metachromatic |
| | are replication defective and can | leukodystrophy, MPS I, MPSII, |
| | replicate only while | MPSIIIA, MPSIIIB, MPSIV, |
| | coinfecting with a helper virus | MPSVI, MPS VII, CLN1, CLN2, |
| | | CLN3, CLN5, CLN6 |

Non-viral vectors

| | | |
|---|---|---|
| plasmid DNA | pDNA has many desired | Mouse model of Fabry disease |
| (pDNA) | characteristics as a gene | |
| | therapy vector; there are no | |
| | limits on the size or genetic | |
| | constitution of DNA, it is | |
| | relatively inexpensive to supply, | |
| | and unlike viruses, antibodies | |
| | are not generated | |
| | against DNA in normal | |
| | individuals | |
| RNAi | RNAi is a powerful tool for gene | Transgenic mouse strain |
| | specific silencing that | Mouse models of acute liver |
| | could be useful as an enzyme | failure |
| | reduction therapy or | Mice with hepatitis B virus |
| | means to promote read-through | Fabry mouse |
| | of a premature stop | |
| | codon | |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

Screening

Also provided are methods for screening. The screening method can comprise providing a generated cell by any of the methods described herein and introducing a compound or composition (e.g., a secretagogue) to the cell. For example, the screening method can be used for drug screening or toxicity screening on any cell of endodermal lineage or beta cell provided herein.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Formulation

The agents and compositions described herein can be formulated in any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of using generated cells for cell replacement therapies or stem cell transplant. For example, the disclosed compositions and methods can be used to treat diabetes or other disease associated with dysfunctional endodermal cells in a subject in need administration of a therapeutically effective amount of cells of endodermal lineage or beta cells, so as to induce insulin secretion.

Methods described herein are generally performed on a subject in need thereof. A subject in need of the therapeutic methods described herein can be a subject having, diagnosed with, suspected of having, or at risk for developing diabetes or other disease associated with dysfunctional endodermal cells. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of cells of endodermal lineage (e.g., hepatocytes, insulin-expressing cells (e.g., β cells, SC-β cells), intestinal cells) is, for example, that amount that would cause the desired therapeutic effect in a subject while minimizing undesired side effects.

In various embodiments, an effective amount of endodermal lineage or beta cells described herein can respond to glucose by secretion of insulin. In various embodiments, an effective amount of cells described herein can treat diabetes or other disease associated with dysfunctional endodermal cells, substantially inhibit diabetes or other disease associated with dysfunctional endodermal cells, slow the progress of diabetes or other disease associated with dysfunctional endodermal cells, or limit the development of diabetes or other disease associated with dysfunctional endodermal cells.

According to the methods described herein, administration can be a cell transplantation, cell implantation, parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of beta cells or cells of endodermal lineage can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to induce insulin secretion.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of cells of endodermal lineage or beta cells can occur as a single event or over a time course of treatment. For example, cells of endodermal lineage or beta cells can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for diabetes or other disease associated with dysfunctional endodermal cells.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery, tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting or grafting stem cells can be used to regenerate diseased tissues, such as transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogenic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig-derived cells can be transplanted into humans. Xenogeneic cell therapies can involve human cell transplantation into experimental animal models for assessment of efficacy and safety or enable xenogeneic strategies to humans as well.

KITs

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to stem cells, media, and factors as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Gene-Edited Human Stem Cell-Derived B Cells from a Patient with Monogenic Diabetes Reverse Preexisting Diabetes in Mice

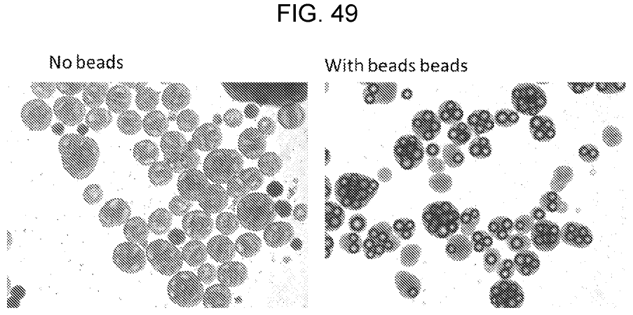

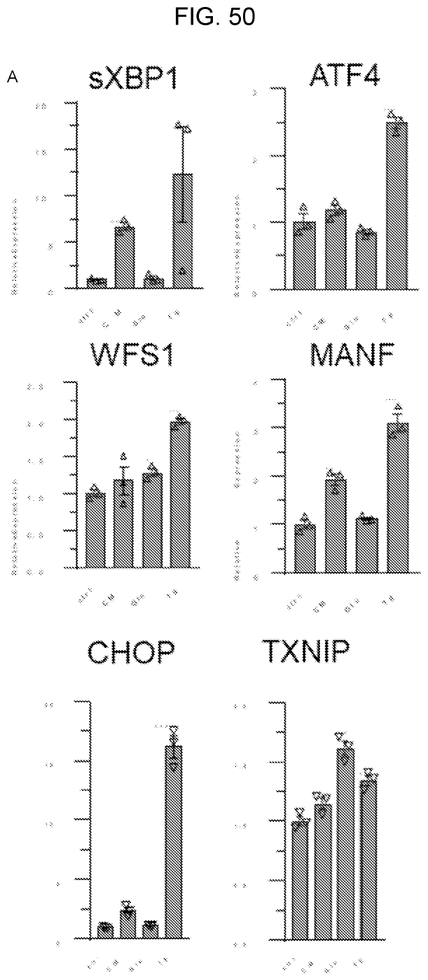

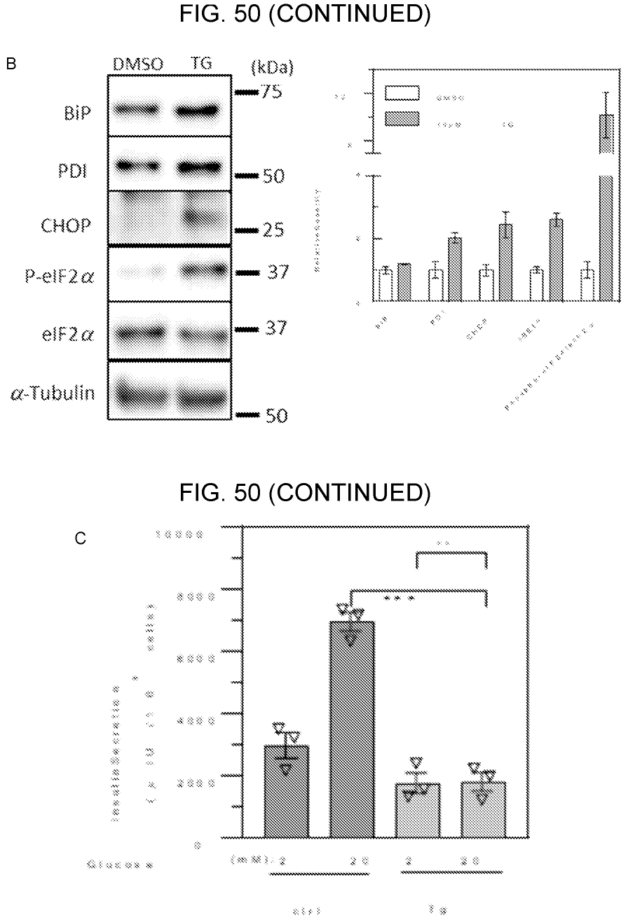

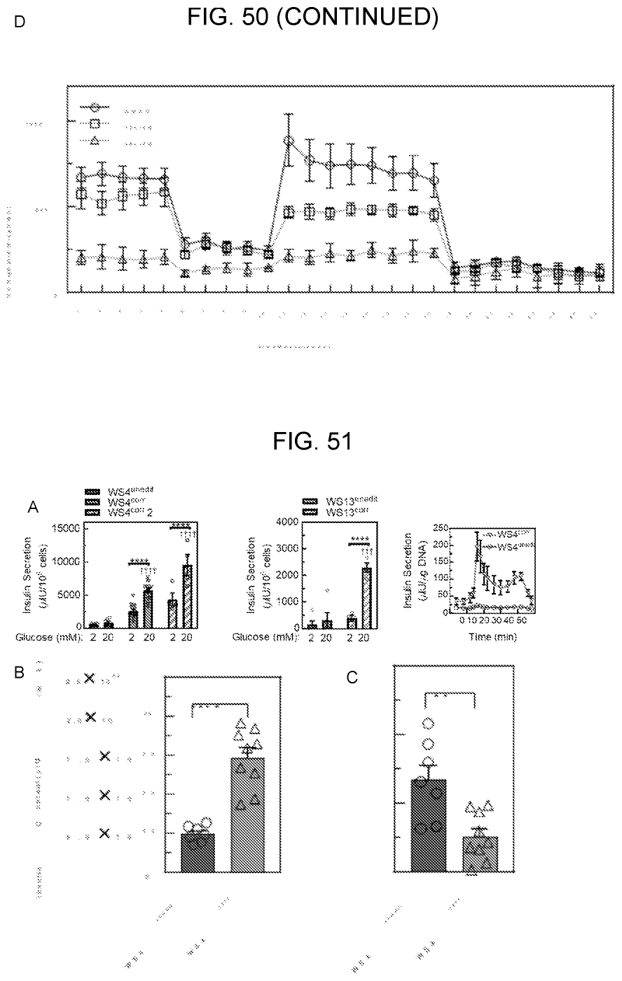

The following example describes patient stem cell-derived β cells CRISPR/Cas9-corrected for a diabetes-causing gene variant in WFS1 restore glucose homeostasis when transplanted into diabetic mice. See also FIG. 50-FIG. 55, Section (IV) in Example 2.

Abstract

Differentiation of insulin-producing pancreatic β cells from induced pluripotent stem cells (iPSCs) derived from patients with diabetes promises to provide autologous cells for diabetes cell replacement therapy. However, current approaches produce patient iPSC-derived β (SC-β) cells with poor function in vitro and in vivo. Here, we used CRISPR-Cas9 to correct a diabetes-causing pathogenic variant in Wolfram syndrome 1 (WFS1) in iPSCs derived from a patient with Wolfram syndrome (WS). After differentiation to β cells with our recent six-stage differentiation strategy, corrected WS SC-β cells performed robust dynamic insulin secretion in vitro in response to glucose and reversed preexisting streptozocin-induced diabetes after transplantation into mice. Single-cell transcriptomics showed that corrected SC-β cells displayed increased insulin and decreased expression of genes associated with endoplasmic reticulum stress. CRISPR-Cas9 correction of a diabetes-inducing gene variant thus allows for robust differentiation of autologous SC-β cells that can reverse severe diabetes in an animal model.

Introduction

Derivation of induced pluripotent stem cells (iPSCs) from patients followed by differentiation into disease-relevant cell types holds great promise for in vitro disease modeling, drug screening, and autologous cell replacement therapy for multiple diseases (1, 2). Diabetes mellitus is caused by the death or dysfunction of insulin-producing β cells within the pancreas. Although insulin injections are often used to replace this lost function (3), long-term complications can arise (4). Alternatively, transplantation of cadaveric allogeneic islets containing β cells has been performed successfully, demonstrating the feasibility of a cell therapy approach that is however limited due to low donor numbers and the need for immunosuppressant drugs (5-7).

Stem-cell derived β cells (SC-β cells) differentiated from iPSCs derived from patients with diabetes would provide a source of autologous replacement cells (8), but the lack of robust physiological function of these cells has been an unmet need in the field (9). Specifically, prior reports using patient iPSCs have generated pancreatic or endocrine progenitors lacking β cell identity (10-14). Recently we and others have developed differentiation strategies with human embryonic stem cells (hESCs) to generate functional non-progenitor SC-β cells in vitro as an alternative source of replacement cells (15-17). Although these and similar approaches have been used in vitro to generate iPSC- or nuclear transfer stem cell-derived β cells from patients with Type 1 (18, 19), Type 2 (20), and neonatal diabetes (21, 22), these cells have shown only modest function in vitro and in vivo. In particular, unlike with primary β cells, these SC-β cells derived from patients with diabetes required long times after transplantation (12-19 wk) to functionally mature and normalize blood glucose in modestly diabetic mice or had a high failure rate, being unable to achieve normoglycemia or having formation of overgrowths. In addition, they were not transplanted into mice with pre-existing diabetes and in vitro dynamic glucose-stimulated insulin secretion (GSIS) was not tested. To overcome these limitations, we recently developed a differentiation protocol that leverages a previously unknown role of the cytoskeleton in pancreatic fate choice to produce highly functional SC-β cells across multiple cell lines (23).

Single pathogenic gene variants that cause diabetes can be corrected in iPSCs (21, 22) using CRISPR/Cas9 gene editing (24). One applicable condition is Wolfram Syndrome (WS), a rare autosomal recessive disorder caused by pathogenic variants in the WFS1 gene (25, 26), which cause chronic endoplasmic reticulum (ER) stress that stimulates the unfolded protein response and eventually causes the death of β cells (27-29). Individuals with WS develop diabetes in childhood along with other ailments, including optic nerve atrophy and neurodegeneration (26). ER stress is a common feature shared with all forms of diabetes and other diseases (30-35). There is currently no effective treatment for WS or ER stress-related diseases (26).

Here we combined our recently devised differentiation strategy (23) with CRISPR/Cas9 gene editing of WFS1 in patient-derived iPSCs to generate autologous, gene-corrected SC-β cells from patients with WS. We assessed the differentiation efficacy and glucose-stimulated insulin secretion of the gene-corrected, patient-derived SC-β cells as compared to unedited cells both in vitro and in vivo when transplanted into a mouse model of diabetes. We also used single-cell RNA sequencing (scRNA-seq) to further characterize these cells and followed up these observations with assessment of ER and mitochondrial stress. Our work highlights that CRISPR/Cas9 correction of patient-derived iPSCs can be used to generate cells with utility in diabetes cell replacement therapy.

Results

Gene-Corrected WS SC-β Cells Display Dynamic Glucose Stimulated Insulin Secretion and Express β Cell Markers

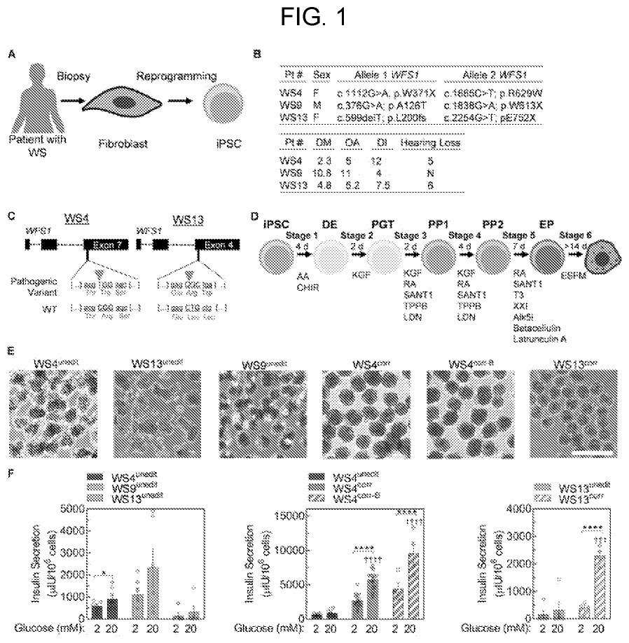

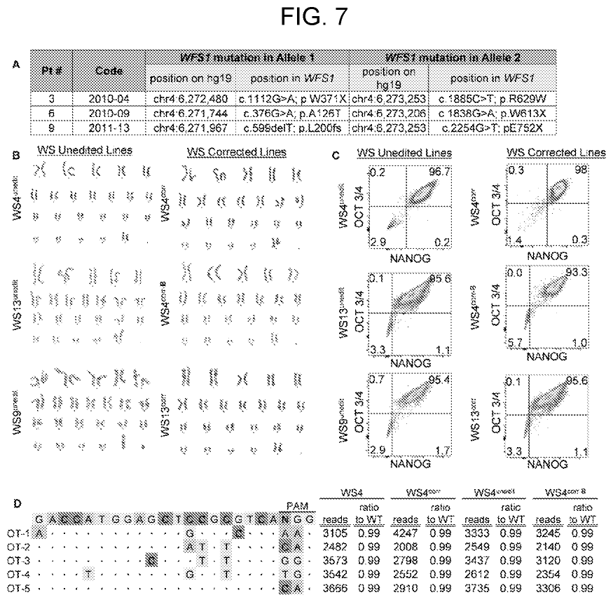

We started our study with iPSC lines reprogrammed from fibroblasts of two patients with WS, previously (36) (WU.WOLF-09 and WU.WOLF-13, designated WS9^{unedit} and WS13^{unedit}, respectively, in this study) and also generated iPSCs from skin fibroblasts of one additional, previously described patient with Wolfram Syndrome (25) (WU.WOLF-04, designated WS4^{unedit} in this study). We used CRISPR/Cas9 to correct the pathogenic variants in the WFS1 gene for patients WU.WOLF-04 and WU.WOLF-13, generating multiple corrected iPSC clones designated WS4^{corr}, WS4^{corr-B}, and WS13^{corr} (FIG. 1A-FIG. 1C, FIG. 7, TABLE 1). We attempted to adapt the unedited iPSC lines from all three patients to suspension culture for propagation and differentiation to generate SC-β cells as previously reported (17). However, we were unable to establish these suspension cultures due to extensive cell death and low cell yields. Due to these challenges, we did not continue to attempt suspension differentiations for corrected clones and instead used our recent differentiation protocol based on cytoskeletal modulation (23). We differentiated WS iPSCs into SC-β cells with small molecules and growth factors to recapitulate pancreatic development using attachment culture and aggregated the final cell population into islet-like clusters for assessment (FIG. 1D-FIG. 1E). Differentiation of unedited iPSC lines from all three patients produced cells capable of secreting insulin (FIG. 1F). However, our three CRISPR/Cas9-corrected cells lines (WS4^{corr}, WS4^{corr-B}, and WS13^{corr}) were more glucose responsive, secreting 6.4±1.4×, 10.6±1.3×, and 7.2±0.6× more insulin at 20 mM glucose compared to their unedited isogenic and patient-matched controls, respectively (FIG. 1F). These data indicate that Wolfram Syndrome-causing pathogenic variants in WFS1 contribute to defects in GSIS that can be corrected with gene editing.

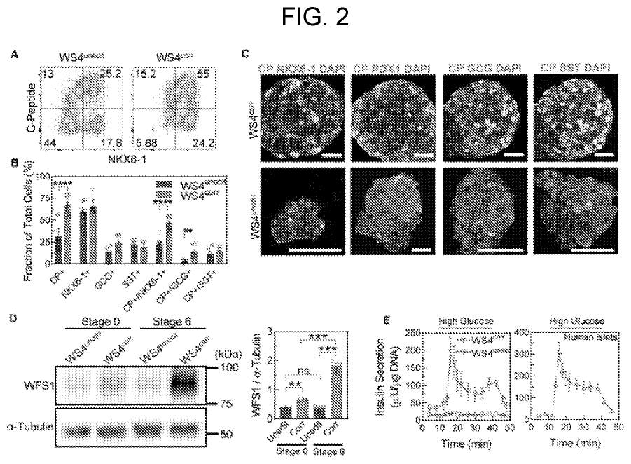

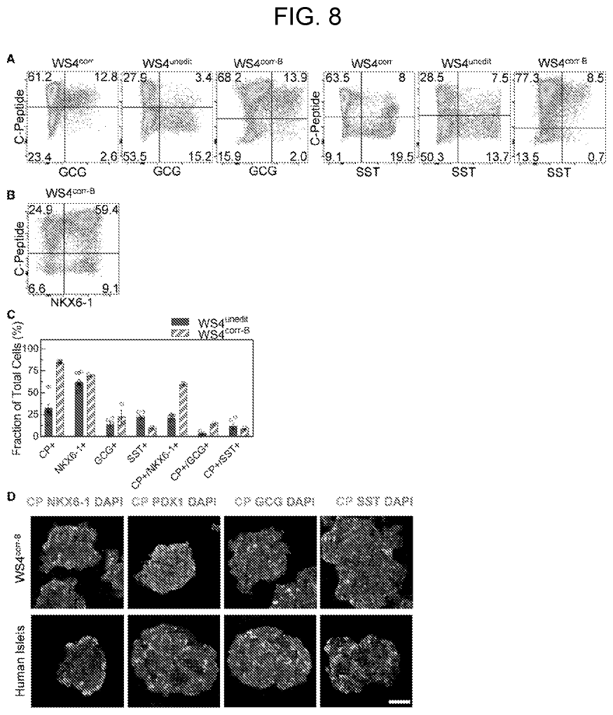

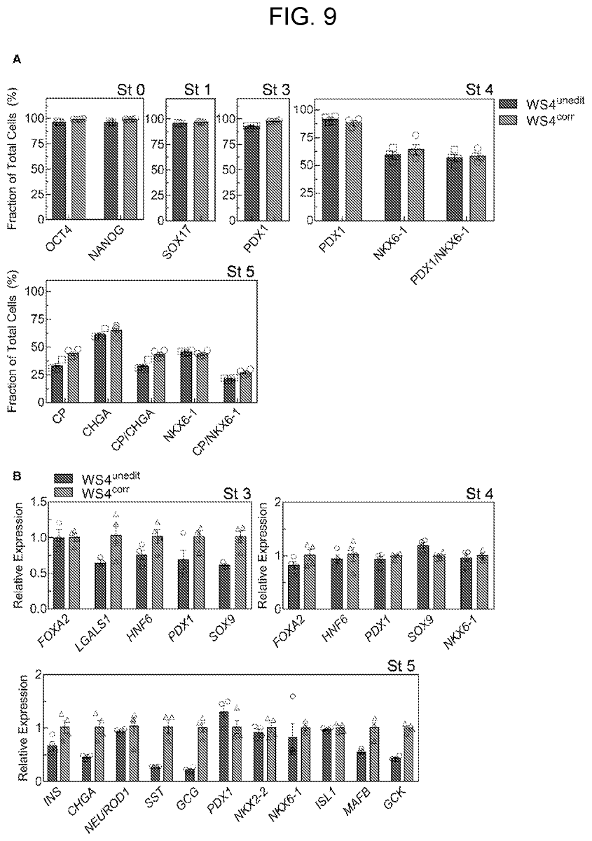

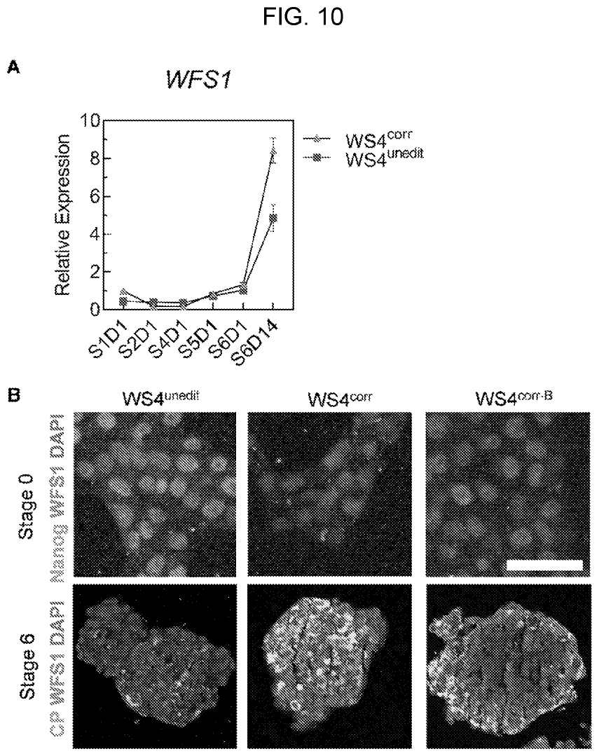

We performed further characterization of the islet-like clusters from patient WS4 expressing a, P, and b cell hormones with and without CRISPR/Cas9 correction (FIG. 8A). WS4^{unedit}, WS4^{corr}, and WS4^{corr-B} all produced C-peptide+ cells that co-stained with β cell transcription factors NKX6-1 and PDX1 (FIG. 8A-FIG. 8C, FIG. 8B-FIG. 8D). However, SC-β cell generation, as indicated by C-peptide+/NKX6-1+ cells, was lower for WS4^{unedit} (22±2%) cells compared to WS4^{corr} (47±2%) and WS4^{corr-B} (59.1±1%) cells (FIG. 2A-FIG. 2C, FIG. 8B-FIG. 8D). The fractional yield of SC-β cells from corrected clones was similar to our previously reported SC-β cells derived from donors without diabetes (non-diabetic SC-β cells) (17, 23). The early stages of differentiation generated cells with similar relative gene expression and fraction of cells expressing key progenitor markers (FIG. 9). We began to detect differences in differentiation efficiency at stage 5, when endocrine cell induction predominantly occurs (15), and these differences amplified during stage 6 (FIG. 2C). The mechanistic cause of WS4^{unedit} SC-β cell differentiation defects is unknown, but other reports have shown organelle calcium and oxidative stress influence the differentiation of other cell types (37, 38). WFS1 gene and protein expression were detectable across all stages of differentiation. However, the detrimental effects of WFS1 pathogenic variants on differentiation towards SC-β cells appeared mostly during later stages of differentiation when WFS1 expression increased (FIG. 2D, FIG. 10). We observed lower expression of WFS1 protein in WS4^{unedit} compared to WS4^{corr} cells (FIG. 2D). In prior reports, variant WFS1 protein and RNA were shown to be unstable and undergo proteasomal degradation, cellular depletion, and non-sense-mediated decay (39, 40).

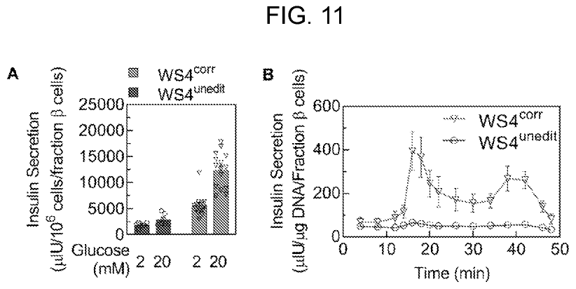

Lack of WFS1 can cause β cell dysfunction (27, 29, 32). Indeed, although both WS4^{unedit} and WS4^{corr} SC-β cells responded to glucose by secreting insulin in a static assay (FIG. 1F), only WS4^{corr} SC-β cells demonstrated robust first- and second-phase insulin secretion (FIG. 2E). WS4^{corr} SC-β cells secreted 8.9±2.1× more insulin in dynamic GSIS assays compared to WS4^{unedit} SC-β cells at peak (first phase) insulin secretion in 20 mM glucose. WS4^{corr} SC-β cells were functionally similar to non-diabetic SC-β cells and human islets (FIG. 2E), as previously reported (17). Even accounting for the differences in differentiation efficacy between WS4^{unedit} and WS4^{corr} iPSC lines, WS4^{corr} SC-β cells still showed superior static and dynamic GSIS (FIG. 11). Collectively, these data demonstrate that CRISPR/Cas9 correction of the WFS1 pathogenic variant in WS iPSCs increased SC-β cell differentiation efficacy and function, leading to greater and near islet-like dynamic insulin secretion in vitro in response to high glucose.

Gene-Corrected WS SC-β Cells Correct Blood Glucose in Mice with Pre-Existing Diabetes

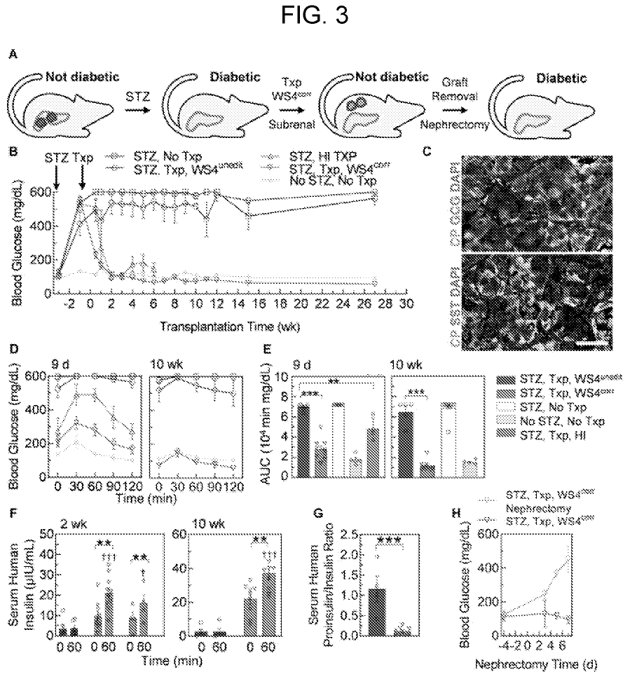

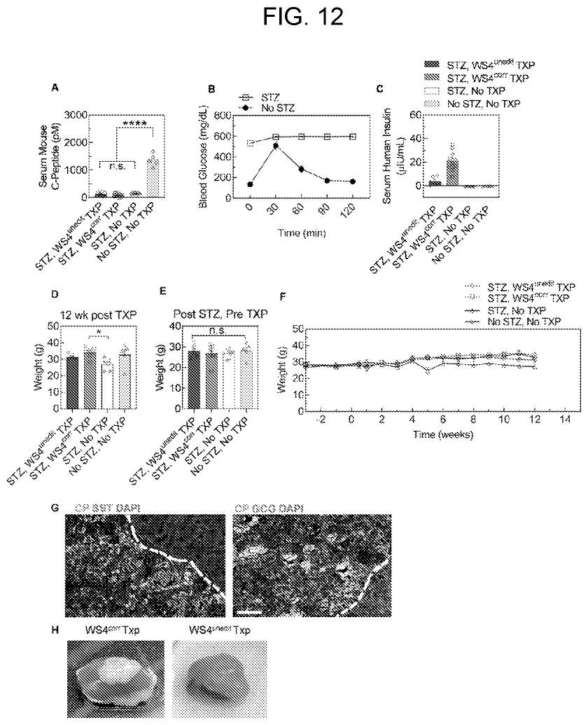

To evaluate the potential of corrected WS SC-β cells for cell replacement therapy, we transplanted 5×10⁶ WS4^{unedit} stage 6 cells, WS4^{corr} stage 6 cells, and cadaveric human islet cells under the kidney capsule of mice previously rendered diabetic by streptozotocin (STZ) injection to destroy endogenous mouse β cells (FIG. 3A, FIG. 12). Three sham mice spontaneously died, two at 14 wk and one at 25 wk, presumably due to their diabetes. After transplantation with WS4^{corr} SC-β cells, blood glucose dropped <200 mg/dL within 1 wk (FIG. 3B), with an average blood glucose concentration of 81±5 mg/dL from 2-27 wk. The grafts at two weeks contained cells mostly expressing C-peptide but there were also minority populations of glucagon+ and somatostatin+ cells (FIG. 3C, FIG. 12G). We performed glucose tolerance tests 9 d and 10 wk after transplantation, with WS4^{corr} SC-β cells demonstrating improved glucose tolerance compared to transplanted WS4^{unedit} cells and control sham mice (FIG. 3D-FIG. 3E). Transplanted human islets performed similarly albeit more slowly compared to WS4^{corr} cells in mice, resulting in normoglycemia at 2 wk, improved glucose tolerance at 9 days, and demonstrating in vivo GSIS at 2 wk post transplantation (FIG. 3B, FIG. 3D-FIG. 3E). For the 6-month observation period, both mice transplanted with WS4^{unedit} SC-β cells and sham mice were unable to achieve glycemic control, whereas the WS4^{corr} SC-β cells maintained blood glucose normalization. We collected mouse serum 2 and 10 wk after transplantation, and although all transplantation groups had detectable human insulin, WS4^{corr} had higher concentrations than WS4^{unedit} 5.8±1.5× and 14.4±2.8× at 2 and 10 wk respectively, and human islets had higher concentrations than WS4^{unedit} 4.4±1.5× at 2 wk, (FIG. 3F). This was consistent with our in vitro observations of increased insulin secretion and glucose responsiveness of WS4^{corr} compared to WS4^{unedit} SC-β cells in vitro. Serum insulin concentrations doubled from 2 to 10 wk in the WS4^{corr} SC-β cells, suggesting the SC-β cells were maturing in vivo (FIG. 3F). Mice transplanted with WS4^{unedit} SC-β cells also had a higher human proinsulin-to-insulin ratio in the serum, suggesting the presence of ER stress and problems with insulin processing (FIG. 3G). We performed nephrectomy of two live mice 12 wk after their transplantation with WS4^{corr} SC-β cells, which resulted in a return to hyperglycemia within 1 wk, indicating that prior glycemic control was due to the transplanted WS4^{corr} SC-β cells (FIG. 3H). We observed that kidneys receiving WS4^{unedit} cells contained undesirable large overgrowths that were not present in any kidneys with WS4^{corr} cells (FIG. 12H). The ability of WS4^{corr} SC-β cell-transplanted mice to reverse diabetes demonstrates that CRISPR/Cas9 correction of differentiated iPSCs from a patient with diabetes is a promising source of autologous β cells for diabetes therapy.

Single-Cell RNA Sequencing of Differentiated WS iPSCs Reveals SC-β Cell, Pancreatic Endocrine, and Non-Pancreatic Populations

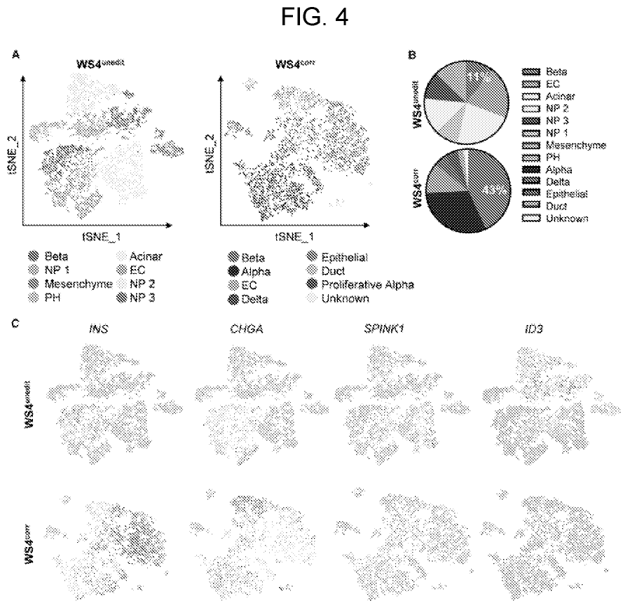

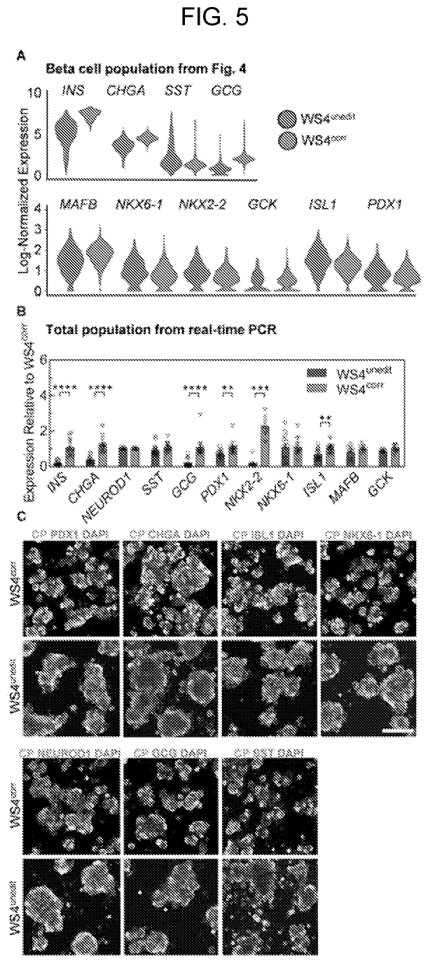

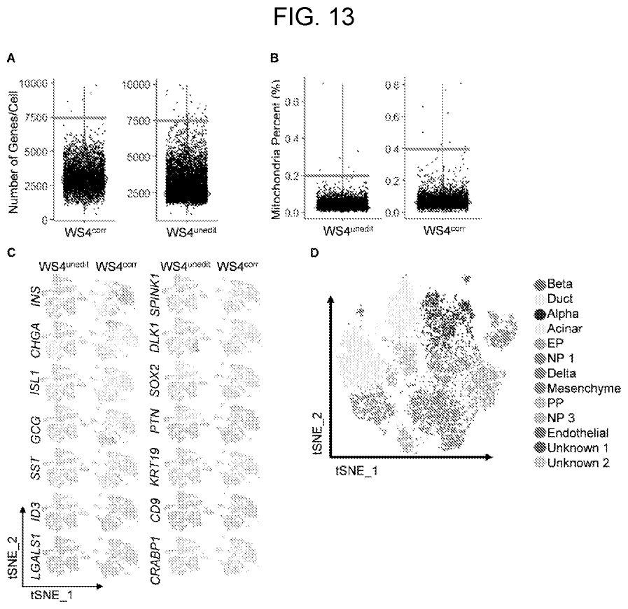

To compare SC-β cells generated from WS4^{corr} and WS4^{unedit} iPSCs while accounting for differences in differentiation efficacy (FIG. 2A), we performed scRNA-seq using the 10× Genomics platform. We sequenced a total of 7,215 and 3,640 stage 6 single cells differentiated from WS4^{unedit} and WS4^{corr} hiPSCs, respectively, averaging 50,996 reads per cell. After quality control (FIG. 13), an average of 2,861 genes were detected per cell. We used dimensionality reduction and unsupervised clustering to evaluate our scRNA-seq data and identified 8 cell populations in WS4^{corr} and WS4^{unedit} (FIG. 4A) based on the top upregulated genes in each cluster matched to exocrine and endocrine cell types distinguished in published pancreatic transcriptome data (TABLE 2) (43).

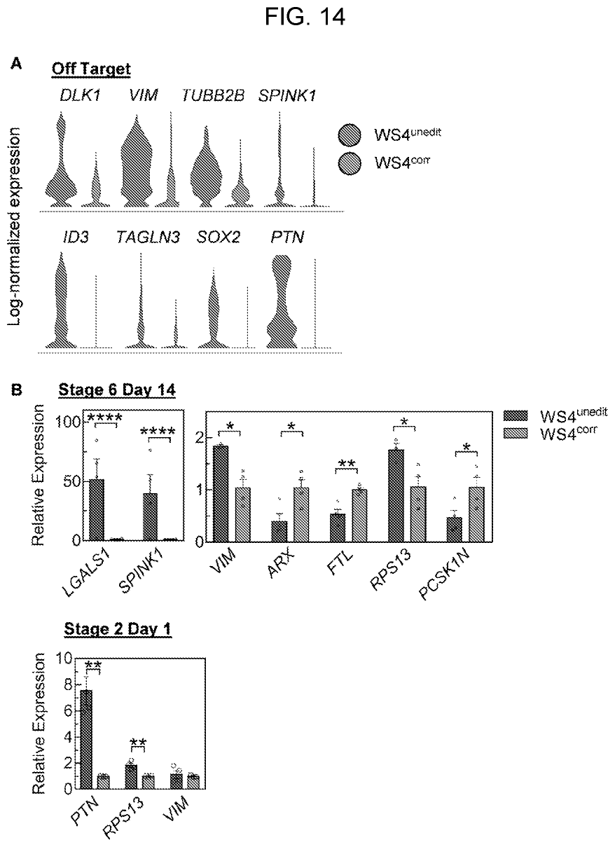

We identified SC-β cell populations in stage 6 cells differentiated from both WS4^{corr} and WS4^{unedit} iPSCs (FIG. 4A). Although SC-β cells were the largest population in differentiated WS4^{corr} cells (42%), they were a minority population in differentiated WS4^{unedit} cells (11%) (FIG. 4B). The vast majority (91%) of cells within WS4^{corr} stage 6 cells were identified as pancreatic endocrine (SC-β, SC-α, SC-δ), whereas pancreatic endocrine (SC-β, polyhormonal) were a minority (16.5%) for WS4^{unedit} cells (FIG. 4A-FIG. 4B). The majority of WS4^{unedit} stage 6 cells were either pancreatic exocrine or non-pancreatic cells (FIG. 4A-FIG. 4B), with many cells expressing non-3 cell markers such as SPINK1 and ID3 (FIG. 4C, FIG. 13C, FIG. 14A), suggesting that the WFS1 pathogenic variants carried by these cells caused misdirection of cell fate choice to off-targets with our differentiation protocol. Gene expression of the off-target markers was detectable as early as stage 2 with real-time PCR (FIG. 14B), suggesting the non-SC-β cell off-target cells were likely expanding as differentiation progressed to reduce the fraction of on-target pancreatic cells. The effects of these off-target populations on the function of the SC-β cell population, particularly in terms of transplantation efficacy, is unknown. Both WS4^{corr} and WS4^{unedit} pancreatic endocrine populations had cells with multiple detected hormones (insulin, glucagon, somatostatin) (FIG. 4C, FIG. 13C), as has been observed with endocrine stem cell differentiations and human islets (44-46). Taken together, scRNA-seq enabled identification of SC-β cells from WS4^{corr} and WS4^{unedit} iPSCs differentiated to stage 6, in addition to several unexpected off-targets from WS4^{unedit} cells, illustrating the greatly improved SC-β cell differentiation efficacy enabled by CRISPR/Cas9 correction in WS iPSCs.

CRISPR/Cas9 Gene Editing Modulated SC-N Cell Gene Expression

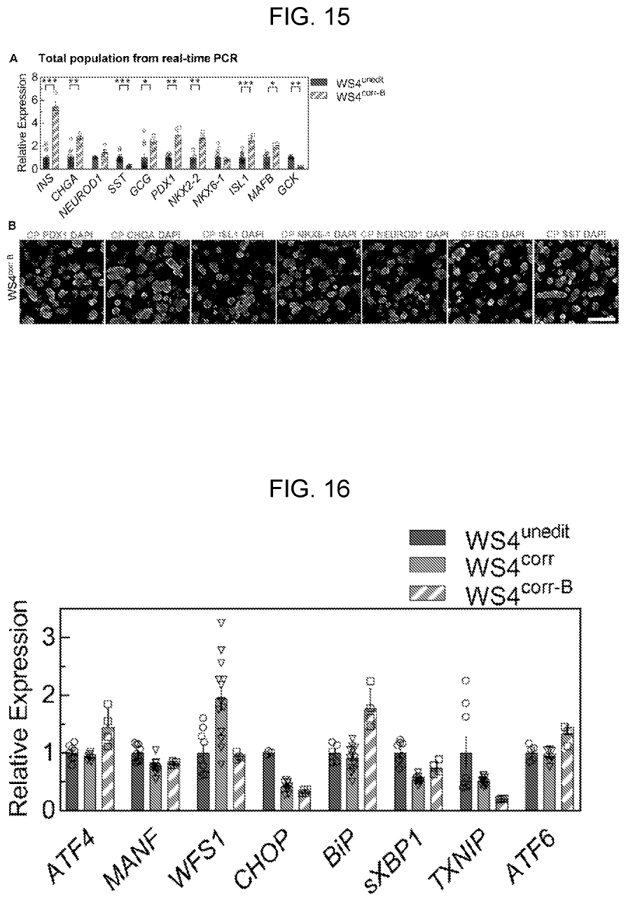

scRNA-seq enabled investigation of the transcriptome specifically in SC-β cells from the heterogeneous stage 6 cell population in both WS4^{corr} and WS4^{unedit} lines. This eliminated dilution effects on analysis of the bulk population that could occur due to the presence of non-SC-β cell populations and markers expressed by multiple cell types, such as NKX6-1 expressed by both pancreatic progenitors and β cells. We first evaluated expression of β cell markers within the SC-β cell population, as our prior work had shown that inflammatory stress could reduce key β cell proteins (18). INS, CHGA, and GCG expression within SC-β cells was reduced, whereas SST expression increased, in WS4^{unedit} compared to WS4^{corr} SC-β cells (FIG. 5A, TABLE 3A). Additionally, gene expression of transcription factors important to β cell identity (NKX6-1, ISL1, PDX1) and GCK, a necessary β cell functional gene, were similar in WS4^{unedit} compared to WS4^{corr} SC-β cells. Real-time PCR measurements of the total stage 6 populations for WS4^{unedit}, WS4^{corr}, and WS4^{corr-B} cell lines showed similar reductions in INS and CHGA transcripts (FIG. 5B, FIG. 15A). Some β cell markers such as NKX2-2 were lower in WS4^{unedit} compared to WS4^{corr} and WS4^{corr-B} stage 6 cells (FIG. 5B, FIG. 15A), however this was likely due to lower differentiation yields of WS4^{unedit} SC-β cells. Immunostaining confirmed that C-peptide+ cells co-expressed many β cell proteins, including PDX1, CHGA, ISL1, NKX6-1, and NEUROD1 (FIG. 5C, FIG. 15B). These data indicate that SC-β cells with CRISPR/Cas9 correction of WFS1 were mostly similar in terms of p cell marker expression compared to unedited patient cells except notably for INS, which showed greatly reduced transcript abundance. Furthermore, both WS4^{unedit} and WS4^{corr} SC-β cells co-expressed many β cell protein markers, similar to prior reports on non-diabetic SC-β cells (17, 23).

CRISPR/Cas9 Gene Editing Decreased SC-β Cell ER and Mitochondrial Stress

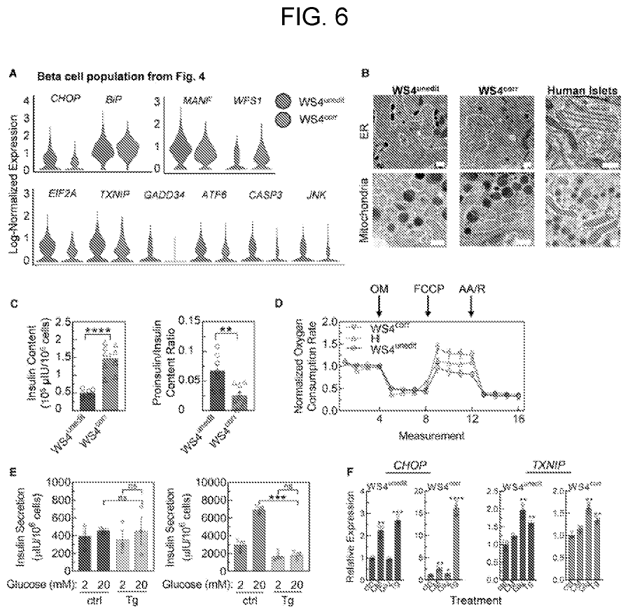

As pathogenic variants in WFS1 cause ER stress in β cells (27-29), we next evaluated the expression of stress markers within WS4^{corr} and WS4^{unedit} SC-β cells using our scRNA-seq data. ER stress can affect many pathways in cell metabolism, including activation of the unfolded protein response, mitochondrial stress, and apoptosis. We observed increased expression of many adaptive ER stress (eIF2a, MANF), terminal ER stress (CHOP), mitochondrial stress (TXNIP), and apoptotic (CASP3) markers within WS4^{unedit} compared to WS4^{corr} SC-β cells (FIG. 6A, TABLE 3B). WFS1 expression was elevated in WS4^{corr} compared to WS4^{unedit} SC-β cells (FIG. 6A), consistent with mouse and immortalized cell line studies (28, 29) and transcript and protein measurements of the entire stage 6 population. Discerning definitive trends with real-time PCR analysis for a wide range of markers in the bulk stage 6 population was difficult (FIG. 16A), likely due to the dilution effects of differing SC-β cell differentiation efficacy. Adaptive ER (eIF2a, MANF) and mitochondrial (TXNIP) stress markers are expressed across most cell types but are typically more highly expressed in high protein-producing and glucose-responsive cells (47), confounding study in this system.

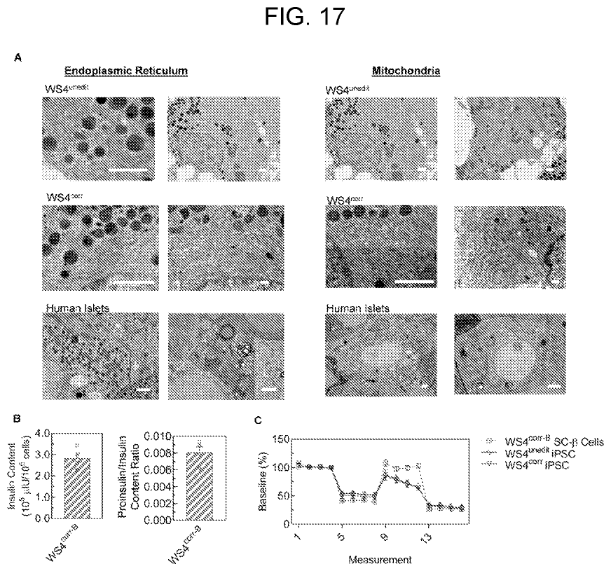

To investigate the failure of WS4^{unedit} SC-β cells to properly function, we used transmission electron microscopy (TEM) to observe the morphology of SC-β cell ER and mitochondria (FIG. 6B, FIG. 17A). ER and mitochondria for WS4^{corr} cells appeared normal and healthy compared to human islets. WS4^{unedit} cell ER were swollen and expanded in the cytoplasm, measuring 4.9±1.2× (p<0.05) and 3.0±0.8× (p<0.01) larger than WS4^{corr} cell and human islets, respectively, which can occur in response to ER stress (32). The WS4^{unedit} mitochondria appear fragmented and undergoing fission, measuring 1.9±0.3× (p<0.001) and 2.0±0.3× (p<0.0001) smaller than WS4^{corr} and human islets, respectively, consistent with mitochondria exhibiting dysfunction in response to chronic ER stress (48-50). Our SC-β cells contained a variety of granules in various stages of maturation, as previously reported (15, 16, 51). As the ER is critical for proper insulin processing, we measured the insulin content and proinsulin-to-insulin ratio of stage 6 cells. We observed that WS4^{unedit} cells had lower insulin content and a higher proinsulin-to-insulin ratio than WS4^{corr} cells (FIG. 6C, FIG. 17B). Combined with the observed increases in unfolded protein response markers and unhealthy ER morphology shown by TEM, these data support WS4^{unedit} SC-β cells have defects in insulin processing that persist for months after transplantation. Mitochondria are critical for proper glucose sensing, and therefore we assessed their function by measuring the oxygen consumption rate (OCR) after injection of interrogating compounds (52). We found that WS4^{unedit} cells had a lower OCR after injection with carbonyl cyanide p-trifluoromethoxyphenylhydrazone (FCCP), a mitochondrial oxidative phosphorylation uncoupler, compared to WS4^{corr} cells (p<0.0001; two-way ANOVA), WS4^{corr-B}, and human islets (p<0.001; two-way ANOVA) (FIG. 6D, FIG. 17C). There was no difference in OCR when interrogating the cells with Oligomycin or Antimycin A (AA) and rotenone injections, which inhibits ATP synthase and mitochondrial electron transport chain, respectively. This indicates WS4^{unedit} cells have reduced maximal mitochondrial respiratory capacity, as seen in other reports of monogenic diabetes (53) and consistent with stressed and unhealthy mitochondria (FIG. 6A-FIG. 6B). The difference was not observed with undifferentiated WS4^{corr} and WS4^{unedit} iPSCs (FIG. 17C), indicating this phenotype was restricted to certain cell types, such as SC-β cells. Taken together, SC-β cells harboring pathogenic variants in WFS1 displayed ER and mitochondrial dysfunction, which was restored with CRISPR/Cas9 gene correction.

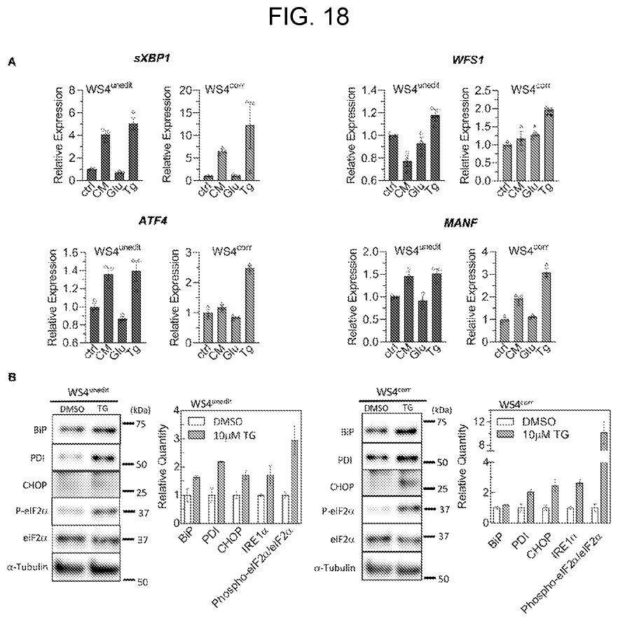

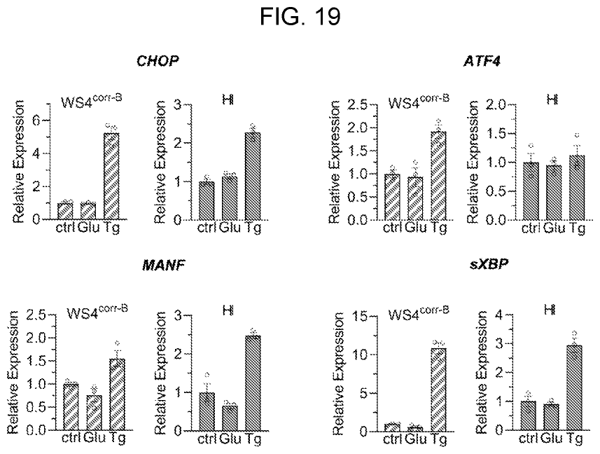

To further investigate the effects of cellular stress on WS SC-β cells, we treated WS4^{unedit} and WS4^{corr} stage 6 cells with thapsigargin, an inhibitor of sarco/endoplasmic reticulum Ca²⁺ ATPase (SERCA), to induce ER stress. We performed static GSIS and whereas insulin secretion was not affected in WS4^{unedit} cells, likely because these cells already had low secretion and function, insulin secretion in WS4^{corr} cells was strongly negatively affected by this compound, decreasing insulin secretion by 3.9±0.7× and eliminating glucose-responsiveness (FIG. 6E). Using bulk stage 6 population analysis with real-time PCR and western blot, we also observed that both WS4^{unedit} and WS4^{corr} cells responded to thapsigargin treatment by increasing known stress markers (FIG. 6F, FIG. 18, FIG. 19). These data indicate that the enhanced functional and reduced ER stress transcriptional benefits of CRISPR/Cas9 gene correction can be reversed with chemical-induced ER stress.

Tables

TABLE 1
CRISPR sequences
WS4

| | |
|---|---|
| Sp14 gRNA | 5′ GACCATGGAGCTCCACGTCA(GGG) 3′ (SEQ ID NO: 1) |
| ssODN | 5′ ACCAGCAGAACAGCACGATGGCCGTGAGCCACACCAGGATGAGCTTGA |
| (anti-sense) | CCATGGAGCTCCGCGTCAGGGACTTCACCATCCCCACCACAGAGAAGCTG |
| | GCCTTGGTCCACCAGCGCAACAGCA 3′. (SEQ ID NO: 2) |

WS13

| | |
|---|---|
| Sp11 gRNA | 5′ CATTCTCCAGCGCTCCGCCA(CGG) 3′ (SEQ ID NO: 3) |
| repair ssODN | 5′ GGCAGCCCTGGTCATGTACTGGAAGCTCAACCCCAAGAAGAAGAA |
| (sense) | GCAGGTGGCCGTGGCAGAGCTGCTGGAGAATGTCGGCCAGGTCAAC |
| | GAGCACGGTGCGAGGATTCACCCTGGGCACCAGCCT 3′. |
| | (SEQ ID NO: 4) |

TABLE 2
Additional analysis of WS4^{corr} and WS4^{unedit} SC-β cell scRNA-seq and
population upregulated genes.

| A. | WS4^{corr} | WS4^{unedit} |
|---|---|---|
| Beta | 43.10% | 10.50% |
| Alpha | 29.50% | — |
| EC | 11.90% | 20.40% |
| Delta | 5.00% | — |
| Epithelial | 4.90% | — |
| Duct | 2.30% | — |
| Prolif. | 1.50% | — |
| Alpha | | |
| Acinar | 1.90% | — |
| NP 1 | — | 14.10% |
| NP 2 | — | 10.80% |
| NP 3 | — | 9.10% |
| Mesenchyme | — | 6.70% |
| PH | — | 6.00% |
| Unknown | | 22.50% |

B.
WS4^{unedit}

| Acinar | EC | NP 1 | NP 2 | NP 3 | Beta | Mesenchyme | PH |
|---|---|---|---|---|---|---|---|
| DLK1 | CHGA | PTN | TUBA1B | ID3 | INS | MGP | TAGLN3 |
| SPINK1 | PCSKIN | IFITM3 | HMGB2 | ID1 | TTR | LGALS1 | TUBB2B |
| CTRB2 | CRYBA2 | SOST | HMGN2 | PTN | PCSKIN | COL3A1 | CRABP1 |
| CLPS | FEV | TTYH1 | H2AFZ | VIM | SST | VIM | TUBA1A |
| B2M | IAPP | NLRP1 | HIST1H4C | TUBA1A | GCG | CRABP1 | HES6 |

WS4^{corr}

| | | | | | | Prolif | |
|---|---|---|---|---|---|---|---|
| Beta | Alpha | EC | Delta | Epithelial | Duct | Alpha | Acinar |
| ERO1LB | GCG | TPH1 | CRH | LGALS1 | ANXA1 | H2AFZ | RPLPO |
| INS | TTR | FEV | SST | VIM | SPRR1B | HMGN2 | RPS3A |
| IAPP | ALDH1A1 | DDC | H3F3B | RPL10 | KRT17 | PTMA | RPL3 |
| C1QL1 | BTG1 | CHGA | MALAT1 | RPL15 | KRT19 | STMN1 | EEF1A1 |
| ETL | PCSKIN | CRYBA2 | GHRL | EEF1A1 | S100A11 | TUBA1B | FTL |
| — | — | — | — | — | — | — | |
| C. | — | — | — | — | — | — | |
| Beta | Duct | Alpha | Acinar | EC | NP 1 | Delta | |

| | | | | | | |
|---|---|---|---|---|---|---|
| INS | RSPO1 | GCG | RPS18 | TPH1 | CENPM | RTN1 |
| ERO1LB | RSPO3 | ARX | RPL12 | COL5A2 | CDK1 | MIR7-3HG |
| ACVR1C | SOX2 | SPTSSB | RPL11 | SLC18A1 | PRC1 | ARHGDIG |
| PCP4 | ZIC1 | TTR | RPS3 | FEV | NUSAP1 | STMN2 |
| HPCA | RMST | ALDH1A1 | GNB2L1 | MGLL | BIRC5 | BEX2 |
| PDX1 | NTRK2 | BTG1 | RPS14 | SYT13 | CKAP2L | STMN4 |

| Mesenchyme | PP | NP 2 | Endothelial | Unknown 1 | Unknown 2 | — |
|---|---|---|---|---|---|---|
| LGALS1 | TOP2A | TAGLN3 | CD9 | RPL41 | MT-CO3 | — |
| MGP | CKS1B | TUBB2B | TTYH1 | RPS13 | MALAT1 | — |
| CRABP1 | RRM2 | TUBA1A | LGALS3 | RPL35A | MT-ND4 | — |
| PTN | HMGB2 | MIAT | ANXA1 | RPS12 | MT-CYB | — |
| COL1A2 | MAD2L1 | CRMP1 | ANXA3 | RPL21 | MT-CO1 | — |
| COL3A1 | SMC4 | MAP1B | ANXA2 | RPL9 | MT-CO2 | — |

This table is associated with FIG. 4. (A) Percentage of sequenced cells present in each population shown in FIG. 3. (B) Top 5 enriched genes for each population for WS4unedit and WS4corr stage 6 cells separately. (C) Top 5 enriched genes for WS4unedit and WS4corr stage 6 cells combined.

TABLE 3
Log fold change values between WS4^{corr} and WS4^{unedit} SC-ß cells for
markers in FIG. 5A and FIG. 6A.

| | | | | | | higher |
|---|---|---|---|---|---|---|
| | | | | | | expression |
| A. | p_val | avg_logFC | pct.1 | pct.2 | p_val_adj | in: |
| INS | 3.30E−222 | 1.701845 | 1 | 0.995 | 8.68E−218 | corr |
| CHGA | 4.82E−147 | 0.766731 | 1 | 1 | 1.27E−142 | corr |
| SST | 3.80E−42 | −1.39873 | 0.961 | 0.966 | 9.99E−38 | unedit |
| GCG | 4.71E−232 | 2.042099 | 0.997 | 0.613 | 1.24E−227 | corr |
| MAFB | 7.32E−44 | 0.330119 | 0.993 | 0.881 | 1.92E−39 | corr |
| NKX6-1 | 5.44E−09 | −0.22229 | 0.762 | 0.77 | 0.000143 | unedit |
| NKX2-2 | 1.76E−13 | −0.23707 | 0.703 | 0.761 | 4.61E−09 | unedit |
| GCK | 0.003996 | 0.015245 | 0.494 | 0.382 | 1 | corr |
| ISL1 | 1.30E−14 | −0.27081 | 0.936 | 0.931 | 3.42E−10 | unedit |
| PDX1 | 0.866413 | −0.05035 | 0.789 | 0.694 | 1 | unedit |
| | | | | | | higher |
| | | | | | | expression |
| B. | p val | avg logFC | pct.1 | pct.2 | p_val_adj | in: |
| DDIT3/ | 2.61E−08 | −0.21787 | 0.322 | 0.408 | 0.000687 | unedit |
| CHOP | | | | | | |
| HSPA5/ | 3.17E−15 | 0.153105 | 0.954 | 0.839 | 8.34E−11 | corr |
| BIP | | | | | | |
| MANF | 0.000118 | −0.16794 | 0.758 | 0.708 | 1 | unedit |
| WFS1 | 3.97E−22 | 0.196655 | 0.51 | 0.252 | 1.04E−17 | corr |
| EIF2A | 3.22E−12 | −0.18554 | 0,383 | 0.492 | 8.47E−08 | unedit |
| TXNIP | 0.048835 | −0.08196 | 0.477 | 0.462 | 1 | unedit |
| GADD34 | 7.63E−14 | −0.14527 | 0.111 | 0.235 | 2.00E−09 | unedit |
| ATF6B | 2.05E−14 | −0.22753 | 0.426 | 0.541 | 5.39E−10 | unedit |
| CASP3 | 8.94E−11 | −0.14719 | 0.169 | 0.286 | 2.35E−06 | unedit |
| MAPK8 | 2.66E−05 | −0.08681 | 0.176 | 0.249 | 0.699975 | unedit |

(A) Log fold change values between WS4^{corr} and WS4^{unedit} SC-B cells, detailing which cell type has greater expression according to avg_logFC. Nonadjusted (p_val) and adjusted (p_val_adj) p-values, and percentage of cells positive for measured gene in WS4^{corr} (pct.1) and WS4^{unedit} (pct.2) SC-B cells calculated for violin plots in FIG. 5A.
(B) Log fold change values between WS4^{corr} and WS4^{unedit} SC-B cells, detailing which cell type has greater expression according to avg_logFC. nonadjusted (p_val) and adjusted (p_val_adj) p-values, and percentage of cells positive for measured gene in WS4^{corr} (pct.1) and WS4^{unedit} (pct.2) SC-B cells calculated for violin plots in FIG. 6A.

TABLE 4
Differentiation protocol

| | Day | Media | Factor | Final Concentration | |
|---|---|---|---|---|---|
| Stage 0 | 1 | mTeSR1 | Y27632 | 10 | μM |
| — | — | — | — | — | |
| Stage 1 | 2 | BE1 | Activin A | 100 | ng/mL |
| — | — | — | CHIR99021 | 3 | μM |
| Stage 1 | 3 | BE1 | Activin A | 100 | ng/mL |
| Stage 1 | 4 | BE1 | Activin A | 100 | ng/mL |
| Stage 1 | 5 | BE1 | Activin A | 100 | ng/mL |
| — | — | — | — | — | |
| Stage 2 | 6 | BE2 | KGF | 50 | ng/mL |
| — | 7 | BE2 | KGF | 50 | ng/mL |
| — | — | — | — | — | |
| Stage 3 | 8 | BE3 | LDN193189 | 200 | nM |
| — | — | — | KGF | 50 | ng/mL |
| — | — | — | SANT1 | 0.25 | μM |
| — | — | — | RA | 2 | μM |
| — | — | — | TPPB | 500 | nM |
| Stage 3 | 9 | BE3 | LDN193189 | 200 | nM |
| — | — | — | KGF | 50 | ng/mL |
| — | — | — | SANT1 | 0.25 | μM |
| — | — | — | RA | 2 | μM |
| — | — | — | TPPB | 500 | nM |
| — | — | — | — | — | |
| Stage 4 | 10 | BE3 | KGF | 50 | ng/mL |
| — | — | — | SANT1 | 0.25 | μM |
| — | — | — | RA | 0.1 | μM |
| — | — | — | TPPB | 500 | nM |
| — | — | — | LDN193189 | 200 | nM |
| — | 11 | BE3 | KGF | 50 | ng/mL |
| — | — | — | SANT1 | 0.25 | μM |
| — | — | — | RA | 0.1 | μM |
| — | — | — | TPPB | 500 | nM |
| — | — | — | LDN193189 | 200 | nM |
| — | 12 | BE3 | KGF | 50 | ng/mL |
| — | — | — | SANT1 | 0.25 | μM |
| — | — | — | RA | 0.1 | μM |
| — | — | — | TPPB | 500 | nM |
| — | — | — | LDN193189 | 200 | nM |
| — | 13 | BE3 | KGF | 50 | ng/mL |
| — | — | — | SANT1 | 0.25 | μM |
| — | — | — | RA | 0.1 | μM |
| — | — | — | TPPB | 500 | nM |
| — | — | — | LDN193189 | 200 | nM |
| — | — | — | — | — | |
| Stage 5 | 14 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Latrunculin A | 1 | μM |
| — | — | — | Betacellulin | 20 | ng/mL |
| — | 15 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Betacellulin | 20 | ng/mL |
| — | 16 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Betacellulin | 20 | ng/mL |
| — | 17 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Betacellulin | 20 | ng/mL |
| — | 18 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Betacellulin | 20 | ng/mL |
| — | 19 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Betacellulin | 20 | ng/mL |
| — | 20 | S5 | SANT1 | 0.25 | μM |
| — | — | — | RA | 0.1 | μM |
| — | — | — | XXI | 1 | μM |
| — | — | — | Alk5i | 10 | μM |
| — | — | — | T3 | 1 | μM |
| — | — | — | Betacellulin | 20 | ng/mL |

| | | | | |
|---|---|---|---|---|
| — | — | — | — | — |

| | |
|---|---|
| Stage 6 | Feed ESFM every other day |

TABLE 5
Differentiation factor list

| Compound | Company |
|---|---|
| Activin A | R&D Systems |
| CHIR99021 | Stemgent |
| KGF | Peprotech |
| LDN193189 | Reprocell |
| PdBU | MilliporeSigma |
| Retinoic Acid | MilliporeSigma |
| SANT1 | MilliporeSigma |
| Y27632 | Abcam |
| Latrunculin A | Cayman Chemical |
| Alk5 inhibitor type II | Enzo Life Sciences |
| Betacellulin | R&D Systems |
| T3 | Biosciences |
| XXI | MilliporeSigma |
| TPPB | Tocris |

TABLE 6
Media and buffer formulations
Differentiation Media

| Reagent | BE1 | BE2 | BE3 | S5 | ESFM | Company |
|---|---|---|---|---|---|---|
| MCDB131 | 500 mL | 500 mL | 500 mL | 500 mL | 500 mL | Cellgro |
| Glucose | 0.8 g | 0.4 g | 0.22 g | 1.8 g | 0.23 g | MilliporeSigma |
| NaHCO3 | 0.587 g | 0.587 g | 0.877 g | 0.877 g | N/A | MilliporeSigma |
| FAF-BSA | 0.5 g | 0.5 g | 10 g | 10 g | 10.5 g | Proliant |
| ITS-X | N/A | N/A | 2.5 mL | 2.5 mL | N/A | Invitrogen |
| Glutamax | 5 mL | 5 mL | 5 mL | 5 mL | 5.2 mL | Invitrogen |
| Vitamin C | N/A | 22 mg | 22 mg | 22 mg | N/A | MilliporeSigma |
| Heparin | N/A | N/A | N/A | 5 mg | 10 ug/mL | MilliporeSigma |
| Pen/Strep | N/A | N/A | N/A | 5 mL | 5.2 mL | Cellgro |
| Non-Essential Amino | N/A | N/A | N/A | N/A | 5.2 mL | Corning |
| Acids | | | | | | |
| ZnSO4 | N/A | N/A | N/A | N/A | 1 uM | MilliporeSigma |
| Trace Elements A | N/A | N/A | N/A | N/A | 523 UL | Corning |
| Trace Elements B | N/A | N/A | N/A | N/A | 523 UL | Corning |

Islet Culture Media

| Reagent | Volume | Company |
|---|---|---|
| CMRL Supplemented | 500 mL | Mediatech |
| Fetal Bovine Serum | 55.5 mL | GE Healthcare |

KRB Buffer (in deionized water)

| Reagent | Concentration |
|---|---|
| NaCl | 128 mM |
| KCI | 5 mM |
| CaCI2 | 2.7 mM |
| MgSO4 | 1.2 mM |
| Na2HPO4 | 1 mM |
| KH2PO4 | 1.2 mM |
| NaHCO3 | 5 mM |
| HEPES | 10 mM |
| BSA | 0.10% |

Immunostaining Solution

| Reagent | Concentration | Company |
|---|---|---|
| PBS | | Fisher |
| Triton X | 0.10% | Acros Organics |
| Donkey serum | 5% | Jackson Immunoresearch; |

TABLE 7
Antibody list

| | | Dilution - | Dilution - |
|---|---|---|---|
| Antibody Target | Company | Imaging | Blotting |
| C-peptide | DSHB | 1:300 | N/A |
| NKX6-1 | DSHB | 1:100 | N/A |
| Glucagon | Abcam | 1:300 | N/A |
| PDX1 | R&D Systems | 1:300 | N/A |
| PAX6 | BDBiosciences | 1:300 | N/A |
| CHGA | Abcam | 1:1000 | N/A |
| ISL1 | DSHB | 1:300 | N/A |
| GCG | Cell Marque | 1:2 | N/A |
| WFS1 | ProteinTech | 1:300 | 1:2000 |
| rabbit-anti-BiP | Cell Signaling | N/A | 1:1000 |
| mouse-anti-PDI | Enzo | N/A | 1:1000 |
| mouse-anti-CHOP | Cell Signaling | N/A | 1:1000 |
| rabbit-anti-Phospho elF2α | Cell Signaling | N/A | 1:1000 |
| rabbit-anti-elF2α | Cell Signaling | N/A | 1:1000 |
| rabbit-anti-αTubulin | Cell Signaling | N/A | 1:2000 |
| HRP-linked anti-rabbit IgG | Cell Signaling | N/A | 1:2000 |
| HRP-linked anti-mouse IgG | Cell Signaling | N/A | 1:2000 |
| anti-rat-alexa fluor 488 | Invitrogen | 1:300 | N/A |
| anti-mouse-alexa fluor 594 | Invitrogen | 1:300 | N/A |
| anti-rabbit-alexa fluor 594 | Invitrogen | 1:300 | N/A |
| anti-goat-alexa fluor 594 | Invitrogen | 1:300 | N/A |
| anti-rat-PE | Jackson | 1:300 | N/A |
| | Immuneresearch | | |

DSHB = Developmental
Studies Hybridoma Bank
HRP = Horseradish peroxidase

TABLE 8
Primers used for real-time PCR

| Gene | | |
|---|---|---|
| name | Forward Primer Sequence | Reverse Primer Sequence |
| INS | CAATGCCACGCTTCTGC (SEQ ID | TTCTACACACCCAAGACCCG (SEQ |
| | NO: 5) | ID NO: 6) |
| CHGA | TGACCTCAACGATGCATTTC (SEQ | CTGTCCTGGCTCTTCTGCTC (SEQ |
| | ID NO: 7) | ID NO: 8) |
| NEUROD1 | ATGCCCGGAACTTTTTCTTT (SEQ | CATAGAGAACGTGGCAGCAA (SEQ |
| | ID NO: 9) | ID NO: 10) |
| SST | TGGGTTCAGACAGCAGCTC (SEQ | CCCAGACTCCGTCAGTTTCT (SEQ |
| | ID NO: 11) | ID NO: 12) |
| GCG | AGCTGCCTTGTACCAGCATT (SEQ | TGCTCTCTCTTCACCTGCTCT (SEQ |
| | ID NO: 13) | ID NO: 14) |
| PDX1 | CGTCCGCTTGTTCTCCTC (SEQ ID | CCTTTCCCATGGATGAAGTC (SEQ |
| | NO: 15) | ID NO: 16) |
| NKX2-2 | GGAGCTTGAGTCCTGAGGG (SEQ | TCTACGACAGCAGCGACAAC (SEQ |
| | ID NO: 17) | ID NO: 18) |
| NKX6-1 | CCGAGTCCTGCTTCTTCTTG (SEQ | ATTCGTTGGGGATGACAGAG (SEQ |
| | ID NO: 19) | ID NO: 20) |
| ISL1 | TCACGAAGTCGTTCTTGCTG (SEQ | CATGCTTTGTTAGGGATGGG (SEQ |
| | ID NO: 21) | ID NO: 22) |
| ARMET | GGCCAGAGGCTTTGATACCT (SEQ | GCAAGAGGCAAAGAGAATCG (SEQ |
| | ID NO: 23) | ID NO: 24) |
| WFS1 | AAGGGGATTTCCATGTCTCC (SEQ | CCTGGTGTTAGAGACGCAGC (SEQ |
| | ID NO: 25) | ID NO: 26) |
| DDIT3 | TGGATCAGTCTGGAAAAGCA (SEQ | AGCCAAAATCAGAGCTGGAA (SEQ |
| | ID NO: 27) | ID NO: 28) |
| HSPA5 | TGTCTTTTGTCAGGGGTCTTT (SEQ | CACAGTGGTGCCTACCAAGA (SEQ |
| | ID NO: 29) | ID NO: 30) |
| sXBP1 | ATCCATGGGGAGATGTTCTGG | CTGAGTCCGAATCAGGTGCAG |
| | (SEQ ID NO: 31) | (SEQ ID NO: 32) |
| TXNIP | AGGAAGCTCAAAGCCGAACT (SEQ | ACGCTTCTTCTGGAAGACCA (SEQ |
| | ID NO: 33) | ID NO: 34) |
| TBP | GCCATAAGGCATCATTGGAC (SEQ | AACAACAGCCTGCCACCTTA (SEQ |
| | ID NO: 35) | ID NO: 36) |
| GUSB | CGTCCCACCTAGAATCTGCT (SEQ | TTGCTCACAAAGGTCACAGG (SEQ |
| | ID NO: 37) | ID NO: 38) |
| GCK | ATGCTGGACGACAGAGCC (SEQ ID | CCTTCTTCAGGTCCTCCTCC (SEQ |
| | NO: 39) | ID NO: 40) |
| MAFB | CATAGAGAACGTGGCAGCAA | ATGCCCGGAACTTTTTCTTT (SEQ ID |
| | (SEQ ID NO: 41) | NO: 42) |
| ATF4 | GCATGGTTTCCAGGTCATCT (SEQ | AGTCCCTCCAACAACAGCAA (SEQ |
| | ID NO: 43) | ID NO: 44) |
| ATF6 | TGGTAGCTGGTAACAGCAGG | CAGGAACTCAGGGAGTGAGC (SEQ |
| | (SEQ ID NO: 45) | ID NO: 46) |
| RSPO1 | CCTGGTGATTCAGGTGGGAGT | GGTTGATTGCCTCGACACCA (SEQ |
| | (SEQ ID NO: 47) | ID NO: 48) |
| ARX | TGGAAACAGAGGACCAGCAC | GTTGGAGTTGGAGCGAGGTT (SEQ |
| | (SEQ ID NO: 49) | ID NO: 50) |
| RPL12 | CCAAGGTGCAACTTCCTTCG (SEQ | GATCTCGTTGGGGTCGAACT (SEQ |
| | ID NO: 51) | ID NO: 52) |
| COL5A2 | TCCCAAAAGACAAGCCATGC | AACCACTGACATGACAAAAGCG |
| | (SEQ ID NO: 53) | (SEQ ID NO: 54) |
| COL3A1 | CGCCCTCCTAATGGTCAAGG | TTCTGAGGACCAGTAGGGCA (SEQ |
| | (SEQ ID NO: 55) | ID NO: 56) |
| CRABP1 | ATCCACTGCACGCAAACTCT (SEQ | CTGCCTTCACTCTCGGACAT (SEQ |
| | ID NO: 57) | ID NO: 58) |
| HES6 | AAGCCCCTGGTGGAGAAGAA | CTTCGGCGTTCTCCAGCTT (SEQ ID |
| | (SEQ ID NO: 59) | NO: 60) |
| CDKN3 | AAGCCGCCCAGTTCAATACA (SEQ | CCTGGAAGAGCACATAAACCGA |
| | ID NO: 61) | (SEQ ID NO: 62) |
| LGALS1 | ACGCTAAGAGCTTCGTGCTG (SEQ | TGAAGCGAGGGTTGAAGTGC (SEQ |
| | ID NO: 63) | ID NO: 64) |
| MGP | TTTGTGTTATGAATCACATGAAAGC | GGCTTCCCTATTGAGCTCGT (SEQ |
| | (SEQ ID NO: 65) | ID NO: 66) |
| DLK1 | CCGAGTTCACAGGTCTCACC (SEQ | GGTCTCGCACTTGTTGAGGA (SEQ |
| | ID NO: 67) | ID NO: 68) |
| NESTIN | GCCATAGAGGGCAAAGTGGT | GTTCCCCTAGAGACCTCCGT (SEQ |
| | (SEQ ID NO: 69) | ID NO: 70) |
| SMC4 | TTGATTGATCCTCAGGCACGTC | AGTGGCAAGAAGGCAGGACT (SEQ |
| | (SEQ ID NO: 71) | ID NO: 72) |
| TAGLN3 | AGAAGGGGGAAGAAACGTCC | CCCTGTTAGCCATCTAATCAAGTCT |
| | (SEQ ID NO: 73) | (SEQ ID NO: 74) |
| TUBB2B | GGGTCTCTGGTGCTCTTCAC (SEQ | AGAGGACACCATTCCGACAC (SEQ |
| | ID NO: 75) | ID NO: 76) |
| HMGB2 | CTCCGGCTTCCCCTCTCC (SEQ ID | GCGTACGAGGACATTTTGCC (SEQ |
| | NO: 77) | ID NO: 78) |
| RPL41 | GCGCTCCATTAAATAGCCGT (SEQ | CTCTCATGGCGCAGAGGTTT (SEQ |
| | ID NO: 79) | ID NO: 80) |
| RPS13 | CTGACGACGTGAAGGAGCAG | CAAACGGTGAATCCGGCTCT (SEQ |
| | (SEQ ID NO: 81) | ID NO: 82) |
| MT-CO3 | GATTTCACTTCCACTCCATAACG | CTTCTAGGGGATTTAGCGGG (SEQ |
| | (SEQ ID NO: 83) | ID NO: 84) |
| MALAT1 | GAATTGCGTCATTTAAAGCCTAGTT | GTTTCATCCTACCACTCCCAATTAAT |
| | (SEQ ID NO: 85) | (SEQ ID NO: 86) |
| PTN | TCTCATGGCCTCTTGGTTCAA | TCGAGAGCTGATTGGGAAGC (SEQ |
| | (SEQ ID NO: 87) | ID NO: 88) |
| SOST | AAATCACATCCGCCCCAACT (SEQ | GGCGGTGTCTCAAAAGGGAT (SEQ |
| | ID NO: 89) | ID NO: 90) |
| VIM | AAAAGTCCGCACATTCGAGC (SEQ | CGCTGCTAGTTCTCAGTGCT (SEQ |
| | ID NO: 91) | ID NO: 92) |
| SPINK1 | TGGCTCCTTTCACCTTTCTTACAC | CTGCGTCCAGAGGTCAGTTG (SEQ |
| | (SEQ ID NO: 93) | ID NO: 94) |
| PCSK1N | CGTCCAGAGCAACTTACCCC (SEQ | CATGTTTATTGTGGGGCCAGG (SEQ |
| | ID NO: 95) | ID NO: 96) |
| CSCR4 | AGGAGTTAGCCAAGATGTGACT | AGTCATTGGGGTAGAAGCGG (SEQ |
| | (SEQ ID NO: 97) | ID NO: 98) |
| SOX2 | CATGAAGGAGCACCCGGATT (SEQ | TTCATGTGCGCGTAACTGTC (SEQ |
| | ID NO: 99) | ID NO: 100) |
| ID3 | TGCCCACTTGACTTCACCAA (SEQ | GTTCACAGTCCTTCGCTCCT (SEQ |
| | ID NO: 101) | ID NO: 102) |
| FTL | GCTCCTTCTTGCCAACCAAC (SEQ | GCCCAGAGAGAGGTAGGTGT (SEQ |
| | ID NO: 103) | ID NO: 104) |
| TUBB1B | TGTTCACTGGTACGTGGGTG (SEQ | GCTGAAATTCTGGGAGCATGAC |
| | ID NO: 105) | (SEQ ID NO: 106) |

Discussion

Here we applied a technological strategy of combining iPSCs derived from a patient with WS, CRISPR/Cas9 correction of the disease-causing pathogenic gene variant, and our recent robust differentiation protocol to generate SC-β cells that were able to successfully and rapidly rescue mice with severe pre-existing diabetes. Correction of WFS1 drastically improved SC-β cell differentiation efficacy and function in the SC-β cells, which displayed robust first- and second-phase insulin secretion. scRNA-seq and direct assessment of the ER and mitochondria revealed reduction of stress in these organelles and a reduction in apoptotic markers after CRISPR/Cas9 correction.

Prior work with WS patient-derived iPSCs in the context of diabetes did not utilize gene therapy nor generate SC-β cells. Instead, this work focused on earlier stages of development by generating and studying pancreatic progenitors and polyhormonal endocrine cells that lack bone fide β cell features, such as high insulin per cell, dynamic function, or diabetes reversal in mice upon transplantation (12). More recent work with patient-derived iPSCs or nuclear transfer stem cells having other forms of diabetes has instead generated SC-β cells, but these cells are still missing important features of mature β cells, most noteworthy being continued inferior function (18-22). There is also limited data comparing differentiation of nuclear transfer stem cells (19) and iPSCs (15-18, 21, 22) to SC-β cells, so more studies comparing these two potential autologous cell sources is needed, particularly as the immunogenicity of these cells is unclear (8). In terms of cell therapy, only prevention, not reversal, of diabetes in mice using SC-β cells derived from patients with diabetes has been reported. This required long maturation times in vivo (12-19 wk) and has only been successful with limited mouse numbers, and often with the inability to return blood glucose to normoglycemia (18, 19, 22). Overgrowths and other abnormal structures have also been observed in some of these reports (19, 22). In contrast, we showed reversal of severe pre-existing diabetes in all mice with corrected WS SC-β cell transplantation, with no observed teratoma or cystic structure formation by use of our differentiation strategy. We did observe overgrowths in the WS4^{unedit} SC-β cell transplants that likely resulted from the detected proliferating off-target mesenchyme and progenitors.

Our study demonstrates that CRISPR/Cas9 correction of a diabetes-inducing gene variant allows for robust differentiation of autologous SC-β cells capable of reversing severe pre-existing diabetes. Application of our strategy for treating diabetes in mice may enable development of autologous cell replacement therapy in patients with diabetes. By using patient-derived cells that have been gene corrected to fix diabetic variants and produce robustly functional cells, the need for the patient to take immunosuppressant drugs could be avoided for many forms of diabetes (8). Alternative strategies for encapsulating cells for immune protection are currently being explored with allogeneic products but still require improvements for efficacy and safety (54, 55). Instead, autogenic SC-β cell therapy is possible and would benefit from combination with transplantation using macroporous scaffolds to better enable retrievability (56, 57) or other cells or materials to promote engraftment, survival, and function (58-62).

There are some important limitations to note in this study and in its implications. We primarily studied cells from one patient with WS. While these results are promising, differences in clinical presentation of WS is known, for example, with age of diabetes diagnosis ranging from 3 wk to 16 yr after birth (63). Further in-depth study with other patients is warranted to better represent heterogeneity in WS and to expand to other monogenetic forms of diabetes. A concern in iPSC culture (64) and in CRISPR/Cas9 (65) is the induction of off-target mutations, which likely necessitates careful karyotyping and genomic sequencing before translation to actual clinical use. In addition, while the SC-β cells generated in this study display many of the features of bone fide primary β cells, they are still not fully mature in terms of gene expression and function (23). Improvements in differentiation protocols to fully mature SC-β cells would help enable clinical translation of this technology to patients with WS.

Another major feature of our corrected WS SC-β cells is their ability to display robust dynamic function in vitro, with first- and second-phase insulin secretion. This feature is weak, absent, or not demonstrated in other diabetic SC-β cell strategies (18, 20-22), indicating that our observation of robust improvements in function with CRISPR/Cas9 would have been difficult with alternate approaches. Demonstrating dynamic function in patient SC-β cells is important for the study of diabetes, as dynamic function is commonly lost early on in the disease (66-69).

Human WS patient-derived β cells are not normally available for study due to the rarity of the disease and death of β cells during disease progression (70), and our ability to differentiate these cells from patient iPSCs facilitates in-depth modeling of this disease. Current common models of WS, including Wfs1 knockout mice and insulinoma lines, are of limited value in the study of WS due to species differences (21, 27-29, 31, 32). Of importance, Wfs1 knockout mice have only mild diabetes, in contrast with WS patients having insulin-dependent diabetes (31, 71). Understanding of human WS progression is lacking, making development of effective therapies difficult, and although the scope of this report is focused on one WS patient, we hope it enables more in-depth mechanistic study. Our human iPSC-derived model of WS validated the presence of elevated ER stress, activation of the unfolded protein response, and defects in insulin processing (39, 40). The proportion of endocrine subtype markers was affected in our model, with relatively lower expression of glucagon and C-peptide/insulin and higher somatostatin with the WFS1 pathogenic variant. Focused study of the influence of WFS1 and ER stress on differentiation could provide new insights. A major limitation of this study is the depth that we were capable of investigating molecular control and signaling of cellular stress, predominantly because of the purity differences of the SC-β cell population based on WFS1 pathogenic state and compared to human islets. The use of single-cell RNA sequencing enabled examination of gene expression differences, however sorting SC-β cells would be an ideal option for more robust study. Patient-derived SC-β cells can serve as a human patient-specific model of WS for further study of molecular events in p cell failure, drug screening, and a source of autologous cells for therapy (8).

Single-cell sequencing and in vitro assays demonstrated that WS4^{unedit} SC-β cells display ER stress. ER stress is found in other types of diabetes, including Type 1 and 2, neonatal diabetes, maturity onset diabetes of the young (MODY), and many other disorders (30-35). Future studies investigating WS and other forms of diabetes are now more rigorously possible via our platform for the discovery of new biology, therapeutic compounds, and replacement cells. WS, neonatal diabetes, and MODY are all monogenic forms of diabetes, and through gene therapy using CRISPR/Cas9, the pathogenic variant in specific genes causing diabetes in these patients can be corrected. Our approach, leveraging CRISPR/Cas9 and a robust differentiation protocol, allows for rigorous study of these forms of diabetes for disease modeling and drug screening. In the future, we suspect our advanced strategy combining patient iPSCs, gene editing, and differentiation to high functioning SC-β cells and other cell types will produce a viable, personalized cell source for cell therapy in patients with diabetes and other degenerative disorders (72).

Materials and Methods

Study Design

The objective of this study was to analyze and transplant insulin-producing β cells into mice with pre-existing diabetes to determine the translational potential of differentiated iPSCs from a patient with diabetes, specifically WS, after CRISPR/Cas9 correction of the pathogenic diabetes-causing variant. We used CRISPR/Cas9 on three iPSC lines derived from patients with WS to generate three corrected iPSC lines, all of which were differentiated to SC-β cells. We transplanted WS4^{corr} SC-β cells, WS4^{unedit} SC-β cells, and cadaveric human islet β cells into nine, six, and four STZ-treated diabetic mice, respectively. STZ-treated diabetic and nondiabetic mice without transplants served as transplantation controls. Mouse groups were assigned randomly, and the study was not blinded. Transplanted mice were monitored through blood glucose measurements and blood serum collection, then sacrificed for ex vivo analysis. Nephrectomy surgery was performed on WS4^{corr} transplanted mice to confirm transplanted SC-β cells were the source of glucose tolerance and nondiabetic blood glucose concentrations. For all in vitro analyses, at least 3 differentiations were used. Sample size was not determined with a power calculation, and specific sample cells are defined for each dataset. Data collection was stopped at pre-determined, arbitrary times. No data was excluded.

Further methods details are available in the Supplementary Materials.

hiPSC Line Generation

This work as performed in accordance with the ESCRO Committee at Washington University in St. Louis. WS9^{unedit} and WS13^{unedit} iPSC lines were previously published as Wolf-2010-9 and Wolf-2010-13, respectively (36). WS4^{unedit} iPSC line was generated from a previously described WS patient (WU.WOLF-04) (25) as previously described (36) by the Genetic Engineering and iPSC Center (GEiC) at Washington University in St. Louis from skin fibroblast with Sendai viral reprogramming (Life Technologies).

CRISPR/Cas9 Gene Correction

CRISPR/Cas9 gene correction of the WFS1 pathogenic variants in the WS4 and WS13 iPSC lines was performed at GEiC at Washington University in St. Louis. Guide RNAs were generated to target the WFS1 variants and validated using next generation deep sequencing analysis (NGS) (73). YH421.WFS1.sp14 and YS422.WFS1.sp11 was selected for homology directed repair using a single stranded DNA oligo (ssODN) as a template to target the point mutation in WFS1 on allele 2 for WS4 iPSCs and allele 1 for WS13 iPSCs, respectively. The designated allele to correct was determined based on the highest CRISPR/Cas9 gRNA specificity. Cells with successful CRISPR/Cas9 correction of WFS1 were termed WS4^{corr}, and cells in which WFS1 retained the point mutation were termed WS4^{unedit}, as confirmed by targeted NGS (73). The top 5 off-target sequences were also confirmed with NGS. gRNA sequences used are listed in TABLE 1.

Stem Cell-Derived β Cell Differentiation

Undifferentiated cells were seeded at 5.2-7.3×10⁵ cells/cm² and differentiated performed as outlined in tables S4-6 to generate SC-β cells (23). Cells were aggregated 7-9 d into stage 6 on an OrbiShaker (Benchmark) at 100 RPM for assessment. Islets were purchased from Prodo Labs and cultured in islet media (TABLE 6) for comparison 24 hr after shipment arrival. Bright field images were captured with a Leica DMi1.

Glucose-Stimulated Insulin Secretion

For static GSIS, approximately 30 stage 6 clusters were placed in transwells (Corning), equilibrated for 1 hr in 2 mM glucose KRB (TABLE 6), and challenged with sequential 1-hr 2 and 20 mM KRB treatments. For dynamic GSIS, clusters were loaded into a cell chamber, perfused with buffer at 100 μL/min, and equilibrated in 2 mM glucose KRB for 90 min. The samples were then challenged for 12 min at 2 mM, 24 min at 20 mM, and 12 min at 2 mM glucose KRB, collecting effluent every 2-4 min. Human insulin was quantified with ELISA and data normalized to cell counts from Vi-Cell XR or DNA quantification using Quant-iT Picogreen dsDNA assay kit (Invitrogen).

Mouse Transplants

Animal studies were performed in accordance with Washington University IACUC. Mice were randomly designated for STZ treatment and transplantation groups. Mouse number per group was selected to allow for statistical significance based on our prior studies (15, 17, 18). Surgical procedures and follow up studies were performed by unblinded individuals. Male 7 wk old NOD.Cg-Prkdc^{scid} II2rg^{tm1Wjl}/SzJ (NSG) mice were purchased from Jackson Laboratories, rendered diabetic with injection of 45 mg/kg STZ (R&D) for 5 d, with diabetes confirmed after 8 d. Anaesthetized mice were injected with 5×10⁶ WS4^{unedit} stage 6 cells, 5×10⁶ WS4^{corr} stage 6 cells, 5×10⁶ (4000 IEQ) islet cells, or saline under the kidney capsule. Islet transplantation was performed in a separate cohort. Animals were monitored up to 6 months. Blood glucose was measured with a Contour Blood Glucose Monitoring System (Bayer). Glucose tolerance and in vivo GSIS assays were performed by fasting mice for 4 hr and injecting with 2 g/kg glucose. Serum hormones were quantified using Human Ultrasensitive Insulin ELISA kit (ALPCO Diagnostics), Mouse C-peptide ELISA (ALPCO Diagnostics), and human proinsulin ELISA (Mercodia). 12-wk after transplantation, live nephrectomy was performed on 2 anaesthetized transplanted mice.

Statistical Analysis

Statistical analysis was calculated with GraphPad Prism. Data was tested for normal (Gaussian) distribution using Shapiro-Wilk normality test. One- and two-sided unpaired and paired t-tests and one- and two-way ANOVA with Tukey's or Dunnett's tests were used for data sets with a normal distribution. Otherwise, Mann-Whitney two-tailed nonparametric tests were used. Statistical tests are specified in figure legends. p<0.05 was considered statistically significant. Data is shown as mean±s.e.m unless otherwise noted. The sample size, n, indicates the total number of biological replicates.

hiPSC Line Culture

Undifferentiated hiPSCs were cultured with mTeSR1 (StemCell Technologies) in an incubator with 5% CO₂ at 37° C. Cells were passaged every 3-4 days by single cell dispersion using TrypLE (Life Technologies). Viable cells were counted with a Vi-Cell XR (Beckman Coulter) and seeded onto DMEM-diluted (Gibco) Matrigel-coated (Fisher) culture flasks at 1.1×10⁵ cells/cm² in mTESR1 with 10 μM Y27532 (Abcam).

Immunostaining

Flow cytometry staining and analysis was performed by dispersing and fixing single cells in 4% paraformaldehyde (PFA) (Electron Microscopy Science) 30 min at RT, blocking, and staining with primary antibodies diluted in immunostaining solution (TABLE 6) overnight at 4° C., staining with second antibodies for 2 hr at 4° C., and analyzed on the LSRII and BD LSR Fortessa X-20 (BD Biosciences). FlowJo generated dot plots and percentages. Processing and staining sections was performed by fixing clusters or transplanted kidneys in 4% PFA overnight at 4° C., placed in Histogel (Thermo Scientific), and processed by the Division of Comparative Medicine (DCM) Research Animal Diagnostic Laboratory Core at Washington University. Paraffin was removed with histoclear (Thermo Scientific), samples rehydrated, antigens were retrieved with 0.05 M EDTA (Ambion) in a pressure cooker (Proteogenix; 2100 Retriever), immunostaining performed as above, and samples mounted with DAPI Fluoromount-G (SouthernBiotech). Processing and staining plated cells on black tissue culture 96 well plates (Corning) was performed by fixing with 4% PFA 30 min at RT, immunostaining as above, and staining for DAPI. Immunostained cells were imaged with a Leica DMI4000 fluorescence or Nikon A1Rsi confocal microscope. Antibody details can be found in TABLE 7.

Western Blots

Detection of phosphorylated and total eIF2α as performed by lysing cells in 1% Triton buffer (20 mM HEPES, pH 7.5, 150 mM NaCl, 1% Triton X-100, 10% glycerol (Sigma), 1 mM EDTA, 10 mM tetrasodium pyrophosphate (Sigma), 17.5 mM β-glycerophosphate (Sigma), PhosSTOP (Roche), and complete protease inhibitor cocktail (Roche). Detection of all other proteins was done by lysing cells in M-PER Mammalian Protein Extraction Reagent (Thermo Scientific) supplemented with cOmplete protease inhibitor cocktail. After 30 min incubation on ice, samples were centrifuged, supernatants collected, and protein-equivalent samples were loaded onto 4-20% Mini-PROTEAN TGX Precast Protein Gels (Bio-Rad) and transferred to an Immobilon-P PVDF Membrane (Millipore). The membrane was treated with 5% skimmed milk for 1 hr at RT and probed with the primary antibodies. The antibodies were detected using HRP-linked secondary antibodies. The density of each band was quantified using Image Lab software (Bio-Rad). Antibodies used are listed in TABLE 7.

Single-Cell RNA Sequencing Analysis

Library preparation and sequencing was performed at the Washington University in St. Louis Genome Technology Access Center using the Chromium Single Cell 3′ Library and Gel Bead Kit v2 (10× Genomics) and the HiSeq2500 System (Illumina) at 26×98 bp. Cells containing high mitochondrial percentages were filtered out, and only cells containing 50-7500 genes were used for analysis. Seurat v2.0 was used to perform unsupervised clustering (42).

Seurat v2.0 analysis was used to perform unsupervised clustering (42). We used global-scaling normalization and removed unwanted variation calculated by scaled z-score dispersion. We inputted the variable genes into principle component analysis (PCA) to perform linear dimensional reduction and identify statistically significant principal components (PCs). JackStraw recalculated the PCA with a null distribution of gene scores to reduce noise. The new PCs were selected based on genes with strong enrichment of low p-values. Cells with similar gene expression patterns are located near each other based on PCA using the FindClusters function. We used a resolution of 0.4 and 10 dimensions for WS4^{unedit} and WS4^{corr} cells to present clusters on tSNE plots with single points representing individual cells. The differential gene expression pattern, that distinguished each cluster from all other cells, was found with the FindAllMarkers function. The top 50 genes for each cluster was used to define the cluster cell type. The genes were compared to current single cell pancreas transcriptomes to define cell types (43, 74). Feature plots using a tSNE plot were used to visualize gene expression across different populations.

Differential expression between WS4^{unedit} and WS4^{corr} cells: Seurat v2.0 was used to combined the two objects (WS4^{unedit} and WS4^{corr} SC-β cells) and perform canonical correlation analyses (CCA) (Function: RunCCA) to remove any sources of variation between the two objects (42). Next, the CCA subspaces were aligned (Function: AlignSubspace) with 50 dimensions to create dimensional reduction in order to perform clustering. A single integrated analysis was then performed on the combined object. Clusters were defined using FindClusters (resolution=0.6, 10 dimensions) and a tSNE plot was generated using RunTSNE. The gene markers that were upregulated in each cluster, regardless of WS4^{unedit} or WS4^{corr} SC-β cells, were defined based on p-values using the function, FindConservedMarkers. The top 50 genes for each cluster were used to define the cluster cell type. The genes were compared to current single cell pancreas transcriptomes to define the cell types (43, 74). We were able to visualize differences in gene expression with violin plots (VInPlot) between, specifically the β cells, in the WS4^{unedit} and WS4^{corr} objects for β cell markers, pancreatic markers, and ER stress genes. As the β cell population did not contain off target markers, we used violin plots to compare the off-target marker gene expression between entire WS4^{unedit} and WS4^{corr} objects. We performed statistics of the violin plots evaluating ER stress, β, and islet cell markers using the FindAllMarkers function (log fc.threshold=0) which provided unadjusted and adjusted p values based on percentage of cells where gene was detected (pct.1 for WS4^{corr}; pct.2 for WS4^{unedit}). In addition, the log fold-change of average expression between WS4^{corr} and WS4^{unedit} (avg_log FC) was calculated. The positive and negative values indicate the gene is more highly expressed in WS4^{corr} or WS4^{unedit}, respectively.

Real Time PCR

RNA was extracted with the RNeasy Mini Kit (Qiagen) with DNase treatment (Qiagen), cDNA was synthesized with the High Capacity cDNA Reverse Transcriptase Kit (Applied Biosystems using a thermocycler (Applied Biosystems), real-time PCR reactions were generated with PowerUp SYBR Green Master Mix (Applied Biosystems) using a StepOnePlus (Applied Biosystems), and data analyzed using ΔΔCt methodology. TBP and GUSB were used as normalization genes. Primer sequences used can be found in TABLE 8.

Electron Microscopy

Cells were fixed for 2 hr at RT with 2.5% glutaraldehyde, 1.25% PFA, and 0.03% picric acid in 0.1 M sodium cacodylate buffer at pH 7.4 and shipped to Harvard Medical School Electron Microscopy Core Facility for processing and sectioning. The sectioned samples were imaged on the JEM-1400+Transmission Electron Microscope (JEOL), running at 120 kV. Size of endoplasmic reticulum and mitochondria organelles were quantified with ImageJ. Some of the images show overlapping views.

Insulin and Proinsulin Content

Clusters were immersed in 1.5% HCl and 70% ethanol for 72 hr at −20° C., with periodic vortex, centrifuged for 15 min at 2100 RCF, supernatant collected, pH neutralized with equal volume of 1 M TRIS (pH 7.5), and hormones quantified with ELISA, with cell count normalization (Vi-Cell XR).

Mitochondrial Respiration Studies

Cells were seeded on Seahorse microplates (Agilent) overnight, media replaced with RPMI 1640 (Sigma) at 7.4 pH in each well, and the cells placed in a Seahorse XFe24 extracellular flux analyzer (Agilent). After 4 baseline measurements, cells were treated with sequential injections of 3 μM oligomycin (Calbiochem), 0.25 μM carbonyl cyande-4-(trifluoromethoxy) phenylhydrazone (FCCP) (Sigma) and 1 μM rotenone (Calbiochem) and 2 μM antimycin A (Sigma).

ER Stress Studies

Cells were incubated for 48 hr with 10 μM Thapsigargin (EMD Chemicals), 30 mM glucose, or a cytokine mix including 500 ng/mL IFNγ (R&D Systems), 500 ng/mL TNFα (R&D Systems), and 100 ng/mL IL-1R (R&D Systems).

### REFERENCES

1. Karagiannis P, Takahashi K, Saito M, Yoshida Y, Okita K, Watanabe A, Inoue H, Yamashita J K, Todani M, Nakagawa M. J. P. r., Induced pluripotent stem cells and their use in human models of disease and development. 99, 79-114 (2018).
2. Takahashi K, Tanabe K, Ohnuki M, Narita M, Ichisaka T, Tomoda K, Yamanaka S. J. c., Induction of pluripotent stem cells from adult human fibroblasts by defined factors. 131, 861-872 (2007).
3. Atkinson M A, Eisenbarth G S, Michels A W, Type 1 diabetes. Lancet 383, 69-82 (2014).
4. Nathan D M, Long-term complications of diabetes mellitus. N Engl J Med 328, 1676-1685 (1993).
5. Shapiro A M, Lakey J R, Ryan E A, Korbutt G S, Toth E, Warnock G L, Kneteman N M, Rajotte R V, Islet transplantation in seven patients with type 1 diabetes mellitus using a glucocorticoid-free immunosuppressive regimen. N Engl J Med 343, 230-238 (2000).
6. Shapiro A M, Ricordi C, Hering B J, Auchincloss H, Lindblad R, Robertson R P, Secchi A, Brendel M D, Berney T, Brennan D C, Cagliero E, Alejandro R, Ryan E A, DiMercurio B, Morel P, Polonsky K S, Reems J A, Bretzel R G, Bertuzzi F, Froud T, Kandaswamy R, Sutherland D E, Eisenbarth G, Segal M, Preiksaitis J, Korbutt G S, Barton F B, Viviano L, Seyfert-Margolis V, Bluestone J, Lakey J R, International trial of the Edmonton protocol for islet transplantation.

N Engl J Med 355, 1318-1330 (2006).
7. Scharp D W, Lacy P E, Santiago J V, McCullough C S, Weide L G, Falqui L, Marchetti P, Gingerich R L, Jaffe A S, Cryer P E, et al., Insulin independence after islet transplantation into type I diabetic patient. Diabetes 39, 515-518 (1990).
8. Millman J R, Pagliuca F W, Autologous Pluripotent Stem Cell-Derived beta-Like Cells for Diabetes Cellular Therapy. Diabetes 66, 1111-1120 (2017).
9. Odorico J, Markmann J, Melton D, Greenstein J, Hwa A, Nostro C, Rezania A, Oberholzer J, Pipeleers D, Yang L, Cowan C, Huangfu D, Egli D, Ben-David U, Vallier L, Grey S T, Tang Q, Roep B, Ricordi C, Naji A, Orlando G, Anderson D G, Poznansky M, Ludwig B, Tomei A, Greiner D L, Graham M, Carpenter M, Migliaccio G, D'Amour K, Hering B, Piemonti L, Berney T, Rickels M, Kay T, Adams A, Report of the Key Opinion Leaders Meeting on Stem Cell-derived Beta Cells. Transplantation 102, 1223-1229 (2018).
10. Maehr R, Chen S, Snitow M, Ludwig T, Yagasaki L, Goland R, Leibel R L, Melton D A, Generation of pluripotent stem cells from patients with type 1 diabetes. Proceedings of the National Academy of Sciences of the United States of America 106, 15768-15773 (2009).
11. Thatava T, Kudva Y C, Edukulla R, Squillace K, De Lamo J G, Khan Y K, Sakuma T, Ohmine S, Terzic A, Ikeda Y, Intrapatient variations in type 1 diabetes-specific iPS cell differentiation into insulin-producing cells. Mol Ther 21, 228-239(2013).
12. Shang L, Hua H, Foo K, Martinez H, Watanabe K, Zimmer M, Kahler D J, Freeby M, Chung W, LeDuc C, Goland R, Leibel R L, Egli D, beta-cell dysfunction due to increased E R stress in a stem cell model of Wolfram syndrome. Diabetes 63, 923-933 (2014).
13. Hua H, Shang L, Martinez H, Freeby M, Gallagher M P, Ludwig T, Deng L, Greenberg E, Leduc C, Chung W K, Goland R, Leibel R L, Egli D, iPSC-derived beta cells model diabetes due to glucokinase deficiency. The Journal of clinical investigation 123, 3146-3153 (2013).
14. Yamada M, Johannesson B, Sagi I, Burnett L C, Kort D H, Prosser R W, Paull D, Nestor M W, Freeby M, Greenberg E, Goland R S, Leibel R L, Solomon S L, Benvenisty N, Sauer M V, Egli D, Human oocytes reprogram adult somatic nuclei of a type 1 diabetic to diploid pluripotent stem cells. Nature 510, 533-536 (2014).
15. Pagliuca F W, Millman J R, Gurtler M, Segel M, Van Dervort A, Ryu J H, Peterson Q P, Greiner D, Melton D A, Generation of functional human pancreatic beta cells in vitro. Cell 159, 428-439 (2014).
16. Rezania A, Bruin J E, Arora P, Rubin A, Batushansky I, Asadi A, O'Dwyer S, Quiskamp N, Mojibian M, Albrecht T, Yang Y H, Johnson J D, Kieffer T J, Reversal of diabetes with insulin-producing cells derived in vitro from human pluripotent stem cells. Nature biotechnology 32, 1121-1133 (2014).
17. Velazco-Cruz L, Song J, Maxwell K G, Goedegebuure M M, Augsornworawat P, Hogrebe N J, Millman J R, Acquisition of Dynamic Function in Human Stem Cell-Derived beta Cells. Stem cell reports 12, 351-365 (2019).
18. Millman J R, Xie C, Van Dervort A, Gurtler M, Pagliuca F W, Melton D A, Generation of stem cell-derived beta-cells from patients with type 1 diabetes. Nat Commun 7, 11463 (2016).
19. Sui L, Danzl N, Campbell S R, Viola R, Williams D, Xing Y, Wang Y, Phillips N, Poffenberger G, Johannesson B, Oberholzer J, Powers A C, Leibel R L, Chen X, Sykes M, Egli D, beta-Cell Replacement in Mice Using Human Type 1 Diabetes Nuclear Transfer Embryonic Stem Cells. Diabetes 67, 26-35 (2018).
20. Wang X, Sterr M, Ansarullah, Burtscher I, Böttcher A, Beckenbauer J, Siehler J, Meitinger T, Hsring H-U, Staiger H, Cernilogar F M, Schotta G, Irmler M, Beckers J, Wright CVE, Bakhti M, Lickert H, Point mutations in the PDX1 transactivation domain impair human R-cell development and function. Molecular metabolism, (2019).
21. Balboa D, Saarimaki-Vire J, Borshagovski D, Survila M, Lindholm P, Galli E, Eurola S, Ustinov J, Grym H, Huopio H, Partanen J, Wartiovaara K, Otonkoski T, Insulin mutations impair beta-cell development in a patient-derived iPSC model of neonatal diabetes. eLife 7, (2018).
22. Ma S, Viola R, Sui L, Cherubini V, Barbetti F, Egli D, beta Cell Replacement after Gene Editing of a Neonatal Diabetes-Causing Mutation at the Insulin Locus. Stem cell reports 11, 1407-1415 (2018).
23. Hogrebe N J, Augsornworawat P, Maxwell K G, Velazco-Cruz L, Millman J R, Targeting the cytoskeleton to direct pancreatic differentiation of human pluripotent stem cells. Nature biotechnology, (2020).
24. Hsu P D, Lander E S, Zhang F, Development and applications of CRISPR-Cas9 for genome engineering. Cell 157, 1262-1278 (2014).
25. Marshall B A, Permutt M A, Paciorkowski A R, Hoekel J, Karzon R, Wasson J, Viehover A, White N H, Shimony J S, Manwaring L, Austin P, Hullar T E, Hershey T, W. U. W. S. Grp, Phenotypic characteristics of early Wolfram syndrome. Orphanet J Rare Dis 8, (2013).
26. Urano F, Wolfram Syndrome: Diagnosis, Management, and Treatment. Current diabetes reports 16, 6 (2016).
27. Fonseca S G, Fukuma M, Lipson K L, Nguyen L X, Allen J R, Oka Y, Urano F, WFS1 is a novel component of the unfolded protein response and maintains homeostasis of the endoplasmic reticulum in pancreatic beta-cells. The Journal of biological chemistry 280, 39609-39615 (2005).
28. Fonseca S G, Ishigaki S, Oslowski C M, Lu S, Lipson K L, Ghosh R, Hayashi E, Ishihara H, Oka Y, Permutt M A, Urano F, Wolfram syndrome 1 gene negatively regulates E R stress signaling in rodent and human cells. The Journal of clinical investigation 120, 744-755 (2010).
29. Yamada T, Ishihara H, Tamura A, Takahashi R, Yamaguchi S, Takei D, Tokita A, Satake C, Tashiro F, Katagiri H, Aburatani H, Miyazaki J, Oka Y, WFS1-deficiency increases endoplasmic reticulum stress, impairs cell cycle progression and triggers the apoptotic pathway specifically in pancreatic beta-cells. Hum Mol Genet 15, 1600-1609 (2006).
30. Araki E, Oyadomari S, Mori M, Impact of endoplasmic reticulum stress pathway on pancreatic beta-cells and diabetes mellitus. Exp Biol Med (Maywood) 228, 1213-1217 (2003).
31. Riggs A C, Bernal-Mizrachi E, Ohsugi M, Wasson J, Fatrai S, Welling C, Murray J, Schmidt R E, Herrera P L, Permutt M A, Mice conditionally lacking the Wolfram gene in pancreatic islet beta cells exhibit diabetes as a result of enhanced endoplasmic reticulum stress and apoptosis. Diabetologia 48, 2313-2321 (2005).
32. Fonseca S G, Gromada J, Urano F, Endoplasmic reticulum stress and pancreatic beta-cell death. Trends in endocrinology and metabolism: TEM 22, 266-274 (2011).
33. Harding H P, Ron D, Endoplasmic reticulum stress and the development of diabetes: a review. Diabetes 51 Suppl 3, S455-461 (2002).
34. Ozcan U, Cao Q, Yilmaz E, Lee A H, Iwakoshi N N, Ozdelen E, Tuncman G, Gorgun C, Glimcher L H, Hotamisligil G S, Endoplasmic reticulum stress links obesity, insulin action, and type 2 diabetes. Science (New York, N.Y 306, 457-461 (2004).
35. Zhong J, Rao X, Xu J F, Yang P, Wang C Y, The role of endoplasmic reticulum stress in autoimmune-mediated beta-cell destruction in type 1 diabetes. Exp Diabetes Res 2012, 238980 (2012).
36. Lu S, Kanekura K, Hara T, Mahadevan J, Spears L D, Oslowski C M, Martinez R, Yamazaki-Inoue M, Toyoda M, Neilson A, A calcium-dependent protease as a potential therapeutic target for Wolfram syndrome. Proceedings of the National Academy of Sciences 111, E5292-E5301 (2014).
37. Lombardi A A, Gibb A A, Arif E, Kolmetzky D W, Tomar D, Luongo T S, Jadiya P, Murray E K, Lorkiewicz P K, Hajnoczky G, Murphy E, Arany Z P, Kelly D P, Margulies K B, Hill B G, Elrod J W, Mitochondrial calcium exchange links metabolism with the epigenome to control cellular differentiation. Nat Commun 10, 4509 (2019).
38. Mody N, Parhami F, Sarafian T A, Demer L L, Oxidative stress modulates osteoblastic differentiation of vascular and bone cells. Free Radic Biol Med 31, 509-519 (2001).
39. Hofmann S, Bauer M F, Wolfram syndrome-associated mutations lead to instability and proteasomal degradation of wolframin. FEBS letters 580, 4000-4004 (2006).
40. de Heredia M L, Cleries R, Nunes V, Genotypic classification of patients with Wolfram syndrome: insights into the natural history of the disease and correlation with phenotype. Genet Med 15, 497-506 (2013).
41. Macosko E Z, Basu A, Satija R, Nemesh J, Shekhar K, Goldman M, Tirosh I, Bialas A R, Kamitaki N, Martersteck EMJC, Highly parallel genome-wide expression profiling of individual cells using nanoliter droplets. 161, 1202-1214 (2015).
42. Butler A, Hoffman P, Smibert P, Papalexi E, Satija R. J. N. b., Integrating single-cell transcriptomic data across different conditions, technologies, and species. 36, 411 (2018).
43. Muraro M J, Dharmadhikari G, Gran D, Groen N, Dielen T, Jansen E, van Gurp L, Engelse M A, Carlotti F, de Koning E. J. J. C. s., A single-cell transcriptome atlas of the human pancreas. 3, 385-394. e383 (2016).
44. Arda H E, Li L, Tsai J, Torre E A, Rosli Y, Peiris H, Spitale R C, Dai C, Gu X, Qu K, Wang P, Wang J, Grompe M, Scharfmann R, Snyder M S, Bottino R, Powers A C, Chang H Y, Kim S K, Age-Dependent Pancreatic Gene Regulation Reveals Mechanisms Governing Human beta Cell Function. Cell metabolism 23, 909-920 (2016).
45. Segerstolpe A, Palasantza A, Eliasson P, Andersson E M, Andreasson A C, Sun X, Picelli S, Sabirsh A, Clausen M, Bjursell M K, Smith D M, Kasper M, Ammala C, Sandberg R, Single-Cell Transcriptome Profiling of Human Pancreatic Islets in Health and Type 2 Diabetes. Cell metabolism 24, 593-607 (2016).
46. Petersen MBK, Azad A, Ingvorsen C, Hess K, Hansson M, Grapin-Botton A, Honore C, Single-Cell Gene Expression Analysis of a Human ESC Model of Pancreatic Endocrine Development Reveals Different Paths to beta-Cell Differentiation. Stem cell reports 9, 1246-1261 (2017).
47. Arunagiri A, Haataja L, Cunningham C N, Shrestha N, Tsai B, Qi L, Liu M, Arvan P, Misfolded proinsulin in the endoplasmic reticulum during development of beta cell failure in diabetes. Ann N Y Acad Sci 1418, 5-19 (2018).
48. Maechler P, Wollheim C B, Mitochondrial function in normal and diabetic beta-cells. Nature 414, 807-812 (2001).
49. Rötig A, Cormier V, Chatelain P, Francois R, Saudubray J, Rustin P, Munnich A, Deletion of mitochondrial DNA in a case of early-onset diabetes mellitus, optic atrophy, and deafness (Wolfram syndrome, MIM 222300). The Journal of clinical investigation 91, 1095-1098 (1993).
50. Cagalinec M, Liiv M, Hodurova Z, Hickey M A, Vaarmann A, Mandel M, Zeb A, Choubey V, Kuum M, Safiulina D, Role of mitochondrial dynamics in neuronal development: mechanism for wolfram syndrome. PLoS biology 14, e1002511 (2016).
51. Nair G G, Liu J S, Russ H A, Tran S, Saxton M S, Chen R, Juang C, Li M L, Nguyen V Q, Giacometti S, Puri S, Xing Y, Wang Y, Szot G L, Oberholzer J, Bhushan A, Hebrok M, Recapitulating endocrine cell clustering in culture promotes maturation of human stem-cell-derived beta cells. Nature cell biology 21, 263-274 (2019).
52. Millman J R, Doggett T, Thebeau C, Zhang S, Semenkovich C F, Rajagopal R, Measurement of Energy Metabolism in Explanted Retinal Tissue Using Extracellular Flux Analysis. J Vis Exp, (2019).
53. Cardenas-Diaz F L, Osorio-Quintero C, Diaz-Miranda M A, Kishore S, Leavens K, Jobaliya C, Stanescu D, Ortiz-Gonzalez X, Yoon C, Chen C S, Haliyur R, Brissova M, Powers A C, French D L, Gadue P, Modeling Monogenic Diabetes using Human ESCs Reveals Developmental and Metabolic Deficiencies Caused by Mutations in HNF1A. Cell stem cell 25, 273-289 e275 (2019).
54. Tomei A A, Villa C, Ricordi C, Development of an encapsulated stem cell-based therapy for diabetes. Expert Opin Biol Ther 15, 1321-1336 (2015).
55. Scharp D W, Marchetti P, Encapsulated islets for diabetes therapy: history, current progress, and critical issues requiring solution. Adv Drug Deliv Rev 67-68, 35-73 (2014).
56. Pedraza E, Brady A C, Fraker C A, Molano R D, Sukert S, Berman D M, Kenyon N S, Pileggi A, Ricordi C, Stabler C L, Macroporous three-dimensional PDMS scaffolds for extrahepatic islet transplantation. Cell Transplant 22, 1123-1135 (2013).
57. Song J, Millman J R, Economic 3D-printing approach for transplantation of human stem cell-derived beta-like cells. Biofabrication 9, 015002 (2016).
58. Najjar M, Manzoli V, Abreu M, Villa C, Martino M M, Molano R D, Torrente Y, Pileggi A, Inverardi L, Ricordi C, Hubbell J A, Tomei A A, Fibrin gels engineered with pro-angiogenic growth factors promote engraftment of pancreatic islets in extrahepatic sites in mice. Biotechnology and bioengineering 112, 1916-1926 (2015).
59. Sackett S D, Tremmel D M, Ma F, Feeney A K, Maguire R M, Brown M E, Zhou Y, Li X, O'Brien C, Li L, Burlingham W J, Odorico J S, Extracellular matrix scaffold and hydrogel derived from decellularized and delipidized human pancreas. Sci Rep 8, 10452 (2018).
60. Tremmel D M, Odorico J S, Rebuilding a better home for transplanted islets. Organogenesis 14, 163-168 (2018).
61. Jiang K, Chaimov D, Patel S N, Liang J P, Wiggins S C, Samojlik M M, Rubiano A, Simmons C S, Stabler C L, 3-D physiomimetic extracellular matrix hydrogels provide a supportive microenvironment for rodent and human islet culture. Biomaterials 198, 37-48 (2019).
62. Augsornworawat P, Velazco-Cruz L, Song J, Millman J R, A hydrogel platform for in vitro three dimensional assembly of human stem cell-derived islet cells and endothelial cells. Acta Biomater, (2019).
63. Barrett T G, Bundey S E, Macleod A F, Neurodegeneration and diabetes: UK nationwide study of Wolfram (DIDMOAD) syndrome. Lancet 346, 1458-1463 (1995).
64. Gore A, Li Z, Fung H L, Young J E, Agarwal S, Antosiewicz-Bourget J, Canto I, Giorgetti A, Israel M A, Kiskinis E, Lee J H, Loh Y H, Manos P D, Montserrat N, Panopoulos A D, Ruiz S, Wilbert M L, Yu J, Kirkness E F, Izpisua Belmonte J C, Rossi D J, Thomson J A, Eggan K, Daley G Q, Goldstein L S, Zhang K, Somatic coding mutations in human induced pluripotent stem cells. Nature 471, 63-67 (2011).
65. Fu Y, Foden J A, Khayter C, Maeder M L, Reyon D, Joung J K, Sander J D, High-frequency off-target mutagenesis induced by CRISPR-Cas nucleases in human cells. Nature biotechnology 31, 822-826 (2013).
66. Caumo A, Luzi L, First-phase insulin secretion: does it exist in real life? Considerations on shape and function. Am J Physiol Endocrinol Metab 287, E371-385 (2004).
67. Del Prato S, Tiengo A, The importance of first-phase insulin secretion: implications for the therapy of type 2 diabetes mellitus. Diabetes/metabolism research and reviews 17, 164-174 (2001).
68. Seino S, Shibasaki T, Minami K, Dynamics of insulin secretion and the clinical implications for obesity and diabetes. The Journal of clinical investigation 121, 2118-2125 (2011).
69. Zhang C Y, Baffy G, Perret P, Krauss S, Peroni O, Grujic D, Hagen T, Vidal-Puig A J, Boss O, Kim Y B, Zheng X X, Wheeler M B, Shulman G I, Chan C B, Lowell B B, Uncoupling protein-2 negatively regulates insulin secretion and is a major link between obesity, beta cell dysfunction, and type 2 diabetes. Cell 105, 745-755 (2001).
70. Hilson J B, Merchant S N, Adams J C, Joseph J T, Wolfram syndrome: a clinicopathologic correlation. Acta Neuropathol 118, 415-428 (2009).
71. Ishihara H, Takeda S, Tamura A, Takahashi R, Yamaguchi S, Takei D, Yamada T, Inoue H, Soga H, Katagiri H, Tanizawa Y, Oka Y, Disruption of the WFS1 gene in mice causes progressive beta-cell loss and impaired stimulus-secretion coupling in insulin secretion. Hum Mol Genet 13, 1159-1170 (2004).
72. Abreu D, Urano F, Current Landscape of Treatments for Wolfram Syndrome. Trends Pharmacol Sci, (2019).
73. Bell C C, Magor G W, Gillinder K R, Perkins A C, A high-throughput screening strategy for detecting CRISPR-Cas9 induced mutations using next-generation sequencing. BMC Genomics 15, 1002 (2014).
74. Baron M, Veres A, Wolock S L, Faust A L, Gaujoux R, Vetere A, Ryu J H, Wagner B K, Shen-Orr S S, Klein A M, Melton D A, Yanai I, A Single-Cell Transcriptomic Map of the Human and Mouse Pancreas Reveals Inter- and Intra-cell Population Structure. Cell Syst, (2016).

### Example 2: Improvements in Differentiation into Sc-B Cells

The following example describes the compositions and methods for enhancing the directed differentiation protocol and for making and studying SC-β cells. Methods and compositions described include: re-aggregating stage 6 cells in spinner flasks; cryopreservation of S C-beta cells; microenvironmental cues to enhance S C-beta cell differentiation and maturation; assays to evaluate the effect of chemical and/or genetic stress on S C-islets; cell hashing stress beta cells; using mCherry/INS reporter cell lines to study beta cell health; refining stage 6 enriched serum-free media for S C-beta cells; modulating stage 2 duration effects on pancreatic differentiation; compounds to improve S C-beta cells; and ECM proteins and stiffness influence S C-beta cell function and maturation.

(I) Re-Aggregating Stage 6 Cells in Spinner Flasks

Stage 6 cells can be re-aggregated into clusters after single cell dispersing and seeding into biott spinner flasks at a concentration of 1 million cells per ml of stage 6 media and at 55 RPM.

Being able to re-aggregate stage 6 SC-β cells in spinner flasks (rather than with the 6-well plate aggregation method we currently use) would facilitate large scale up of this process.

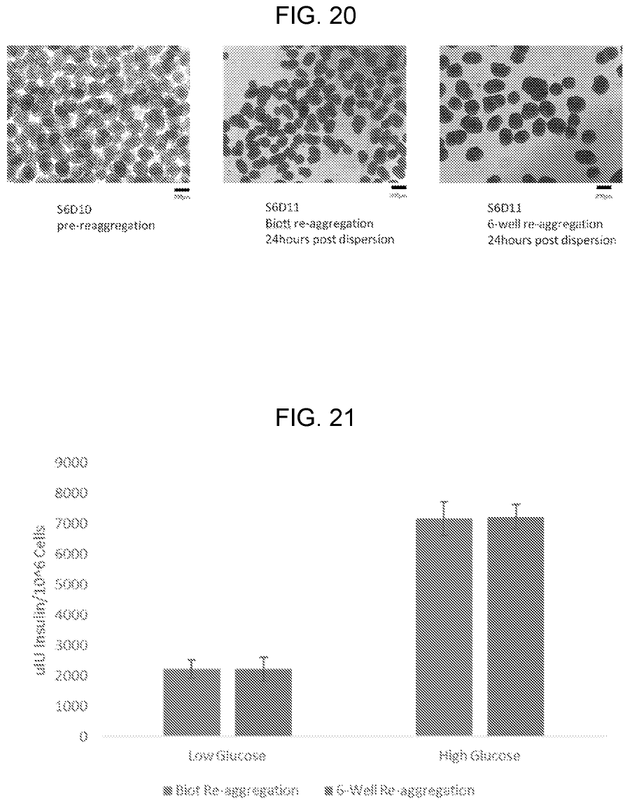

SC-β cells are capable of re-aggregating into clusters within spinner flasks after dispersion from clusters (see e.g., FIG. 20). SC-β cells re-aggregate in spinner flasks without loss of function (see e.g., FIG. 21).

(II) Cryopreservation of SC-β Cells

Stage 6 SC-β cells can be single cell dispersed, cryopreserved, and thawed, and retain function and marker expression.

The methods described here allow for the cryopreservation of SC-β cells, allowing for shipping of SC-β cells as well as quality control and assurance of an SC-β cell product from large batches.

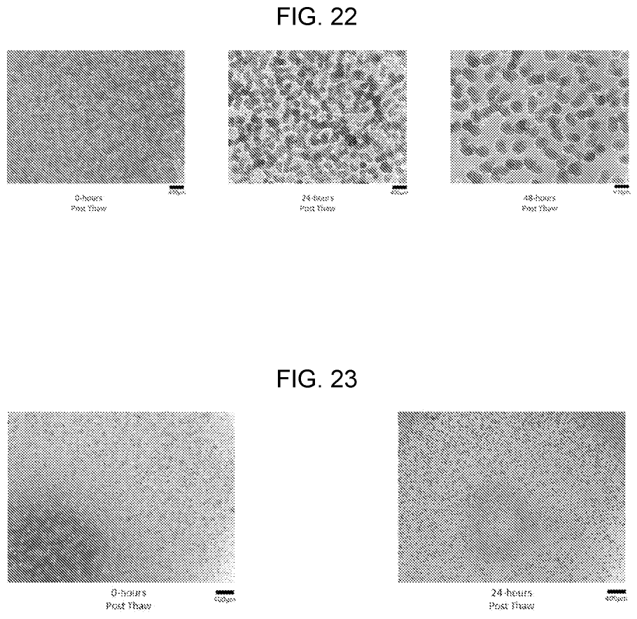

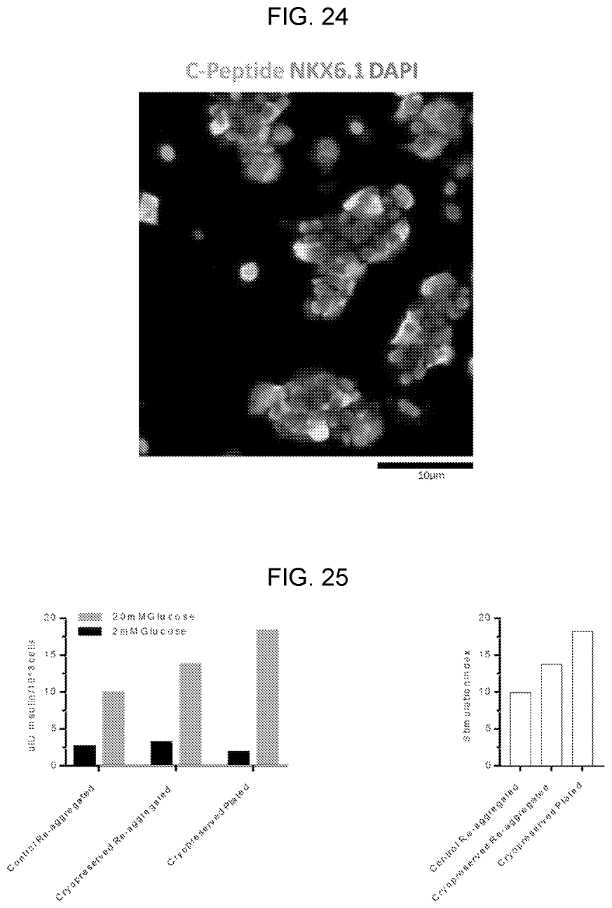

Thawed cryopreserved SC-β cells re-aggregate after thawing when cultured in suspension (see e.g., FIG. 22). Thawed cryopreserved SC-β cells adhere when cultured on Matrigel coated plastic (see e.g., FIG. 23). Thawed cryopreserved SC-β cells maintain marker expression (FIG. 24). Thawed cryopreserved SC-β cells remain function showing glucose stimulated insulin secretion (FIG. 25).

(III) Microenvironmental Cues to Enhance SC-β Cell Differentiation and Function

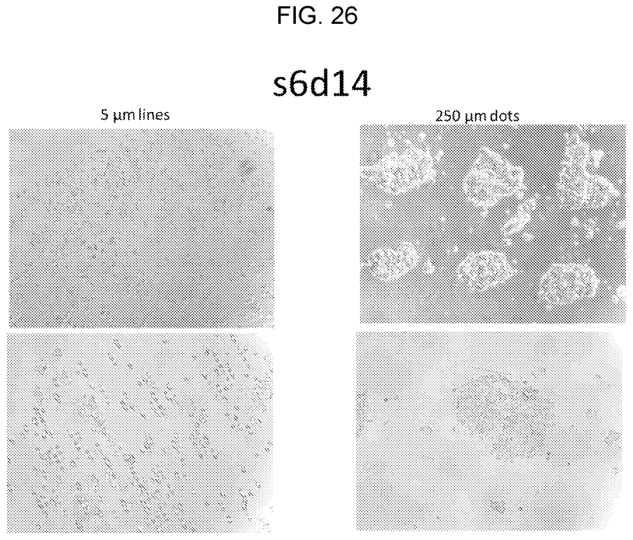

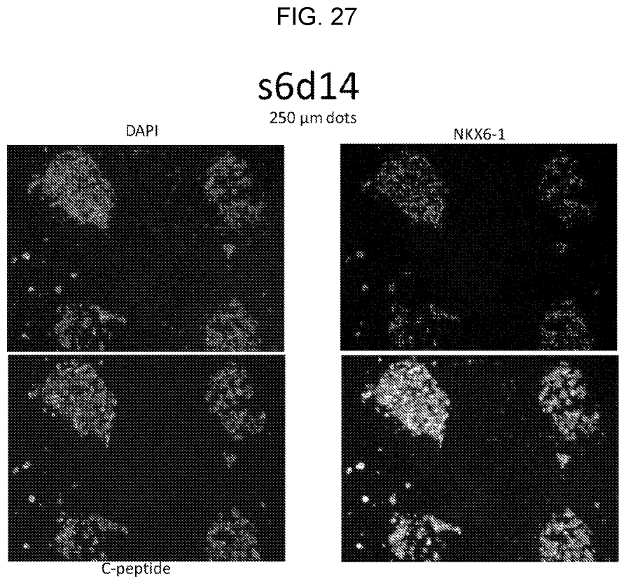

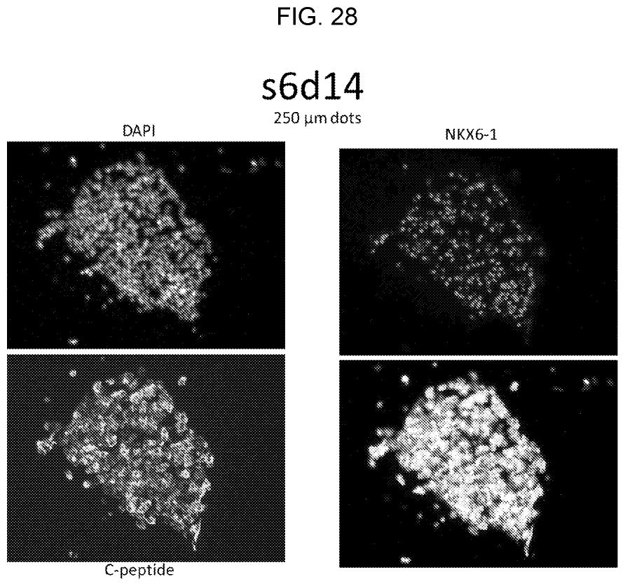

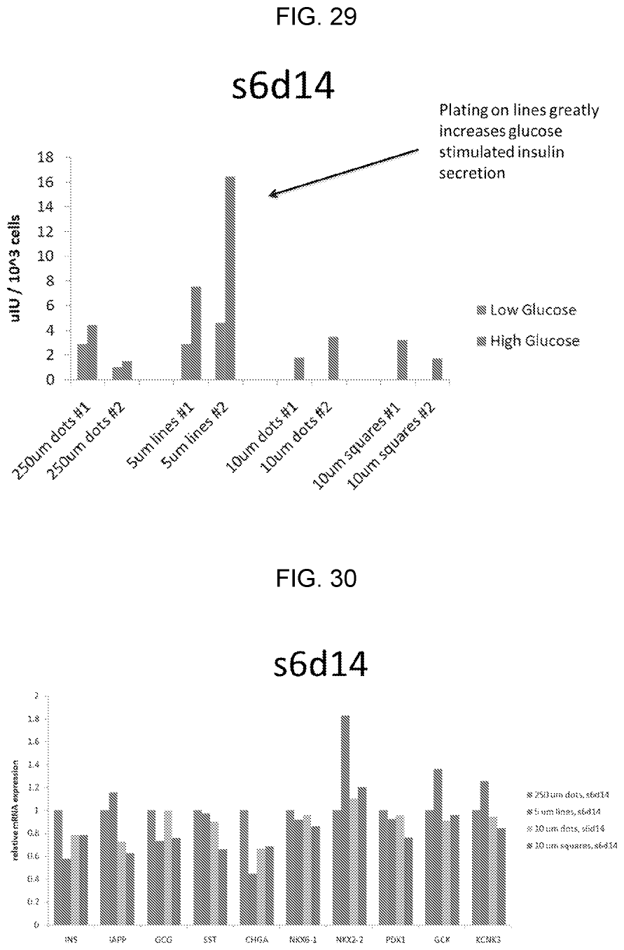

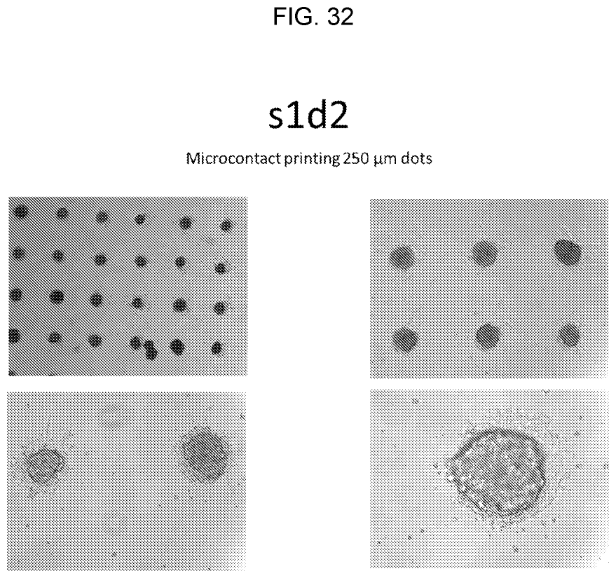

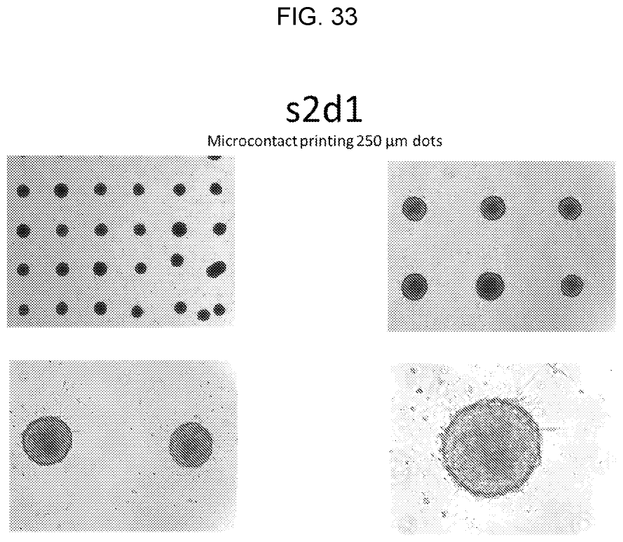

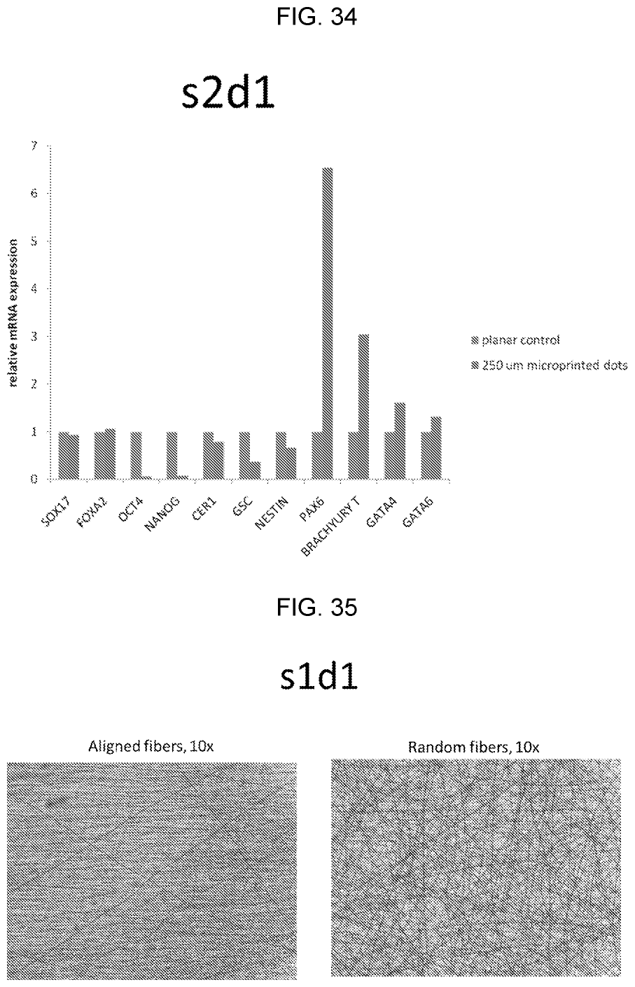

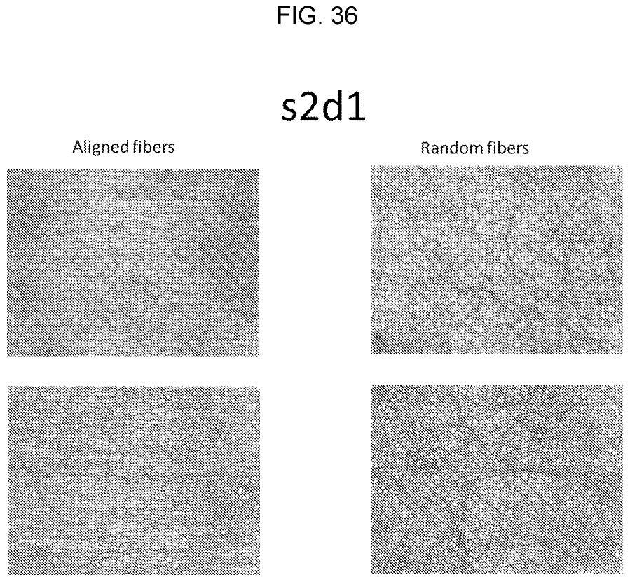

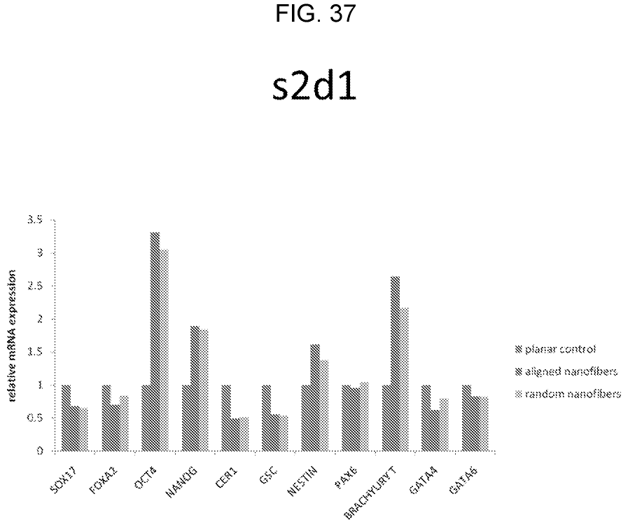

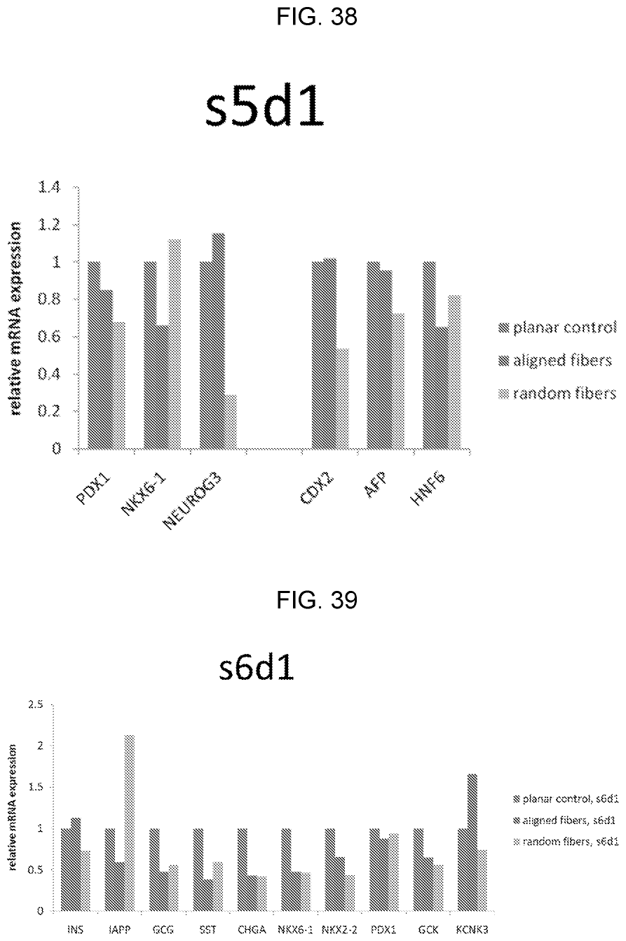

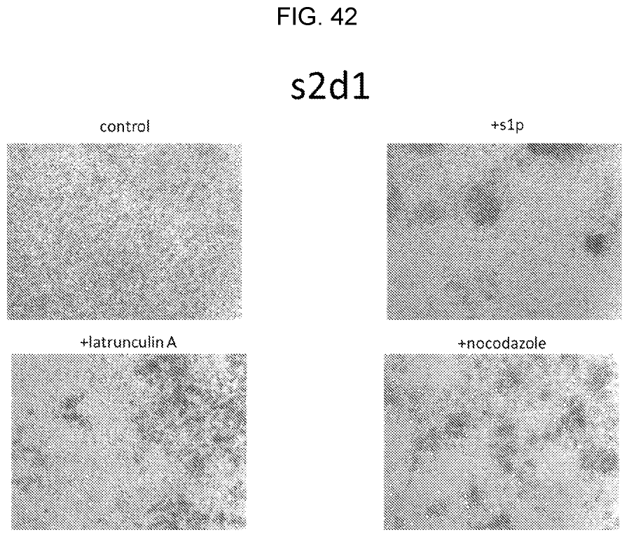

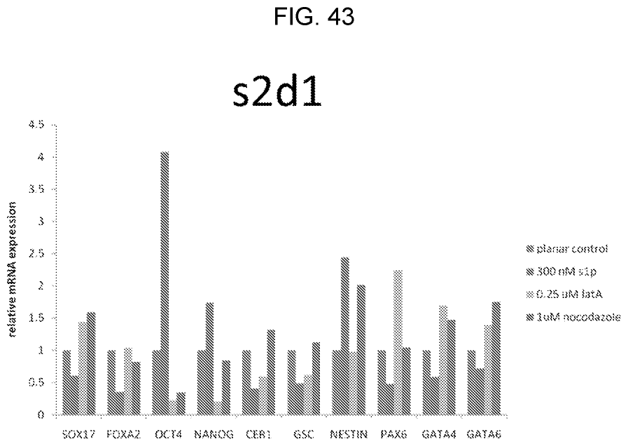

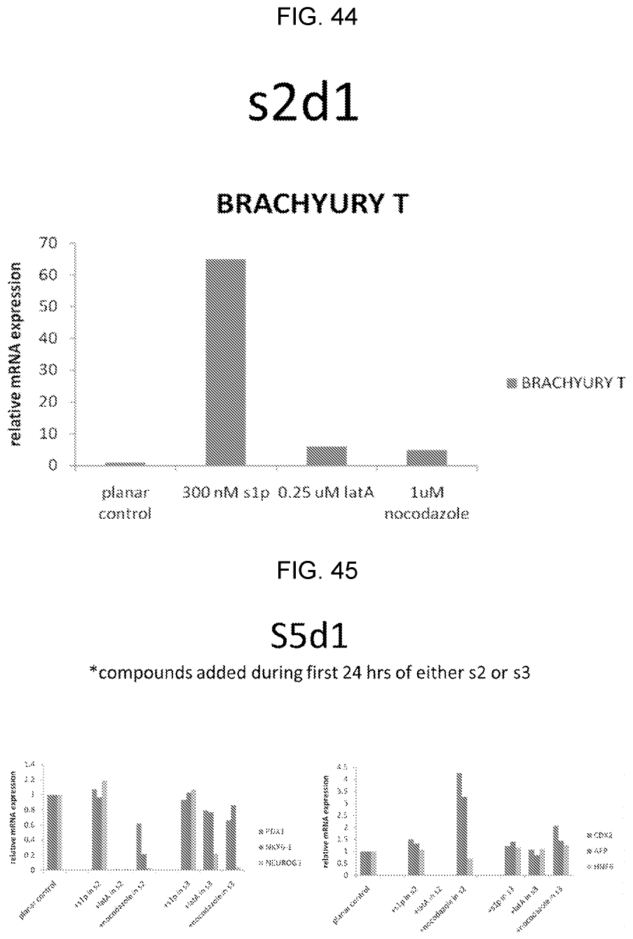

Controlling the physical microenvironment of cells through micropatterning, topography (electrospun fibers and suspension microcarriers), and substrate stiffness can strongly influence cell fate at various stages of the SC-β cell protocol. See e.g., FIG. 26-FIG. 49.

Modifying the cytoskeleton with soluble small molecules rather than microenvironmental cues at various stages of the protocol can also greatly influence cell fate. Controlling the physical microenvironment to improve differentiation and maturation of SC-β cells can lead to practical protocol improvements to increase function both in vitro and in vivo. Substrate parameters that improve SC-β cell function can be incorporated into device designs for transplantation to improve graft efficacy.

(a) Microcontact Printing (See e.g., FIG. 26-FIG. 30)

Planar Stage 6 Cells Dispersed on s6d7 and Replated on Microcontact Printed Patterns (Collagen I).

Patterning stem cells can strongly influence expression of genes associated with various germ layers. Plating on lines greatly increases glucose stimulated insulin secretion. In stage 6, cell shape and cytoskeletal arrangement don't necessarily increase traditional maturation genes but instead are important for the proper insulin secretion machinery (see e.g., FIG. 30).

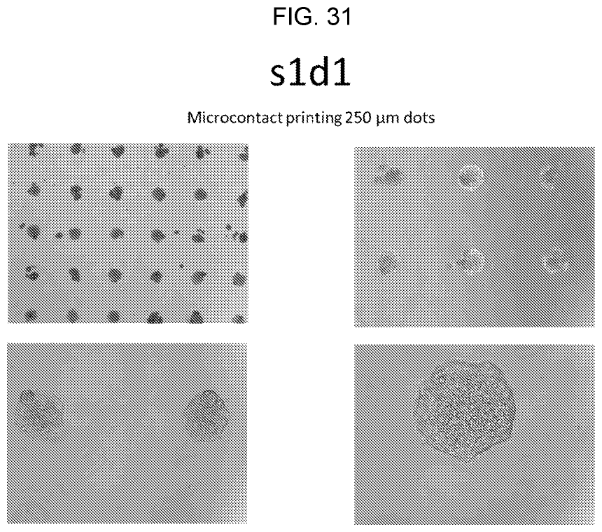

Stem Cells were Plated onto 250 μm Dots and Differentiated Through Stage 1 (See e.g., FIG. 31-FIG. 34).

Patterning stem cells can strongly influence expression of genes associated with various germ layers (see e.g., FIG. 31-FIG. 34).

(b) Electrospun Nanofibers (See e.g., FIG. 35-FIG. 39)

Stem cells were plated onto electrospun nanofibers and differentiated with the planar SC-β cell protocol. Changing substrate topography experienced by stem cells with electrospun fibers can strongly influence expression of genes associated with various germ layers. Later in the protocol, the fibers also influence genes associated with beta cells as well as other endodermal lineages.

Later in the protocol, the fibers also influence genes associated with beta cells as well as other endocrine cell types.

(c) Substrate Stiffness (See e.g., FIG. 40)

Stem cells were plated onto soft PDMS plates (0.2 kPa and 2 kPa) and differentiated through stage 1. Changing substrate stiffness experienced by stem cells can strongly influence expression of genes associated with various germ layers.

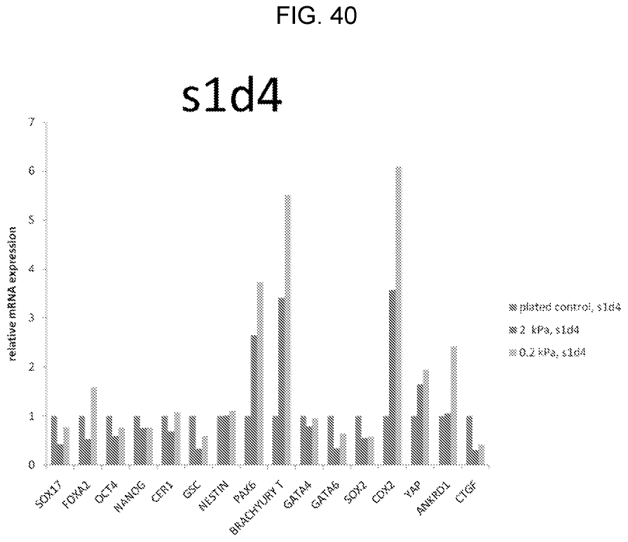

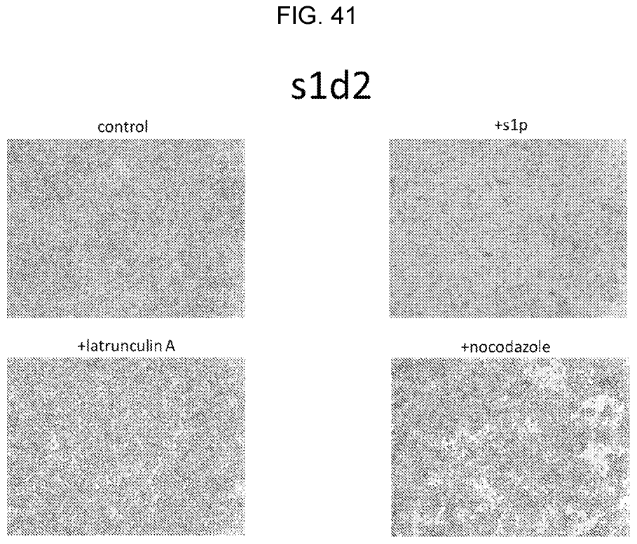

(d) Cytoskeletal Modulating Compounds (See e.g., FIG. 41-FIG. 45)

Small molecules that influence the state of the cytoskeleton were added for first 24 hours of various stages of the SC-β cell protocol.

Adding these cytoskeletal modulating compounds during stage 1 can strongly influence expression of genes associated with various germ layers.

Adding these cytoskeletal modulating compounds during stage 2 or 3 can strongly influence expression of genes associated with pancreatic progenitors as well as other endodermal lineages.

In particular, s1p (which induces actin polymerization) greatly increases the expression of the mesoderm marker Brachyury T.

Adding these cytoskeletal modulating compounds during stage 2 or 3 can strongly influence expression of genes associated with pancreatic progenitors as well as other endodermal lineages.

(e) Suspension Bead Microcarriers (See e.g., FIG. 46-FIG. 49)

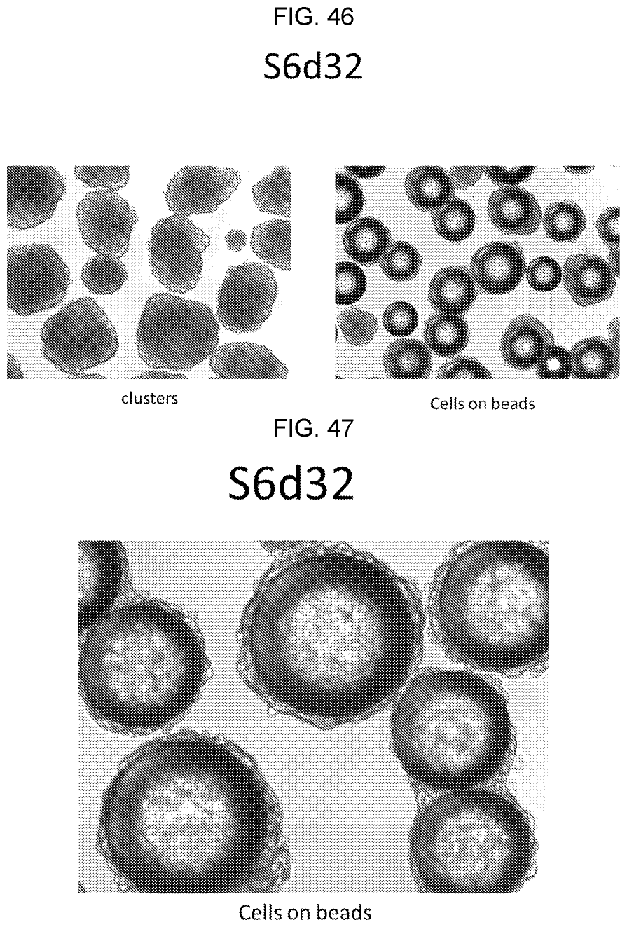

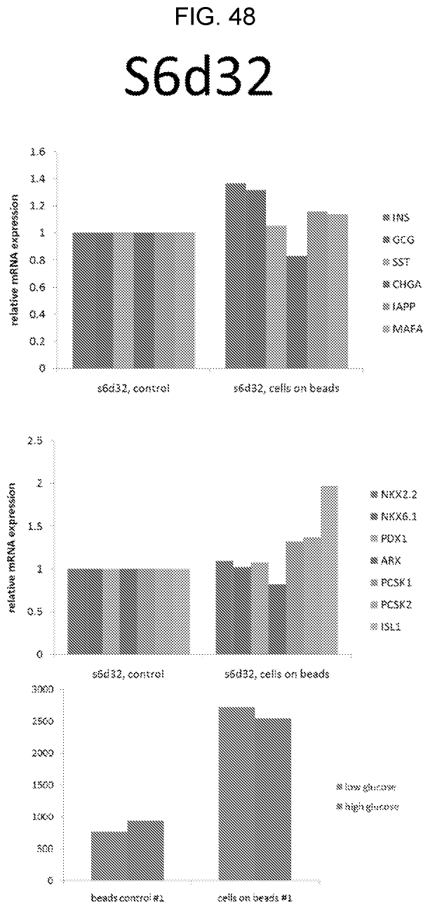

Stage 6 cells were single cell dispersed clusters at s6d20 and seeded onto matrigel-coated suspension beads. Stage 6 cells are able to attach to the surface of microcarriers (FIG. 47). Attaching stage 6 cells to microcarriers may influence SC-β cell gene expression and GSIS (FIG. 48). Undifferentiated stem cells can also be successfully attached and cultured on bead microcarriers in a bioreactor (FIG. 49).

(IV) How SC-β Cells Respond to Chemical and Genetic Stress (See e.g., FIG. 50-FIG. 55)

SC-β cells respond to chemical stress by:
Increasing ER stress marker protein and gene expression
Reduced function (GSIS)
Reduced cellular respiration

SC-β cells respond to genetic stress induced by diabetes-causing gene mutations through:
Reduced function
Increased ER stress marker protein and gene expression
Reduced function (sGSIS and dGSIS), insulin content
Reduced cellular respiration
Increased proinsulin/insulin ratio
Inability to control blood glucose in diabetic mice
Differentiating into multiple non-pancreatic cell types

This methodology and the characterization assays describe a roadmap to evaluate the effect of chemical and/or genetic stress on SC-islets, specifically SC-β cells.

SC-β cells respond to chemical stress: increased ER stress gene expression, increased ER stress proteins, and reduced glucose stimulated insulin secretion (see e.g., FIG. 50).

SC-β cells with diabetes-causing mutations respond to genetic stress in vitro and in vivo: reduced glucose stimulated insulin secretion, reduced insulin content, and increased proinsulin/insulin ratio (see e.g., FIG. 51).

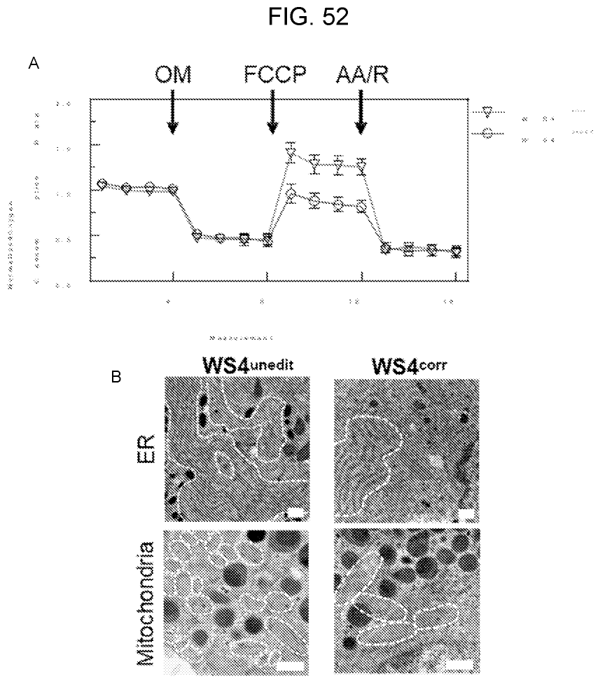

SC-β cells with diabetes-causing mutations respond to genetic stress in vitro: reduced maximal respiratory capacity and swollen ER and fragmented mitochondria (see e.g., FIG. 52).

SC-β cells with diabetes-causing mutations respond to genetic stress in vivo: unable to regulate glucose and reduced glucose stimulated insulin secretion (see e.g., FIG. 53).

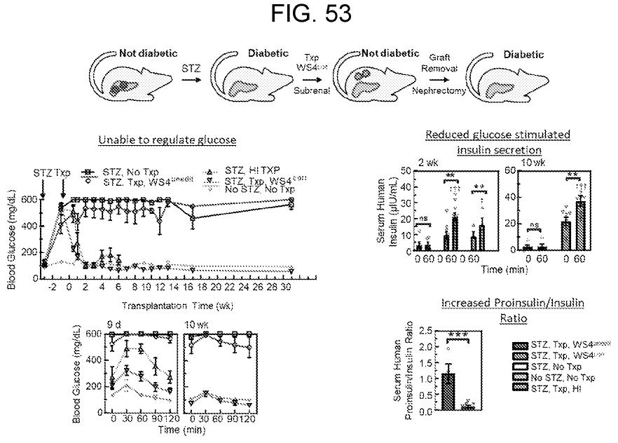

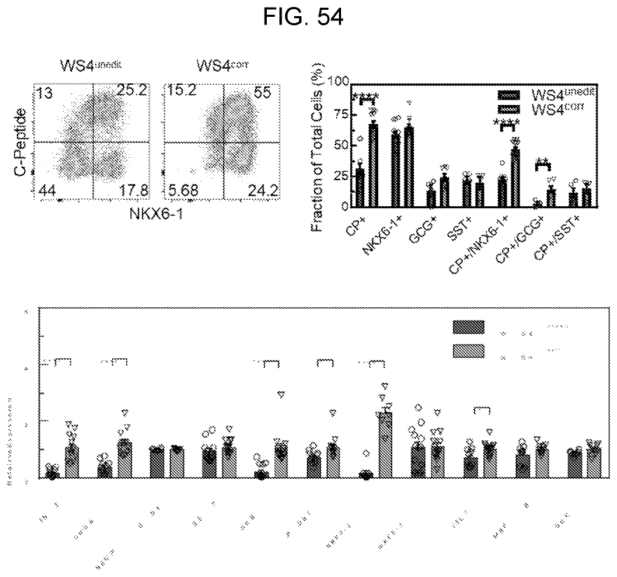

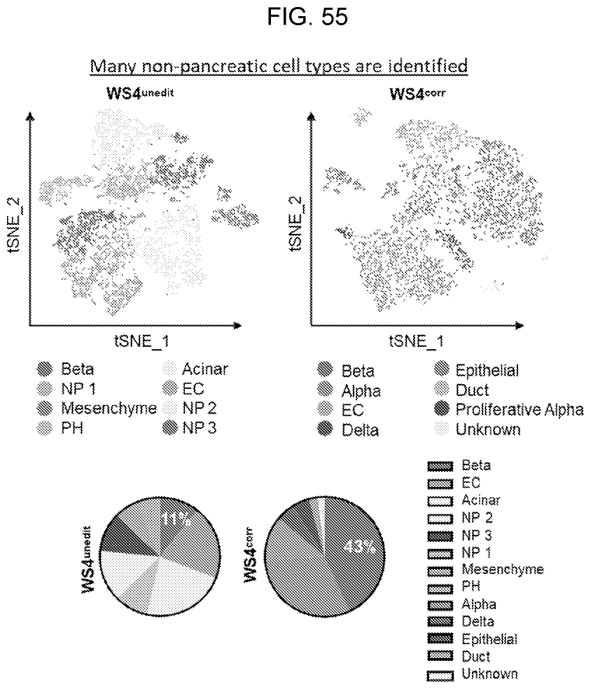

Genetically stressed SC-islets produce non-pancreatic cell types from 6 stage differentiation protocol. Reduction in SC-β yields was observed from SC-islet differentiation (see e.g., FIG. 54) and many non-pancreatic cell types are identified (see e.g., FIG. 55).

(V) Cell Hashing Stressed β Cells

Islets respond to multiple forms of chemical stress (cytokines, calcium modulators, protein folding inhibitors) by increasing ER stress gene expression.

Islets can be exposed to chemical stress, single cell dispersed, and tagged with hashing antibodies to enable single cell RNA sequencing of multiple conditions simultaneously on a single sequencing lane.

ER stress occurs in all forms of diabetes, therefore applying chemicals to induce ER stress in cadaveric human islets will elucidate key pathways that are upregulated when different cell modulators effect protein folding and apoptosis is induced. Cell hashing allows for simultaneously evaluating multiple chemical stressors on primary islet cell types at a lower cost by tagging each sample with antibodies that can be divided after sequencing.

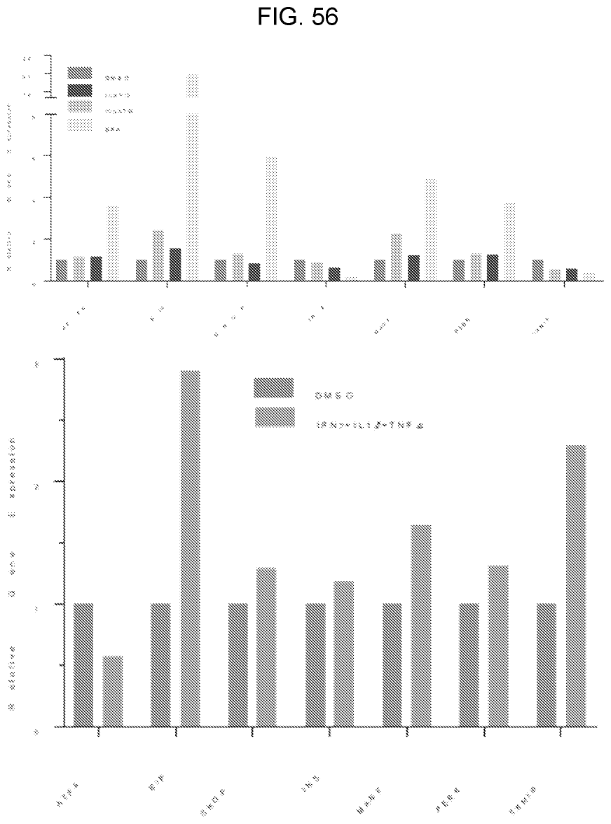

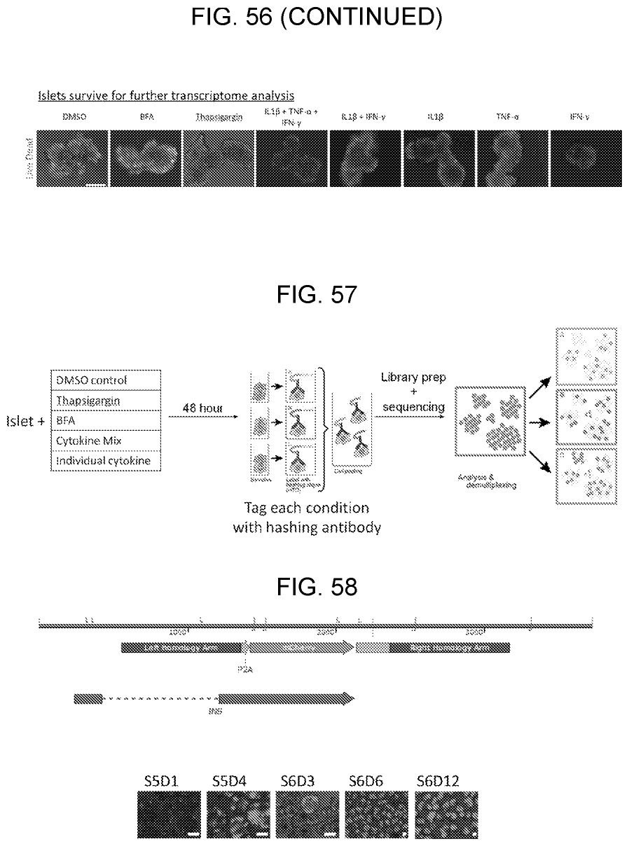

Cadaveric Human Islets with Stress show increased ER stress gene expression (see e.g., FIG. 56). FIG. 56 also shows islets survive for further transcriptome analysis. Hashing of stressed cadaveric human islets is shown in FIG. 57. Cell Hashing is a method that enables sample multiplexing and super-loading on single cell RNA-sequencing platforms. Cell Hashing uses a series of oligo-tagged antibodies against ubiquitously expressed surface proteins with different barcodes to uniquely label cells from distinct samples, which can be subsequently pooled in one scRNA-seq run. By sequencing these tags alongside the cellular transcriptome, we can assign each cell to its sample of origin, and robustly identify doublets originating from multiple samples.

(VI) Using mCherry/INS Reporter Cell Line to Study β Cell Health (See e.g., FIG. 58-FIG. 61)

Stage 6 INS+/−mcherry SC-islets can be single cell dispersed, sorted for INS+ SC-β cells, and treated with a SERCA pump inhibitor (thapsigargin) to reduce insulin secretion for high throughput drug screening.

The reduction of mCherry/INS expression can be quantified and used to understand SC-β cell health. Prolonged thapsigargin exposure can reduce mCherry fluorescence (insulin expression).

This method allows for high throughput drug screening.

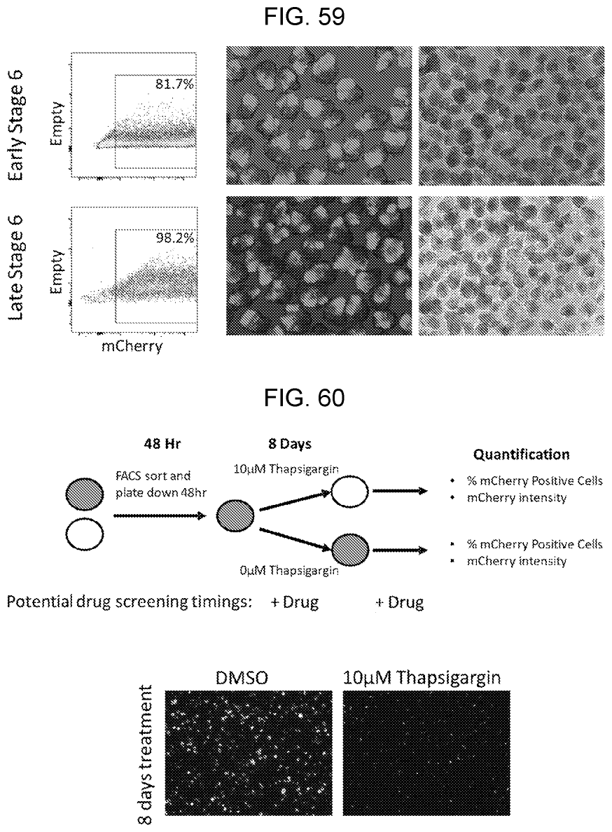

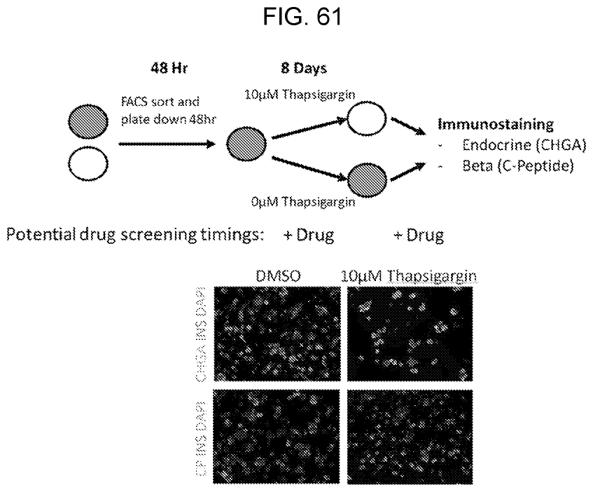

mCherry fluorescence increases at late stages of SC-islet differentiation (FIG. 58-59). INS-mCherry+ cells with thapsigargin treatment reduces mCherry fluorescence (FIG. 60). De-differentiation signatures occur in INS-mCherry+ cells with thapsigargin treatment (FIG. 61).

(VII) Refining Stage 6 Enriched Serum-Free Media (ESFM) for SC-β Cells

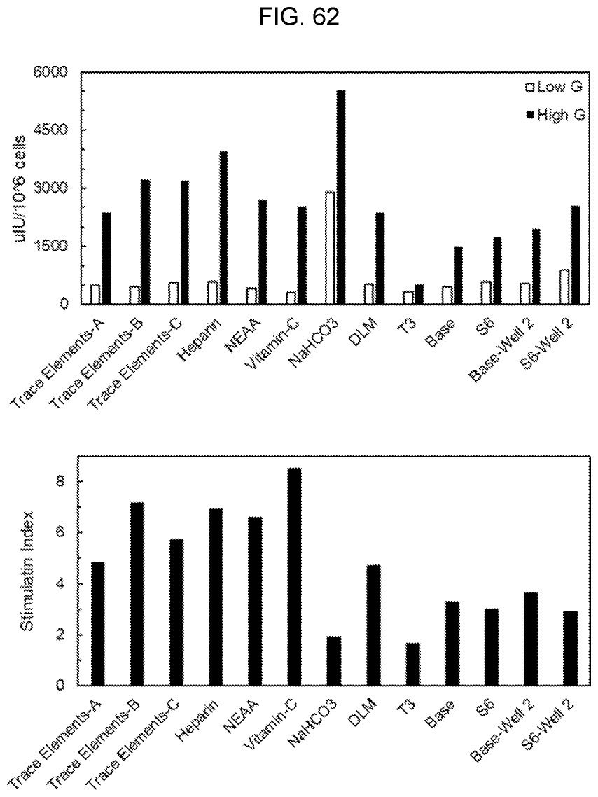

NAHCO₃ in ESFM reduces stimulation index by increasing insulin secretion at low glucose. Defined Lipid Mixture at 1:100 reduced stimulation index by increasing insulin secretion at low glucose. Data could indicate that s6 factors may be more beneficially when added individually than combined together. Refining stage 6 ESFM could help increase maturation and function of SC-β cells. NAHCO₃ in ESFM reduces stimulation index by increasing insulin secretion at low glucose (FIG. 62). Adding individual components on top of base media increases stimulation GSIS.

Base Media
MCDB131
10% BSA
Glutamax
P/S
Glucose
Zn 10 μM

Treatment on Top of Base Media
1-Trace A 1:1000
2-Trace B 1:1000
3-Trace C 1:1000
4-Heparin 10 μg/mL
5-NEAA 1:100
6-Vitamin C 250 μM
7-NaHCO₃20 mM
8-Defined Lipid Mixture 1:1000
9-T3 1 μM

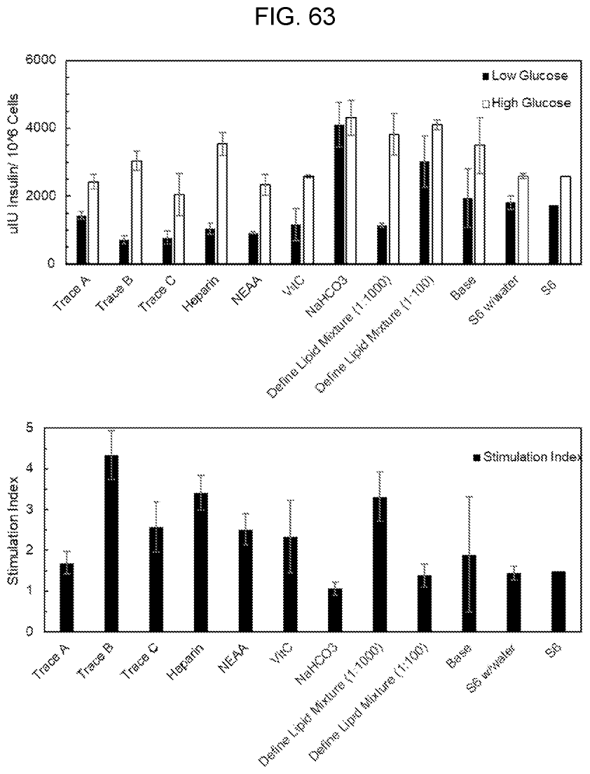

Defined Lipid Mixture at 1:100 reduced stimulation index by increasing insulin secretion at low glucose (FIG. 63).

Base Media
MCDB131
10% BSA
Glutamax
P/S
Glucose
Zn 10 μM

Treatment on Top of Base Media
1-Trace A 1:1000
2-Trace B 1:1000
3-Trace C 1:1000
4-Heparin 10 μg/mL
5-NEAA 1:100
6-Vitamin C 250 μM
7-NaHCO₃20 mM
8-Defined Lipid Mixture 1:1000
9-Defined Lipid Mixture 1:1000
10-Base
11-S6 w/water dilution 1:100
12-S6 no water dilution

All samples had same total dilution of 1:100 with water to maintain same osmolarity amongst samples. Sample 12 did not have this dilution.

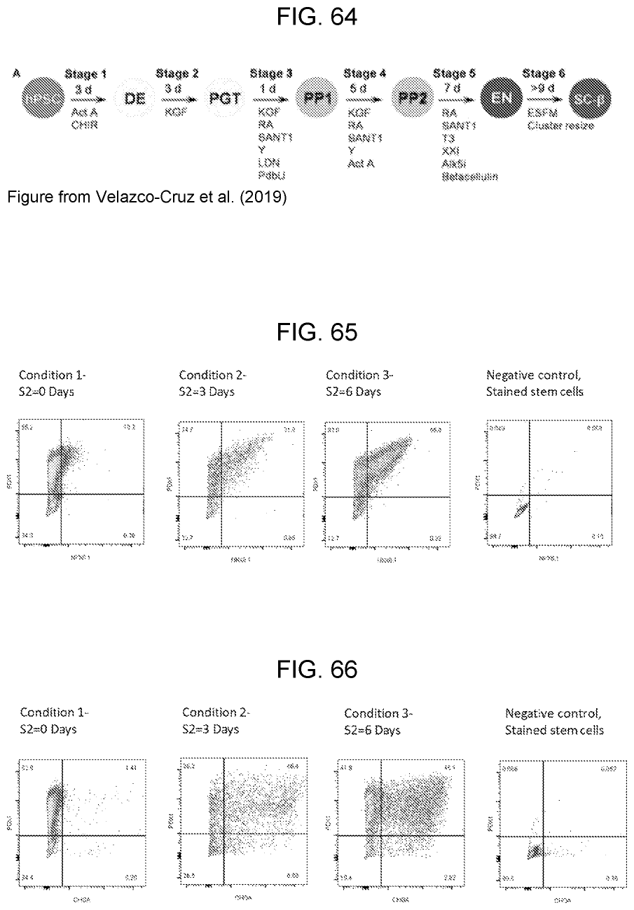

(VIII) Stage 2 Duration Affects Pancreatic Differentiation (FIG. 64-FIG. 70)

Changing the duration of stage 2 in the SC-beta cell differentiation protocol, which uses KGF to generate primitive gut tube, changes differentiation efficacy to pancreatic cells. Here is shown the refining of stage 2 differentiation to SC-β cells, other SC-islet cells, or other endodermal lineages.

Does the Duration of Stage 2 have any Effect on In-Suspension Differentiation of HUES8 hESC into Pancreatic Progenitors?

This experiment was done in 5 ml biotts using Velazco-cruz et al. 2019's in-suspension differentiation protocol (Velazco-cruz 2019 incorporated by reference). Cells were collected after Pancreatic Progenitors (PP2) were generated.

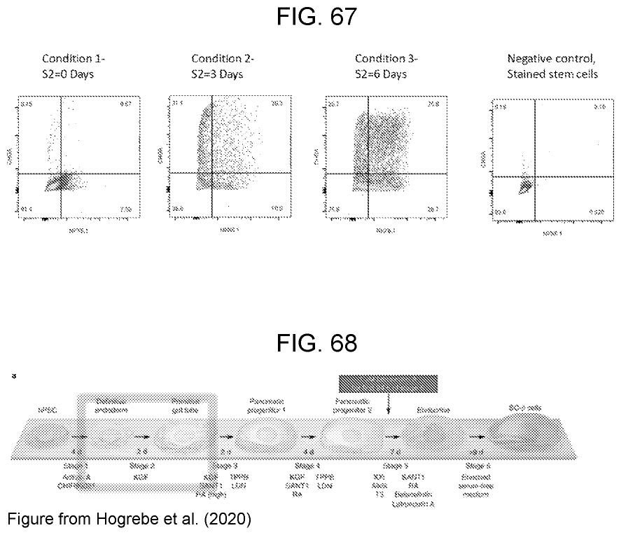

Scheme was modified from Velazco-cruz 2019 (see FIG. 64). 6 days of Stage 2 increases the number of PDX1+, NKX6.1+, and PDX1+/NKX6.1+PP2's (FIG. 65). 6 days of Stage 2 slightly decreases the number of CHGA+ and PDX1+/CHGA+PP2's (FIG. 66). 6 days of Stage 2 slightly increases the number of CHGA⁺/NKX6.1⁺ PP2's (FIG. 67).

Extending stage 2 duration positively impacts the expression of some pancreatic progenitor markers. The low number of cells generated using the 5 mL biotts, could explain some of the slight differences observed between 3 and 6 days of stage 2.

Pilot Experiment 2: Does the Duration of Stage 2 have any Effect on Planar Differentiation of HUES8 hESC into Pancreatic Progenitors?

The purpose of testing the effects of Stage 2's duration with Hogrebe et al. (2020)'s planar differentiation protocol was to avoid the limitation of small cell number faced with in-suspension differentiation with 5 mL biotts (see e.g., FIG. 68).
Condition 1→0 days of Stage 2 (Negative Control)
Condition 2→2 days of Stage 2 (Positive Control)
Condition 3→4 days of Stage 2
Condition 4→6 days of Stage 2
Condition 5→8 days of Stage 2
Condition 6→10 days of Stage 2
Collected PP2 and EN (pancreatic endocrine) cells.

We opted for more time points to ensure that we reached a time where cells would not differentiate properly.

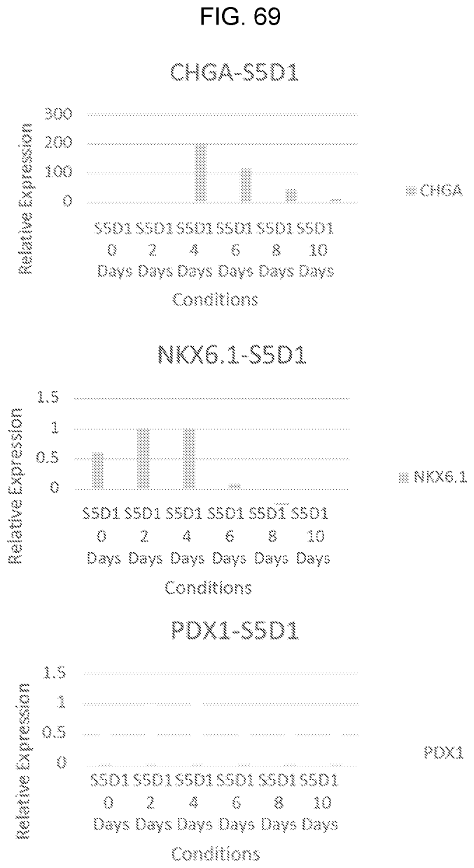

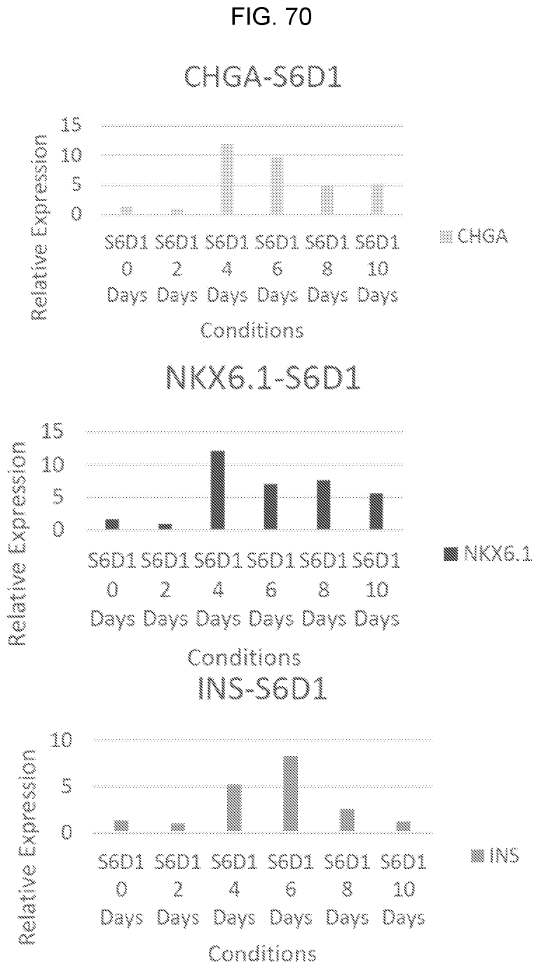

4 days of Stage 2 is optimal for CHGA but not NKX6.1 and PDX1 gene expression in PP2 cells (see e.g., FIG. 69). 4 days of Stage 2 increases CHGA, NKX6.1, and INS gene expression in EN cells (see e.g., FIG. 70).

Here it is shown that extending stage 2 to four days positively impacts the differentiation of HESC into pancreatic progenitors and endocrine progenitors.

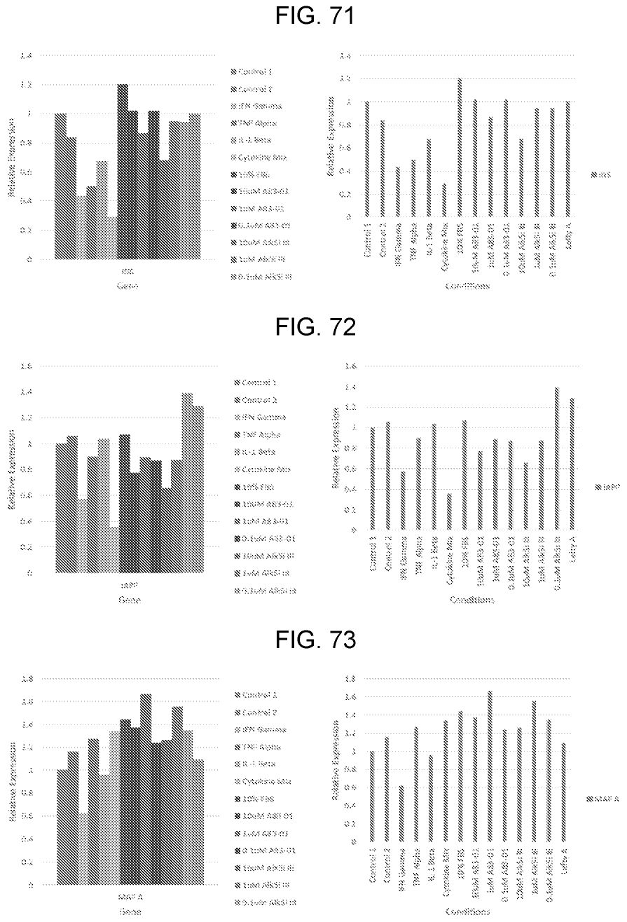

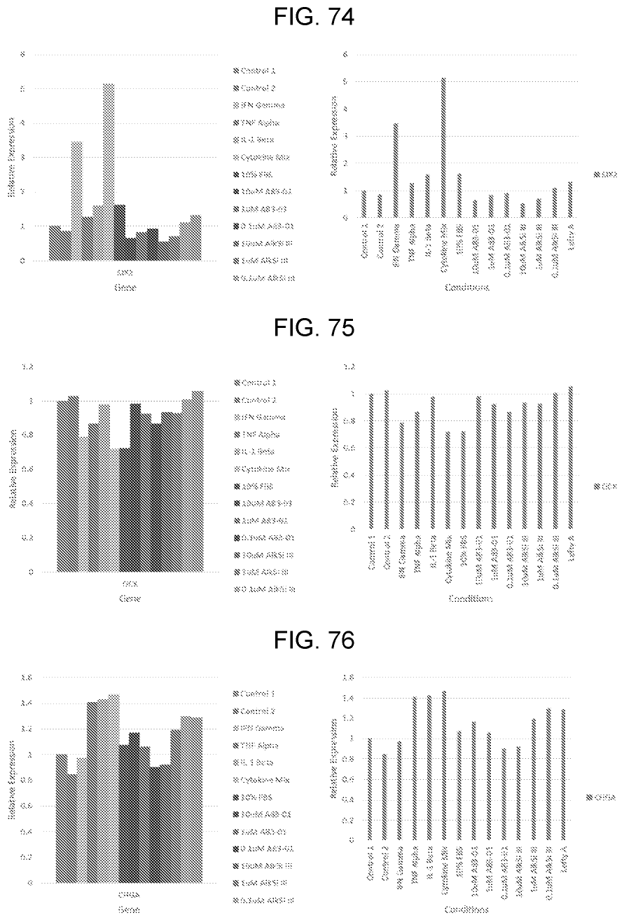

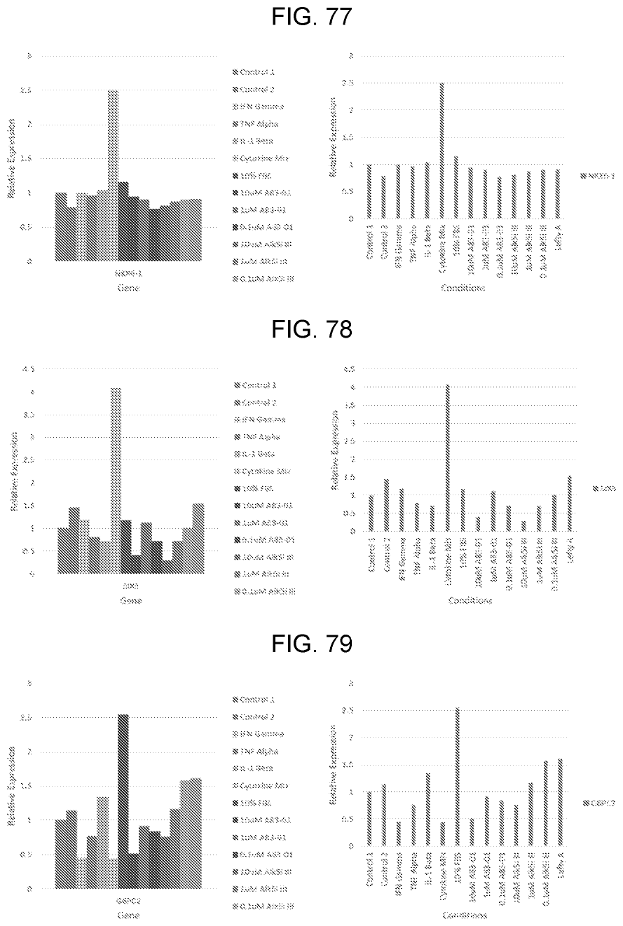

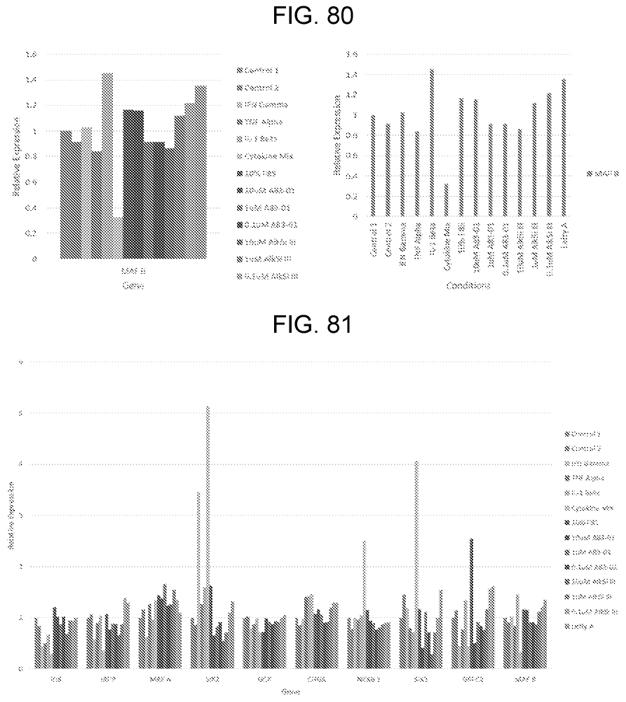

(IX) Compounds to Improve SC-Beta Cells (See e.g., FIG. 71-FIG. 82)

10% FBS increased expression of 7/10 genes—INS, MAF A, SIX2, NKX6-1, SIX3, G6PC2, and MAF B. It slightly decreased GCK expression.

1 μg/mL Lefty A increased expression of 6/10 genes-IAPP, SIX2, CHGA, SIX3, G6PC2, and MAF B. It slightly decreased NKX6-1 expression.

0.1 μM Alk5i III increased expression of 5/10 genes-IAPP, MAF A, CHGA, G6PC2, and MAF B. Slightly decreased INS and NKX6-1 expression.

Further compounds in stage 6 could improve differentiation, function, and maturation of SC-beta cells.

Background

Cultured 14 wells of 6-well plates containing Stage 6 SC-Beta cells differentiated in suspension by Punn. Two wells served as controls.

Each well was cultured in one of the following for 7 days*:
360 ng/μL IFN Gamma
50 ng/μL TNF Alpha
10 ng/μL IL-1 Beta
Mixture of the above cytokines
10% FBS
10, 1, and 0.1 μM A83-01
10, 1, and 0.1 μM Alk5i Ill
1 μg/mL Lefty A (*cultured for 6 days)

Ran qPCR and compared everything to Control 1 (had smallest Ct SD).

(X) ECM Proteins and Stiffness Influence SC-Beta Cell Function and Maturation

Plating SC-beta cells improves them. SC-beta cell improvement is controlled by stiffness. ECM protein concentration and stiffness work synergistically to improve SC-beta cells. These modulations to stage 6 could improve differentiation, function, and maturation of SC-beta cells.

Modifications to Protocol

ECM related modifications:
Plating down SC beta cells
Varying Matrigel concentration on plate down SC beta cells
Changing ECM molecules for SC beta cell plate down
Varying stiffnesses
Increasing ECM molecules on softer substrate for SC beta cell plate down
Different ECM for planar differentiation

Improvement in Gsis (Glucose Stimulated Insulin Secretion) and Maturation Genes in Plated SC Beta Cells on Matrigel

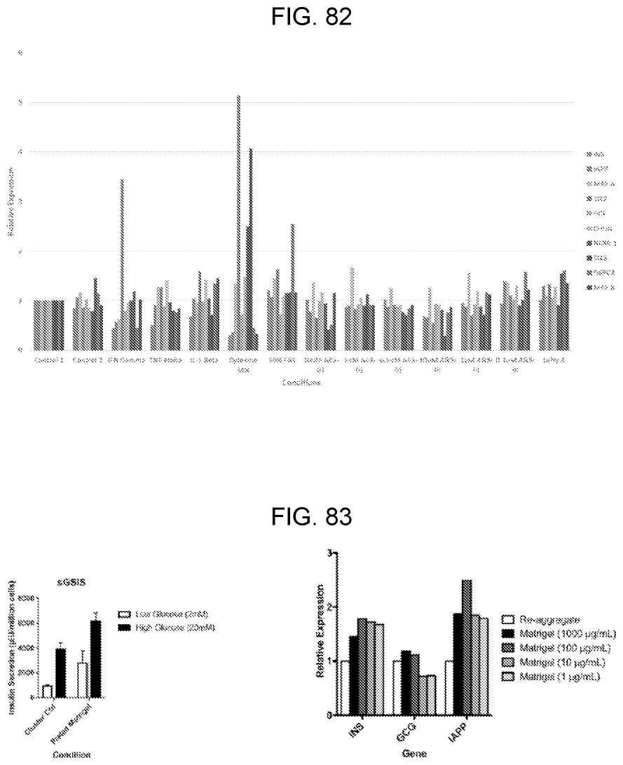

FIG. 83 shows improvement in GSIS by plating down SC beta cells. sGSIS, insulin content, and immunohistochemistry show maturation proteins. Here we show that SC beta cells interacting with Matrigel (which is a mixture of multiple ECM components) can have improved effects on insulin release and genes.

Plating Down with Different Matrigel Coating Condition (See e.g., FIG. 84)

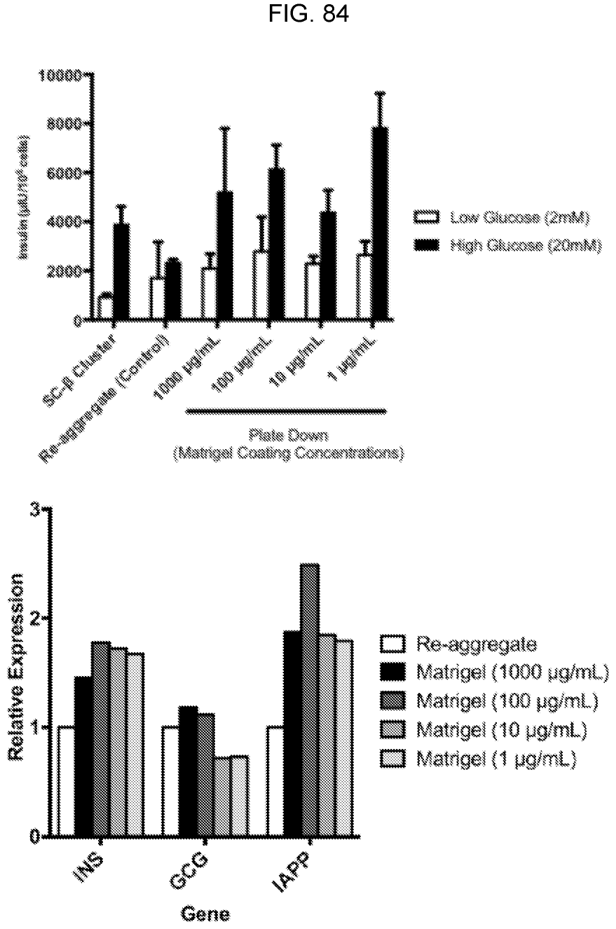

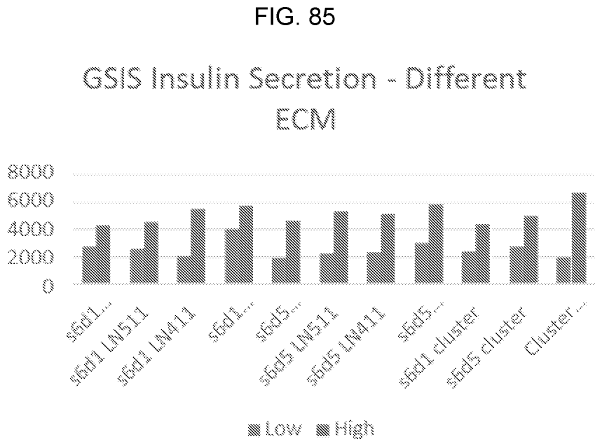

Improvements in gsis and maturation genes do not depend on specific ECM or adhesion molecules (see e.g., FIG. 85). Mechanical properties (stiffness) influence SC beta cell gsis performance and maturation (increased stiffness results in increased gsis) (see e.g., FIG. 86).

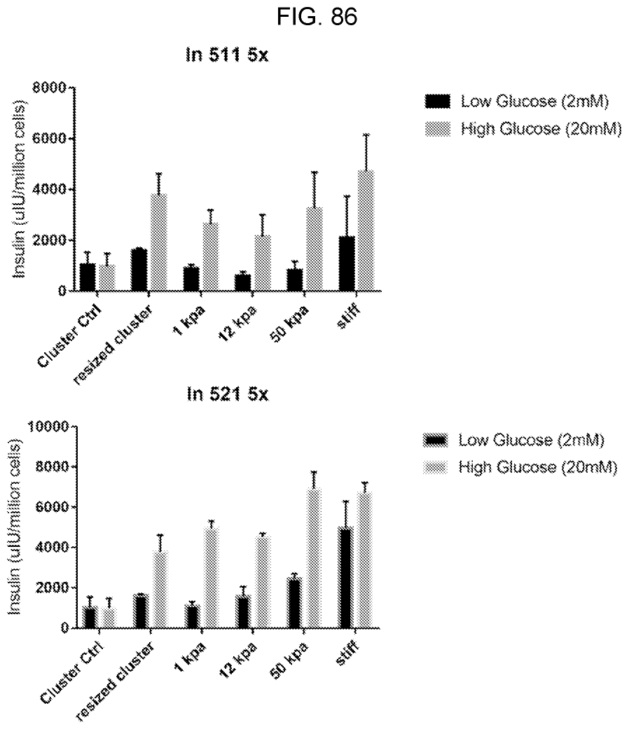

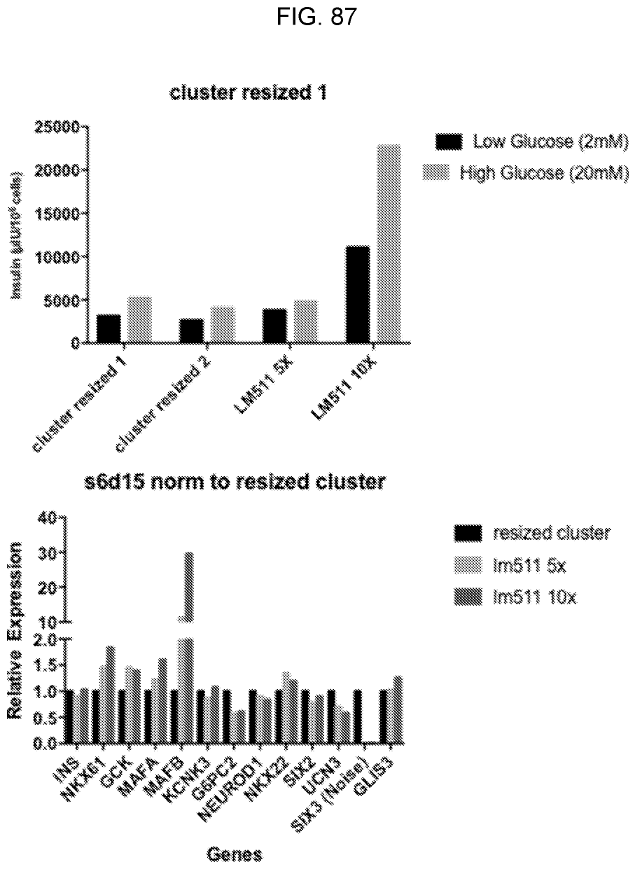

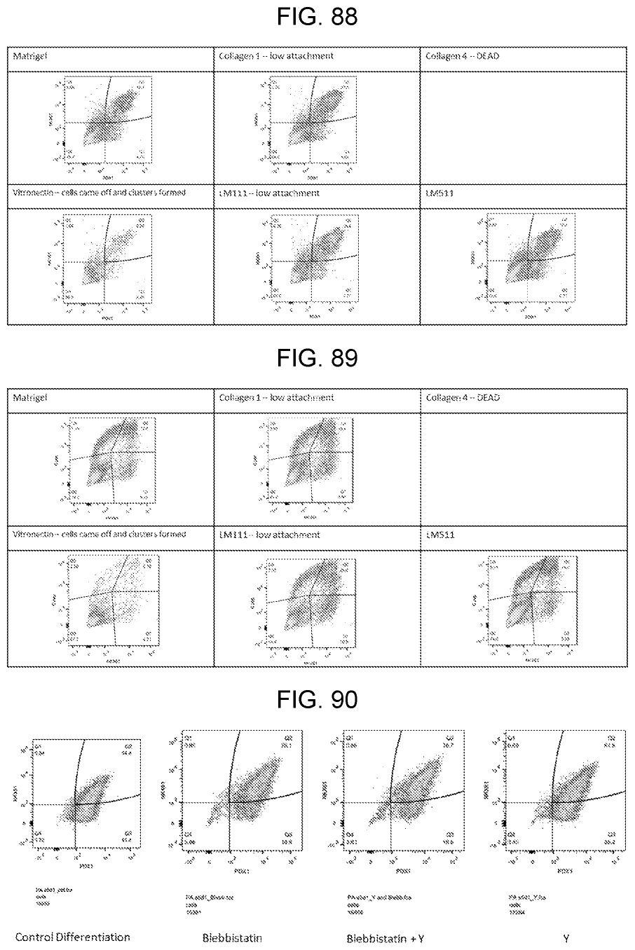

Increasing extracellular matrix (ECM) protein concentration matures SC beta cells on softer substrate (25 kPa) (see e.g., FIG. 87). Different ECMs used for planar differentiation (e.g., matrigel, collagen I, Vitronectin, LM111, LM511) (see e.g., FIG. 88-FIG. 89).

(XI) Other Modifications to the Protocol

Other Modifications with Benefits (See e.g., FIG. 90-FIG. 95).
Y and Blebbistatin treatment during stage 4 of differentiation
Reducing volume of media
Wnt modification—IWP2 treatment during s1d2-d4
bFGF treatment during stage 1
Betacellulin removal during S5
Other compound treatments during Stage 6

These modulations could improve differentiation, function, and maturation of SC-beta cells.

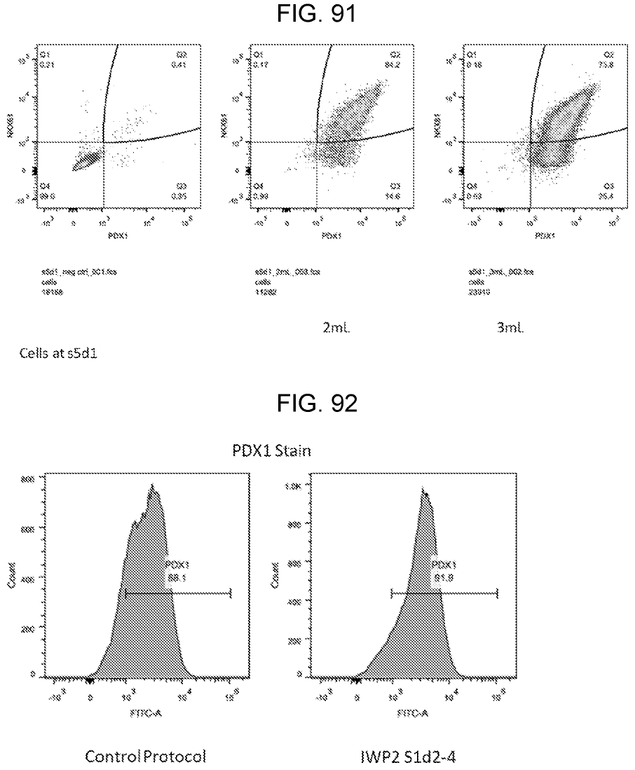

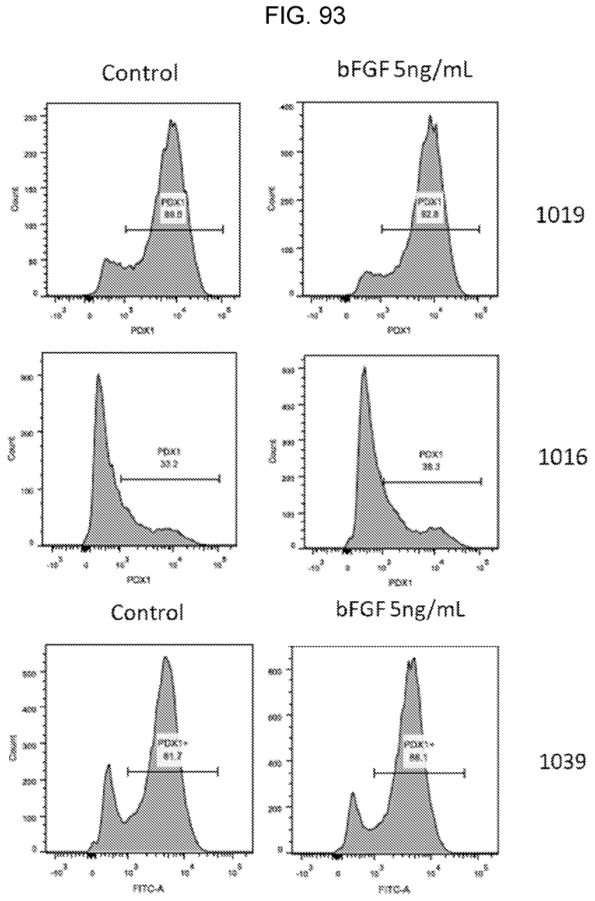

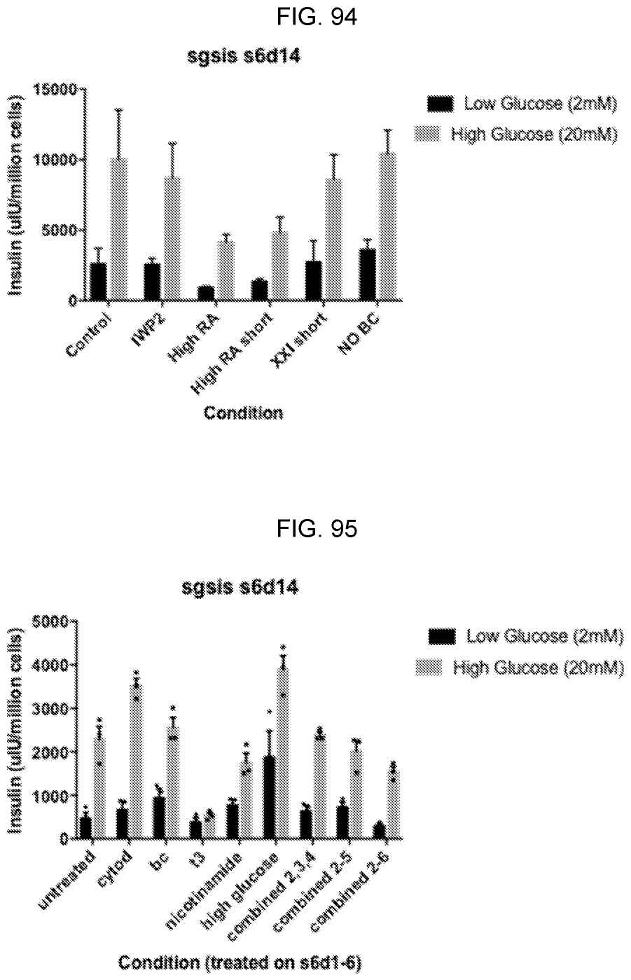

Y and Blebbistatin treatment during S4 Improves PDX1/NKX61 co expression (see e.g., FIG. 90). Reduced volume (e.g., 2 ml, 3 ml) maintains good SC beta cell differentiation (see e.g., FIG. 91). IWP2 during Sd2-d4 promotes PDX1 yield at S3 (see e.g., FIG. 92). bFGF treatment during Stage 1 improves differentiation (see e.g., FIG. 93). BC not required for Beta cell induction (see e.g., FIG. 94). CytoD or High Glucoses treatment during s6d1-7 increases insulin secretion (see e.g., FIG. 95).

1. A method of generating insulin-producing beta cells in a suspension comprising:
(Stage 1) providing a stem cell;
providing a serum-free media; and
contacting the stem cell with a TGFβ/Activin agonist or a glycogen synthase kinase 3 (GSK) inhibitor or WNT agonist for an amount of time sufficient to form a definitive endoderm cell;
(Stage 2) contacting the definitive endoderm cell with a FGFR2b agonist for an amount of time sufficient to form a primitive gut tube cell;
(Stage 3) contacting the primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell;
(Stage 4) incubating the early pancreas progenitor cell for at least about 3 days and optionally contacting the early pancreas progenitor cell with a rho kinase inhibitor, a TGF-β/Activin agonist, a smoothened antagonist, an FGFR2b agonist, or a RAR agonist for an amount of time sufficient to form a pancreatic progenitor cell; or
(Stage 5) contacting the pancreatic progenitor cell with an Alk5 inhibitor, a gamma secretase inhibitor, SANT1, Erbb1 (EGFR) or Erbb4 agonist, or a RAR agonist for an amount of time sufficient to form an endoderm cell; and
(Stage 6) allowing the endoderm cell to mature in a second serum-free media for an amount of time sufficient to form a beta cell.

2. The method of claim 1, wherein the beta cell is a plurality of beta cells and are re-aggregated into clusters after single cell dispersing and seeding into spinner flasks.

3. The method of claim 1, wherein beta cells are single cell dispersed, cryopreserved, and thawed, and retain function and marker expression.

4. The method of claim 1, wherein the environments of stage 1-stage 6 cells are modulated via controlling the physical microenvironment of cells through micropatterning topography, substrate stiffness, modifying the cytoskeleton with soluble small molecules.

5. The method of claim 1, wherein the beta cells are planar dispersed on about day 7 and replated on microcontact printed patterns.

6. The method of claim 1, wherein the stem cells were plated onto micron-sized dots and differentiated through stage 1.

7. The method of claim 1, wherein the stem cell is a plurality of stem cells and are plated onto electrospun nanofibers and planar differentiated.

8. The method of claim 1, wherein the stem cell is a plurality of stem cells and are plated onto soft PDMS plates (about or between about 0.2 kPa or about 2 kPa) and differentiated through stage 1.

9. The method of claim 1, comprising adding cytoskeletal modulating compounds during stage 1, 2, or 3.

10. The method of claim 1, wherein hPSCs or SC-beta cells are attached and cultured on bead microcarriers in a bioreactor.

11. The method of claim 1, comprising adding an auxiliary component to ESFM base media (in stage 6), wherein the auxiliary component is capable of modulating GSIS stimulation selected from one or more of:
Trace A;
Trace B;
Trace C;
Heparin;
NEAA;
Vitamin C;
NaHCO₃;
Defined Lipid Mixture;
Defined Lipid Mixture;
Base; or
T3.

12. The method of claim 1, wherein an amount of time sufficient to form a primitive gut tube cell (in stage 2) is about 6 days and results in an increased number of PDX1⁺, NKX6.1⁺, and PDX1⁺/NKX6.1⁺ PP2 cells; a decreased number of CHGA⁺ and PDX1⁺/CHGA⁺ PP2 cells; or an increased number of CHGA⁺/NKX6.1⁺ PP2 cells.

13. The method of claim 1, wherein an amount of time sufficient to form a primitive gut tube cell (in stage 2) is about 4 days and results in increased CHGA, NKX6.1, and INS gene expression in EN cells.

14. The method of claim 1, wherein the second serum-free media comprises 10% FBS, which results in increased expression of INS, MAF A, SIX2, NKX6-1, SIX3, G6PC2, and MAF B, and decreased GCK expression.

15. The method of claim 1, wherein the second serum-free media comprises Lefty A, which results in increased expression of IAPP, SIX2, CHGA, SIX3, G6PC2, and MAF B and decreased NKX6-1 expression.

16. The method of claim 1, wherein the second serum-free media comprises 0.1 μM Alk5i III increased expression of IAPP, MAF A, CHGA, G6PC2, and MAF B and decreased INS and NKX6-1 expression.

17. The method of claim 1, comprising plating a plurality of SC-beta cells.

18. The method of claim 1, comprising plating a plurality of SC-beta cells on a stiff substrate or a soft substrate.

19. The method of claim 1, comprising modulating extracellular matrix (ECM) protein concentration and stiffness to improve SC-beta cells.

20. The method of claim 1, comprising Y (Y27632) and Blebbistatin treatment during stage 4 of differentiation.

21. The method of claim 1, comprising reducing volume of media.

22. The method of claim 1, comprising Wnt treatment modification.

23. The method of claim 1, comprising bFGF treatment during stage 1, results in improved differentiation.

24. The method of claim 1, comprising Betacellulin removal during stage 5.

25. The method of claim 1, comprising IWP2 treatment during stage 2 day 4, resulting in an increase of PDX1 yield at S3.

26. The method of claim 1, wherein the second serum-free media in stage 6 does not comprise BC.

27. The method of claim 1, comprising CytoD treatment or high glucose treatment during stage 6, days 1-7 and results in an increase of insulin secretion.

28. A method of evaluating genetic stress of a cell comprising:
providing a cell from a subject, wherein if the cell forms a non-pancreatic cell type using the 6 stage differentiation protocol, or an optimization thereof, the cell is genetically or chemically stressed.

29. A method of evaluating chemical stress of a cell comprising:
providing an islet cell, exposed to chemical stress, single cell dispersed, and tagged with hashing antibodies to enable single cell RNA sequencing of multiple conditions simultaneously on a single sequencing lane.

30. A method of hashing stressed islet cells comprising: providing a human islet cell and incubated for a time sufficient to form cells sufficient for tagging, tagging each condition with a hashing antibody, and detecting the hashing antibodies.

31. A method of high-throughput drug screening or measurement of beta cell health comprising: providing a stage 6 INS+/−mcherry SC-islet, single cell dispersing the SC islet, sorting for INS+ SC-β cells, wherein if a reduction of mCherry/INS expression correlates with SC-β cell health.

32. A method of high-throughput drug screening comprising:
providing a stage 6 INS+/−mcherry SC-islet, single cell dispersing the SC islet, sorting for INS+ SC-β cells; and optionally
treating with a SERCA pump inhibitor, which results in a reduction in insulin secretion for high throughput drug screening.

33. A method of treating diabetes in a subject comprising transplanting stem cell-derived β cells CRISPR/Cas9-corrected for a diabetes-causing gene variant in WFS1 to restore glucose homeostasis.

34. The method of claim 19, further comprising plating down SC beta cells; varying matrigel concentration (improved effects on insulin release and genes) on plate down SC beta cells; changing ECM molecules for SC beta cell plate down; varying stiffnesses (increases in stiffness results in gsis performance and SC beta cell maturation); increasing ECM molecules on softer substrate (increasing ECM concentration matures SC beta cells on softer substrate) for SC beta cell plate-down; or different ECM for planar differentiation; or combinations thereof.


SEQUENCE LISTING (106 sequences)

SEQ ID NO: 1 - DNA, 23 bp, Artificial Sequence
  Synthetic CRISPR sequence
gaccatggagctccacgtcaggg

SEQ ID NO: 2 - DNA, 123 bp, Artificial Sequence
  Synthetic CRISPR sequence
accagcagaacagcacgatggccgtgagccacaccaggatgagcttgaccatggagctccgcgtcagggacttcaccatccccaccacagagaagctggccttggtccaccagcgcaacagca

SEQ ID NO: 3 - DNA, 23 bp, Artificial Sequence
  Synthetic CRISPR sequence
cattctccagcgctccgccacgg

SEQ ID NO: 4 - DNA, 127 bp, Artificial Sequence
  Synthetic CRISPR sequence
ggcagccctggtcatgtactggaagctcaaccccaagaagaagaagcaggtggccgtggcagagctgctggagaatgtcggccaggtcaacgagcacggtgcgaggattcaccctgggcaccagcct

SEQ ID NO: 5 - DNA, 17 bp, Artificial Sequence
  Synthetic primer
caatgccacgcttctgc

SEQ ID NO: 6 - DNA, 20 bp, Artificial Sequence
  Synthetic primer
ttctacacacccaagacccg

SEQ ID NO: 7 - DNA, 20 bp, Artificial Sequence
  Synthetic primer
tgacctcaacgatgcatttc

SEQ ID NO: 8 - DNA, 20 bp, Artificial Sequence
  Synthetic primer
ctgtcctggctcttctgctc

SEQ ID NO: 9 - DNA, 20 bp, Artificial Sequence
  Synthetic primer
atgcccggaactttttcttt

SEQ ID NO: 10 - DNA, 20 bp, Artificial Sequence
  Synthetic primer
catagagaacgtggcagcaa

SEQ ID NO: 11 - DNA, 19 bp, Artificial Sequence
  Synthetic primer
tgggttcagacagcagctc

SEQ ID NO: 12 - DNA, 20 bp, Artificial Sequence
  Synthetic primer
cccagactccgtcagtttct

SEQ ID NO: 13 - DNA, 20 bp, Artificial Sequence
  Synthetic primer
agctgccttgtaccagcatt

SEQ ID NO: 14 - DNA, 21 bp, Artificial Sequence
  Synthetic primer
tgctctctcttcacctgctct

SEQ ID NO: 15 - DNA, 18 bp, Artificial Sequence
  Synthetic primer
cgtccgcttgttctcctc

SEQ ID NO: 16 - DNA, 20 bp, Artificial Sequence
  Synthetic primer
cctttcccatggatgaagtc

SEQ ID NO: 17 - DNA, 19 bp, Artificial Sequence
  Synthetic primer
ggagcttgagtcctgaggg

SEQ ID NO: 18 - DNA, 20 bp, Artificial Sequence
  Synthetic primer
tctacgacagcagcgacaac

SEQ ID NO: 19 - DNA, 20 bp, Artificial Sequence
  Synthetic primer
ccgagtcctgcttcttcttg

SEQ ID NO: 20 - DNA, 20 bp, Artificial Sequence
  Synthetic primer
attcgttggggatgacagag

SEQ ID NO: 21 - DNA, 20 bp, Artificial Sequence
  Synthetic primer
tcacgaagtcgttcttgctg

SEQ ID NO: 22 - DNA, 20 bp, Artificial Sequence
  Synthetic primer
catgctttgttagggatggg

SEQ ID NO: 23 - DNA, 20 bp, Artificial Sequence
  Synthetic primer
ggccagaggctttgatacct

SEQ ID NO: 24 - DNA, 20 bp, Artificial Sequence
  Synthetic primer
gcaagaggcaaagagaatcg

SEQ ID NO: 25 - DNA, 20 bp, Artificial Sequence
  Synthetic primer
aaggggatttccatgtctcc

SEQ ID NO: 26 - DNA, 20 bp, Artificial Sequence
  Synthetic primer
cctggtgttagagacgcagc

SEQ ID NO: 27 - DNA, 20 bp, Artificial Sequence
  Synthetic primer
tggatcagtctggaaaagca

SEQ ID NO: 28 - DNA, 20 bp, Artificial Sequence
  Synthetic primer
agccaaaatcagagctggaa

SEQ ID NO: 29 - DNA, 21 bp, Artificial Sequence
  Synthetic primer
tgtcttttgtcaggggtcttt

SEQ ID NO: 30 - DNA, 20 bp, Artificial Sequence
  Synthetic primer
cacagtggtgcctaccaaga

SEQ ID NO: 31 - DNA, 21 bp, Artificial Sequence
  Synthetic primer
atccatggggagatgttctgg

SEQ ID NO: 32 - DNA, 21 bp, Artificial Sequence
  Synthetic primer
ctgagtccgaatcaggtgcag

SEQ ID NO: 33 - DNA, 20 bp, Artificial Sequence
  Synthetic primer
aggaagctcaaagccgaact

SEQ ID NO: 34 - DNA, 20 bp, Artificial Sequence
  Synthetic primer
acgcttcttctggaagacca

SEQ ID NO: 35 - DNA, 20 bp, Artificial Sequence
  Synthetic primer
gccataaggcatcattggac

SEQ ID NO: 36 - DNA, 20 bp, Artificial Sequence
  Synthetic primer
aacaacagcctgccacctta

SEQ ID NO: 37 - DNA, 20 bp, Artificial Sequence
  Synthetic primer
cgtcccacctagaatctgct

SEQ ID NO: 38 - DNA, 20 bp, Artificial Sequence
  Synthetic primer
ttgctcacaaaggtcacagg

SEQ ID NO: 39 - DNA, 18 bp, Artificial Sequence
  Synthetic primer
atgctggacgacagagcc

SEQ ID NO: 40 - DNA, 20 bp, Artificial Sequence
  Synthetic primer
ccttcttcaggtcctcctcc

SEQ ID NO: 41 - DNA, 20 bp, Artificial Sequence
  Synthetic primer
catagagaacgtggcagcaa

SEQ ID NO: 42 - DNA, 20 bp, Artificial Sequence
  Synthetic primer
atgcccggaactttttcttt

SEQ ID NO: 43 - DNA, 20 bp, Artificial Sequence
  Synthetic primer
gcatggtttccaggtcatct

SEQ ID NO: 44 - DNA, 20 bp, Artificial Sequence
  Synthetic primer
agtccctccaacaacagcaa

SEQ ID NO: 45 - DNA, 20 bp, Artificial Sequence
  Synthetic primer
tggtagctggtaacagcagg

SEQ ID NO: 46 - DNA, 20 bp, Artificial Sequence
  Synthetic primer
caggaactcagggagtgagc

SEQ ID NO: 47 - DNA, 21 bp, Artificial Sequence
  Synthetic primer
cctggtgattcaggtgggagt

SEQ ID NO: 48 - DNA, 20 bp, Artificial Sequence
  Synthetic primer
ggttgattgcctcgacacca

SEQ ID NO: 49 - DNA, 20 bp, Artificial Sequence
  Synthetic primer
tggaaacagaggaccagcac

SEQ ID NO: 50 - DNA, 20 bp, Artificial Sequence
  Synthetic primer
gttggagttggagcgaggtt

SEQ ID NO: 51 - DNA, 20 bp, Artificial Sequence
  Synthetic primer
ccaaggtgcaacttccttcg

SEQ ID NO: 52 - DNA, 20 bp, Artificial Sequence
  Synthetic primer
gatctcgttggggtcgaact

SEQ ID NO: 53 - DNA, 20 bp, Artificial Sequence
  Synthetic primer
tcccaaaagacaagccatgc

SEQ ID NO: 54 - DNA, 22 bp, Artificial Sequence
  Synthetic primer
aaccactgacatgacaaaagcg

SEQ ID NO: 55 - DNA, 20 bp, Artificial Sequence
  Synthetic primer
cgccctcctaatggtcaagg

SEQ ID NO: 56 - DNA, 20 bp, Artificial Sequence
  Synthetic primer
ttctgaggaccagtagggca

SEQ ID NO: 57 - DNA, 20 bp, Artificial Sequence
  Synthetic primer
atccactgcacgcaaactct

SEQ ID NO: 58 - DNA, 20 bp, Artificial Sequence
  Synthetic primer
ctgccttcactctcggacat

SEQ ID NO: 59 - DNA, 20 bp, Artificial Sequence
  Synthetic primer
aagcccctggtggagaagaa

SEQ ID NO: 60 - DNA, 19 bp, Artificial Sequence
  Synthetic primer
cttcggcgttctccagctt

SEQ ID NO: 61 - DNA, 20 bp, Artificial Sequence
  Synthetic primer
aagccgcccagttcaataca

SEQ ID NO: 62 - DNA, 22 bp, Artificial Sequence
  Synthetic primer
cctggaagagcacataaaccga

SEQ ID NO: 63 - DNA, 20 bp, Artificial Sequence
  Synthetic primer
acgctaagagcttcgtgctg

SEQ ID NO: 64 - DNA, 20 bp, Artificial Sequence
  Synthetic primer
tgaagcgagggttgaagtgc

SEQ ID NO: 65 - DNA, 25 bp, Artificial Sequence
  Synthetic primer
tttgtgttatgaatcacatgaaagc

SEQ ID NO: 66 - DNA, 20 bp, Artificial Sequence
  Synthetic primer
ggcttccctattgagctcgt

SEQ ID NO: 67 - DNA, 20 bp, Artificial Sequence
  Synthetic primer
ccgagttcacaggtctcacc

SEQ ID NO: 68 - DNA, 20 bp, Artificial Sequence
  Synthetic primer
ggtctcgcacttgttgagga

SEQ ID NO: 69 - DNA, 20 bp, Artificial Sequence
  Synthetic primer
gccatagagggcaaagtggt

SEQ ID NO: 70 - DNA, 20 bp, Artificial Sequence
  Synthetic primer
gttcccctagagacctccgt

SEQ ID NO: 71 - DNA, 22 bp, Artificial Sequence
  Synthetic primer
ttgattgatcctcaggcacgtc

SEQ ID NO: 72 - DNA, 20 bp, Artificial Sequence
  Synthetic primer
agtggcaagaaggcaggact

SEQ ID NO: 73 - DNA, 20 bp, Artificial Sequence
  Synthetic primer
agaagggggaagaaacgtcc

SEQ ID NO: 74 - DNA, 25 bp, Artificial Sequence
  Synthetic primer
ccctgttagccatctaatcaagtct

SEQ ID NO: 75 - DNA, 20 bp, Artificial Sequence
  Synthetic primer
gggtctctggtgctcttcac

SEQ ID NO: 76 - DNA, 20 bp, Artificial Sequence
  Synthetic primer
agaggacaccattccgacac

SEQ ID NO: 77 - DNA, 18 bp, Artificial Sequence
  Synthetic primer
ctccggcttcccctctcc

SEQ ID NO: 78 - DNA, 20 bp, Artificial Sequence
  Synthetic primer
gcgtacgaggacattttgcc

SEQ ID NO: 79 - DNA, 20 bp, Artificial Sequence
  Synthetic primer
gcgctccattaaatagccgt

SEQ ID NO: 80 - DNA, 20 bp, Artificial Sequence
  Synthetic primer
ctctcatggcgcagaggttt

SEQ ID NO: 81 - DNA, 20 bp, Artificial Sequence
  Synthetic primer
ctgacgacgtgaaggagcag

SEQ ID NO: 82 - DNA, 20 bp, Artificial Sequence
  Synthetic primer
caaacggtgaatccggctct

SEQ ID NO: 83 - DNA, 23 bp, Artificial Sequence
  Synthetic primer
gatttcacttccactccataacg

SEQ ID NO: 84 - DNA, 20 bp, Artificial Sequence
  Synthetic primer
cttctaggggatttagcggg

SEQ ID NO: 85 - DNA, 25 bp, Artificial Sequence
  Synthetic primer
gaattgcgtcatttaaagcctagtt

SEQ ID NO: 86 - DNA, 26 bp, Artificial Sequence
  Synthetic primer
gtttcatcctaccactcccaattaat

SEQ ID NO: 87 - DNA, 21 bp, Artificial Sequence
  Synthetic primer
tctcatggcctcttggttcaa

SEQ ID NO: 88 - DNA, 20 bp, Artificial Sequence
  Synthetic primer
tcgagagctgattgggaagc

SEQ ID NO: 89 - DNA, 20 bp, Artificial Sequence
  Synthetic primer
aaatcacatccgccccaact

SEQ ID NO: 90 - DNA, 20 bp, Artificial Sequence
  Synthetic primer
ggcggtgtctcaaaagggat

SEQ ID NO: 91 - DNA, 20 bp, Artificial Sequence
  Synthetic primer
aaaagtccgcacattcgagc

SEQ ID NO: 92 - DNA, 20 bp, Artificial Sequence
  Synthetic primer
cgctgctagttctcagtgct

SEQ ID NO: 93 - DNA, 24 bp, Artificial Sequence
  Synthetic primer
tggctcctttcacctttcttacac

SEQ ID NO: 94 - DNA, 20 bp, Artificial Sequence
  Synthetic primer
ctgcgtccagaggtcagttg

SEQ ID NO: 95 - DNA, 20 bp, Artificial Sequence
  Synthetic primer
cgtccagagcaacttacccc

SEQ ID NO: 96 - DNA, 21 bp, Artificial Sequence
  Synthetic primer
catgtttattgtggggccagg

SEQ ID NO: 97 - DNA, 22 bp, Artificial Sequence
  Synthetic primer
aggagttagccaagatgtgact

SEQ ID NO: 98 - DNA, 20 bp, Artificial Sequence
  Synthetic primer
agtcattggggtagaagcgg

SEQ ID NO: 99 - DNA, 20 bp, Artificial Sequence
  Synthetic primer
catgaaggagcacccggatt

SEQ ID NO: 100 - DNA, 20 bp, Artificial Sequence
  Synthetic primer
ttcatgtgcgcgtaactgtc

SEQ ID NO: 101 - DNA, 20 bp, Artificial Sequence
  Synthetic primer
tgcccacttgacttcaccaa

SEQ ID NO: 102 - DNA, 20 bp, Artificial Sequence
  Synthetic primer
gttcacagtccttcgctcct

SEQ ID NO: 103 - DNA, 20 bp, Artificial Sequence
  Synthetic primer
gctccttcttgccaaccaac

SEQ ID NO: 104 - DNA, 20 bp, Artificial Sequence
  Synthetic primer
gcccagagagaggtaggtgt

SEQ ID NO: 105 - DNA, 20 bp, Artificial Sequence
  Synthetic primer
tgttcactggtacgtgggtg

SEQ ID NO: 106 - DNA, 22 bp, Artificial Sequence
  Synthetic primer
gctgaaattctgggagcatgac